SEQUENCE LISTING (17 sequences)

SEQ ID NO: 1 - protein, 245 aa, Streptococcus suis
MTTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYITPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 2 - protein, 10 aa, Streptococcus suis
PPGTVAQSAP

SEQ ID NO: 3 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 4 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPNTSGEIVAVYKRGEHFDYDTVIEDVNGYVWVSYIGGSGKRNYVATGATKDGKRFGNAWGTFK

SEQ ID NO: 5 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPNTSGEIVAVYKRGEQFDYDTAIIDVNGYAWVSYIGGSGKRNYVATGATKDGKRFGNAWGTFK

SEQ ID NO: 6 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPDTSGEIVAVYKRGEQFDYDTAIIDVNGYAWVSYIGGSGKRNYVATGATKDGKRFGNAWGTFK

SEQ ID NO: 7 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPDTSGEIVAVYKRGESFDYDTVIIDVNGYVWVSYIGGSGKRNYVATGATKDGKRFGNAWGTFK

SEQ ID NO: 8 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSNRETGTMTVTVDALNVRRAPNTSGEIVAVYKEGESFDYDTVIIDVNGYVWVSYIGGSGKRNYVATGATKDGKRFGNAWGTFK

SEQ ID NO: 9 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPDTSGEIVAVYKRGEHFDYDTVIIDVNGYVWVSYIGGSGKRNYVATGATKDGKRFGNAWGTFK

SEQ ID NO: 10 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPDTSGEIVAVYKRGESFDYDTAIIDVNGYAWVSYIGGSGKRNYVATGATKDGKRFGNAWGTFK

SEQ ID NO: 11 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYITPPGTVAQSAPNLAGSRSNRETGTMTVTVDALNVRRAPDTSGEIVAVYKEGESFDYDTEIIDVNGYVWVSYIGGSGKRNYVATGATKDGKRFGNAWGTFK

SEQ ID NO: 12 - protein, 245 aa, Artificial Sequence
  Description of Artificial Sequence Synthetic polypeptide
MTTVNEALNNVRAQVGSGVSVGNGECYALASWYEEMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 13 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPTRNYYSAASSRQQVVHYITPPGTVAQSAPNLAGSRSKRETGTMTVTVDALNVRRAPNTSGEIVAVYKRGESFDYDTVIIDVNGYVWVSYIGGSGKRNYVATGATKDGKRFGNAWGTFK

SEQ ID NO: 14 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPNTSGEIVAVYKRGESFDYDTVIIDVNGYVWVSYIGGSGKRNYVATGATKDGKRFGNAWGTFK

SEQ ID NO: 15 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPNTSGEIVAVYKRGESFDYDTKIIDVNGYEWVSYIGGSGKRNYVATGATKDGKRFGNAWGTFK

SEQ ID NO: 16 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPDTSGEIVAVYKRGEQFDYDTKIIDVNGYEWVSYIGGSGKRNYVATGATKDGKRFGNAWGTFK

SEQ ID NO: 17 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPNTSGEIVAVYKRGESFDYDTVIEDVNGYVWGSYIGGSGKRNYVATGATKDGKRFGNAWGTFK

(19) United States
(12) Patent Application Publication
(10) Pub. No.: US 2021/0032294 A1
(43) Pub. Date: Feb. 4, 2021

(54) MODIFIED PlySs2 LYSINS AND USES THEREOF

(71) Applicant: CONTRAFECT CORPORATION, Yonkers, NY (US)

(72) Inventors: Raymond SCHUCH, Mountain Lakes, NJ (US); Chiara INDIANI, Metuchen, NJ (US); Michael WITTEKIND, NE Bainbridge Island, WA (US)

(21) Appl. No.: 16/975,321

(22) PCT Filed: Feb. 26, 2019
(86) PCT No.: PCT/US19/19638

§ 371 Date: Aug. 24, 2020

Related U.S. Application Data
(60) Provisional application No. 62/635,515, filed on Feb. 26, 2018.

(51) Int. Cl. C07K 14/005; C12N 9/36; A61P 31/04

(52) U.S. Cl. CPC C07K 14/005; C12N 2795/00022; A61P 31/04; C12N 9/2462

(57) ABSTRACT

Disclosed herein are modified lysin polypeptides thereof comprising at least one amino acid substitution as compared to a wild-type PlySs2 lysin polypeptide having an amino acid sequence of SEQ ID NO: 1, wherein the at least one amino acid substitution is in the CHAP domain and/or the SH3b domain, and wherein the modified lysin polypeptide or fragment thereof inhibits the growth, tin reduces the population, or kills at least one species of Gram-positive bacteria. Further disclosed herein are compositions comprising the modified lysin polypeptides, as well as vectors comprising a nucleic acid molecule that encodes the modified lysin polypeptide. Also disclosed herein are methods of inhibiting the growth, reducing the population, or killing at least one species of Gram-positive bacteria, methods of treating a bacterial infection, and methods of augmenting the efficacy of an antibiotic or reducing the development of antibiotic resistance.

### CROSS-REFERENCE TO RELATED APPLICATIONS

This application claims the benefit of, and relies on the filing date of, U.S. provisional patent application No. 62/635,515, filed 26 Feb. 2018, the entire disclosure of which is incorporated herein by reference.

### SEQUENCE LISTING

The instant application contains a Sequence Listing which has been submitted electronically in ASCII format and is hereby incorporated by reference in its entirety. Said ASCII copy, created on Feb. 22, 2019, is named 0341_0004-PCT_SL.txt and is 36,153 bytes in size.

### FIELD OF THE DISCLOSURE

The present disclosure relates generally to antibacterial agents and more specifically to modified, non-naturally occurring lysin polypeptides, notably, modified PlySs2 lytic enzymes, and the use of these peptides in killing Gram-positive bacteria and combatting bacterial infection and contamination.

### BACKGROUND OF THE INVENTION

Antibiotic resistance is on the increase worldwide, influenced, inter alia, by (a) increased and prolonged use of antibiotics administered to treat a variety of illnesses and other conditions; (b) poor patient compliance; and (c) a paucity of new antimicrobial agents that can be deployed against pathogens that have developed resistance to existing antibiotics.

Bacteriophage endolysins (lysins) represent a promising alternative or complementary approach to combating bacterial infections and to overcoming bacterial resistance. Lysins are peptidoglycan hydrolases that can be produced naturally by bacteriophages. When contacting the bacteria from the outside, recombinantly-produced lysin polypeptides directly lyse and kill the bacteria [1], [2]. Lysins may also overcome antibiotic resistance by facilitating access of the antibiotic agents to pathogens. Several studies have recently demonstrated the strong potential of these enzymes in human and veterinary medicine to control pathogens on mucosal surfaces, in organ-confined infections, and in systemic infections.

Gram-positive bacteria are surrounded by a cell wall containing polypeptides and polysaccharides. The gram-positive cell wall appears as a broad, dense wall that may be about 20-80 nm thick and contains numerous interconnecting layers of peptidoglycan. Between 60% and 90% of the gram-positive cell wall is peptidoglycan, providing cell shape, a rigid structure, and resistance to osmotic shock. The cell wall does not exclude the Gram stain crystal violet, allowing cells to be stained purple, and therefore classified as “Gram-positive.”

Bacteriophage lytic enzymes have been established as useful in the specific treatment of various types of infection in subjects through various routes of administration. See e.g., U.S. Pat. Nos. 5,985,271; 6,017,528; 6,056,955; 6,248,324; 6,254,866; and 6,264,945. U.S. Pat. No. 9,034,322 to Fischetti et al., which is hereby incorporated by reference in its entirety, is directed to bacteriophage lysins derived from Streptococcus suis bacteria, including the lysin PlySs2. These lysin polypeptides demonstrate broad killing activity against multiple bacteria, including Gram-positive bacteria such as Staphylococcus, Streptococcus Group B, Enterococcus, and Listeria bacterial strains.

The PlySs2 lysin is capable of killing Staphylococcus aureus bacteria in animal models, synergizing with antibiotics, and overcoming (or preventing) antibiotic resistance. PlySs2 was shown to be effective against antibiotic-resistant Staphylococcus aureus, such as methicillin-resistant Staphylococcus aureus (MRSA) and vancomycin-resistant Staphylococcus aureus (VRSA).

The elicitation of an inflammatory immune response by a therapeutic protein is undesirable [6], [8]. Protein immunogenicity may be of concern for exogenous proteins and peptides, whether they are derived from synthetic or biological (such as recombinant) sources. In some instances, therapeutic proteins have caused severe adverse events (for example, anemia, thrombocytopenia, anaphylaxis, and innate immune responses) that can even be fatal in certain instances. Well-known examples include thrombocytopenia in patients treated with thrombopoietin and pure red cell aplasia in chronic renal disease patients treated with EPREX®, an approved erythropoietin (EPO) product, following induction of neutralizing antibodies that cross-reacted with the functionally non-redundant endogenous EPO. Since immunogenicity has been reported with various products, including monoclonal antibodies, thrombokinase, and PULMOZYME® (dornase alfa), immunogenicity continues to receive significant attention from regulatory bodies, industry, and clinicians. Such immune responses may be potentially exacerbated in a patient who is already experiencing inflammatory response because of the underlying bacterial infection.

As lysins, such as PlySs2, are proteins, they have the potential of eliciting an immune response when administered to a host. In addition to causing severe adverse effects, as discussed above, such immune responses can also decrease lytic activity of a lysin. Indeed, some immune responses have been observed in animals with other types of lysins, such as Cpl-1, which caused a slowdown in the lytic activity but did not substantially inhibit it. Other researchers, however, observed that administration of Cpl-1 was accompanied by a substantial increase in inflammatory cytokine secretion [9].

In silico, computationally-guided tools have been developed to facilitate the identification of epitopic regions of proteins and to design variants that are less predisposed to being immunogenic [11]-[18]. Although such techniques may be helpful, they have some limitations. These limitations may include, for example: antigen processing, which can eliminate some putative T-cell epitopes; inability to predict T-cell receptor affinity using in silico methods due to the pleiomorphism and three-dimensional complexity of the T-cell receptor; inability to predict T-cell epitope phenotypes; limitations in the applicability of statistical techniques devised with patient populations in mind to an individual patient and influence of post-translational factors; and inherent limitations of in silico techniques and the assumptions underlying them [8]. Accordingly, there remains trial and error and uncertainty in efforts to reduce the immunogenicity of peptides.

Thus, the discovery of modified lysins, such as modified PlySs2 lysins, that retain desired antibacterial activity, yet have reduced immunogenicity, would be beneficial.

### SUMMARY OF THE DISCLOSURE

This application discloses modified lysin polypeptides having at least one amino acid substitution relative to a counterpart wild-type PlySs2 lysin, while preserving antibacterial activity and effectiveness. Typically, the modified lysin polypeptides also have reduced immunogenicity as compared to the counterpart wild-type PlySs2 lysin. The wild-type PlySs2 lysin has a cysteine, histidine-dependent amidohydrolase/peptidase (CHAP) endopeptidase domain, which is the enzymatically active domain (EAD) of the PlySs2 polypeptide, and a C-terminal SH3b_5 (SH3b) cell wall-binding domain (CBD). In certain aspects, the modified lysin polypeptides comprise at least one amino acid substitutions in the CHAP and/or one or more amino acid substitutions in the SH3b.

In one aspect, the present disclosure is directed to a modified lysin polypeptide, comprising at least one amino acid substitution as compared to a wild-type PlySs2 lysin polypeptide, wherein the wild-type PlySs2 lysin polypeptide has an amino acid sequence of SEQ ID NO: 1, a cysteine, histidine-dependent amidohydrolase/peptidase (CHAP) domain, and a cell wall binding (SH3b) domain, and wherein the at least one amino acid substitution is in the CHAP domain and/or the SH3b domain, wherein the modified lysin polypeptide inhibits the growth, reduces the population, or kills at least one species of Gram-positive bacteria. Typically, the modified lysin polypeptide has reduced immunogenicity as compared to the wild-type PlySs2 (SEQ ID NO: 1). In certain embodiments, the at least one amino acid substitution is in the CHAP domain. In certain embodiments, the at least one amino acid substitution is in the SH3b domain. In certain embodiments, the at least one amino acid substitution is in the CHAP domain and the SH3b domain.

In certain embodiments, the at least one substitution is in the CHAP domain in at least one position selected from amino acid residue 35, 92, 104, 128, and 137 of SEQ ID NO: 1. In certain embodiments, the at least one substitution is in the SH3b domain in at least one position selected from amino acid residue 164, 184, 195, 198, 204, 206, 212, and 214 of SEQ ID NO: 1. In certain embodiments, modified lysin polypeptide has at least one substitution in the CHAP domain in at least one position selected from amino acid 35, 92, 104, 128, and 137 of SEQ ID NO: 1 and at least one substitution in the SH3b domain in at least one position selected from amino acid 164, 184, 195, 198, 204, 206, 212, and 214 of SEQ ID NO: 1.

In some embodiments, the at least one amino acid substitution in the CHAP domain is selected from the group consisting of R35E, L92W, V104S, V128T and Y137S. In certain embodiments, the at least one amino acid substitution in the SH3b domain is selected from the group consisting of Y164N, Y164K, N184D, R195E, S198H, S198Q, V204K, V204A, I206E, V212A, V212E, and V214G.

In certain embodiments, the modified lysin polypeptide has at least one amino acid substitution in the CHAP domain selected from the group consisting of R35E, L92W, V104S, V128T and Y137S and at least one amino acid substitution in the SH3b domain selected from the group consisting of Y164N, Y164K, N184D, R195E, S198H, S198Q, V204K, V204A, I206E, V212A, V212E, and V214G.

In yet other embodiments, the modified lysin polypeptide has at least two amino acid substitutions in the CHAP domain; in still other embodiments, the modified lysin polypeptide has at least two amino acid substitutions in the SH3b domain; in other embodiments, the modified lysin polypeptide has at least three amino acid substitutions in the SH3b domain. In yet other embodiments, the modified lysin polypeptide has 5, 6, 7, or 8 amino acid substitutions distributed between the CHAP and SH3b domains, and in certain embodiments, the amino acid sequence of SEQ ID NO: 1 is modified by 3-9 of the amino acid substitutions selected from the group consisting of: R35E, L92W, V104S, V128T, Y137S, Y164N, Y164K, N184D, R195E, S198H, S198Q, V204K, V204A, 1206E, V212E, V212A, and V214G.

In certain embodiments, the modified lysin polypeptide comprises the following amino acid substitutions relative to the amino acid sequence of SEQ ID NO: 1: (i) L92W, V104S, V128T, and Y137S (pp55); (ii) Y164N, N184D, R195E, V204K, and V212E (pp388); (iii) L92W, V104S, V128T, Y137S, S198H, and I206E (pp61); (iv) L92W, V104S, V128T, Y137S, S198Q, V204A, and V212A (pp65); (v) L92W, V104S, V128T, Y137S, Y164K, N184D, and S198Q (pp296); (vi) V128T, Y137S, and Y164K (pp616); (vii) R35E, L92W, V104S, V128T, and Y137S (pp400); (viii) L92W, V104S, V128T, Y137S, Y164K, V204K, and V212E (pp628); (ix) L92W, V104S, V128T, Y137S, Y164K, N184D, S198Q, V204K, and V212E (pp632); (x) L92W, V104S, V128T, Y137S, Y164N, and N184D (pp324); (xi) L92W, V104S, V128T, Y137S, Y164N, and R195E (pp325); (xii) L92W, V104S, V128T, Y137S, N184D, V204A, and V212A (pp341); (xiii) L92W, V104S, V128T, Y137S, and Y164K (pp619); (xiv) L92W, V104S, V128T, Y137S, Y164K, I206E, and V214G (pp642); and (xv) L92W, V104S, V128T, Y137S, N184D, and S198H (pp338). In certain embodiments, the modified lysin polypeptide has an amino acid sequence selected from one of SEQ ID NOs. 3-17.

In certain embodiments the modified lysin polypeptide comprises the following amino acid substitutions relative to the amino acid sequence of SEQ ID NO: 1: L92W, V104S, V128T, and Y137S. In certain embodiments the modified lysin polypeptide comprises the following amino acid substitutions relative to the amino acid sequence of SEQ ID NO: 1: L92W, V104S, V128T, Y137S, Y164K, N184D, and S198Q (pp296).

Also disclosed are active fragments of the modified lysin polypeptides disclosed herein, where the active fragments include one or more of the amino acid substitutions in the CHAP domain and/or the SH3b domain.

Also disclosed are chimeric lysins comprising a modified PlySs2 CHAP domain, as disclosed herein, and the binding domain of another lysin or the catalytic domain of another lysin and a modified PlySs2 SH3b domain, as disclosed herein.

In some embodiments, the modified lysin polypeptide has a minimum inhibitory concentration (MIC) no greater than about 2, or a MIC that is about 3 to about 5 times, such as no greater than about 3 times, no greater than about 4 times, or no greater than about 5 times, than that of the wild-type PlySs2 lysin (SEQ ID NO: 1) against one or more of Staphylococcus aureus, Listeria monocytogenes, a coagulase negative staphylococcus (including at least 40 recognized species from, but not limited to, the Staphylococcus epidermidis group, the Staphylococcus saprophyticus group, the Staphylococcus simulans group, the Staphylococcus intermedius group, the Staphylococcus sciuri group, the Staphylococcus hyicus group, and any isolates referred to as from the “unspecified species group”), Streptococcus suis, Streptococcus pyogenes, Streptococcus agalactiae, Streptococcus dysgalactiae, Streptococcus pneumoniae, any additional species included in the viridans streptococci group (including, but not limited to, all species and strains included in the Streptococcus anginosis group, Streptococcus mitis group, Streptococcus sanguinis group, Streptococcus bovis (now gallolyticus) group, Streptococcus salivarius group, and Streptococcus mutans group), Enterococcus faecalis, and Enterococcus faecium. In certain embodiments, the MIC is no greater than about 2, no greater than about 3, no greater than about 4, or no greater than about 5 times that of the wild-type PlySs2 lysin (SEQ ID NO: 1) against one or more of Staphylococcus aureus, Streptococcus pyogenes, Listeria monocytogenes, and Streptococcus agalactiae.

In some embodiments, the modified lysin polypeptides disclosed herein reduce immunogenicity and/or reduce inflammatory response-related toxicity compared to wild-type PlySs2 lysin (SEQ ID NO: 1). In certain embodiments of the disclosure, inhibiting the growth, reducing the population, or killing at least one species of Gram-positive bacteria is assessed in vitro as the MIC and/or minimum biofilm eradication concentration (MBEC).

Another aspect is directed to compositions comprising an acceptable carrier and a modified lysin polypeptide as disclosed herein. In certain embodiments, the composition is a pharmaceutical composition and the carrier is a pharmaceutically-acceptable carrier.

In particular embodiments of the compositions disclosed herein, the amount of the modified lysin polypeptide is effective to inhibit the growth, reduce the population, or kill one or more species of the Gram-positive bacteria, such as a methicillin-resistant Staphylococcus aureus or a vancomycin-resistant Staphylococcus aureus. In certain embodiments, the composition is a solution, a suspension, an emulsion, an inhalable powder, an aerosol, or a spray. In certain embodiments, the composition further comprises one or more antibiotics suitable for treatment of a Gram-positive bacterial infection.

In yet another aspect, a nucleic acid molecule is provided that encodes a modified lysin polypeptide as disclosed herein.

Another aspect is directed to a vector comprising a nucleic acid molecule that encodes a modified lysin polypeptide as disclosed herein. In some embodiments, the vector is a plasmid. In some embodiments, the nucleic acid molecule is operatively linked to a heterologous promoter.

In another aspect, a method is provided for inhibiting the growth, reducing the population, or killing at least one species of Gram-positive bacteria, the method comprising contacting the least one species of Gram-positive bacteria with a composition comprising an antibacterial effective amount of a modified lysin polypeptide as disclosed herein.

In still another aspect, a method is provided for preventing or treating a bacterial infection caused by at least one species of Gram-positive bacteria comprising co-administering to a subject diagnosed with, at risk for, or exhibiting symptoms of the bacterial infection (1) a first amount of a modified lysin polypeptide as disclosed herein; and (2) a second amount of an antibiotic suitable for the treatment of a Gram-positive bacterial infection.

In some method embodiments, the antibiotic is one or more of methicillin, vancomycin, daptomycin, mupirocin, and lysostaphin. In certain embodiments, the antibiotic is one or more of methicillin, vancomycin, and daptomycin.

In some embodiments, the amount of the modified lysin polypeptide used in the foregoing methods may be below that which would result in a concentration equal to the MIC of the modified lysin polypeptide when used in the absence of antibiotic (i.e., a “sub-MIC lysin amount”); alternatively or additionally, the amount of antibiotic used in the foregoing methods may be below that which corresponds, i.e., which would result in, a concentration equal to the MIC for the antibiotic when used in the absence of the modified lysin polypeptide (i.e., a “sub-MIC antibiotic amount”).

In still another aspect, a method is provided for augmenting the efficacy of an antibiotic suitable for the treatment of a Gram-positive bacterial infection, comprising co-administering the antibiotic in combination with a modified lysin polypeptide as disclosed herein, wherein co-administration is more effective in inhibiting the growth, or reducing the population, or killing the Gram-positive bacteria than administration of either the antibiotic or the modified lysin polypeptide individually. In certain embodiments, the antibiotic is selected from the group consisting of methicillin, vancomycin, daptomycin, mupirocin, and lysostaphin.

In another aspect, a combination is provided, wherein the combination comprises a modified lysin polypeptide and an antibiotic. In certain embodiments, the minimum amount of the modified lysin polypeptide, the antibiotic, or both that is effective in the combination is below the respective MIC amount of the modified lysin polypeptide and/or the antibiotic. In some embodiments, the modified lysin polypeptide and the antibiotic are provided in the same composition, and in certain embodiments, the modified lysin polypeptide and the antibiotic are provided in different compositions.

In some embodiments, a method is provided for preventing, disrupting, dispersing, or treating a biofilm containing Staphylococcus or Streptococcus bacteria on a surface, comprising delivering to the surface an effective amount of a modified lysin polypeptide as disclosed herein, alone or in combination with an antibiotic, wherein the biofilm is effectively prevented, disrupted, dispersed, or treated. In certain embodiments, the surface is a surface of a medical device. In still other embodiments, the medical device is for use within or in contact with the human body, including without limitation blood or other bodily fluids. Nonlimiting examples of such devices include an inhaler, intubation equipment, valve, catheter, colostomy device, or other prosthetic device.

In certain embodiments, the activity of the modified lysin polypeptide, such as the ability of the peptide to inhibit growth, reduce the population, or kill at least one species of Gram-positive bacteria, may be assessed in vitro, for example as the MIC and/or minimum biofilm eradication concentration (MBEC). In certain embodiments, the activity of the modified lysin polypeptide may be assessed in vivo, for example by a mouse neutropenic thigh infection (MNTI) model. In further embodiments, the synergistic activity of the modified lysin polypeptides disclosed herein with antibiotics suitable for treating Gram-positive infections, for example, one or more of methicillin, vancomycin, daptomycin, mupirocin, and lysostaphin, may be assessed using a checkerboard assay.

In some certain embodiments, the modified lysin polypeptide may display an antibiotic resistance profile comparable to that of wild-type PlySs2 lysin. Accordingly, a method is provided for inhibiting the development of resistance to an antibiotic suitable for treatment of Staphylococcus or Streptococcus infections comprising co-administering an antibiotic with an amount of a modified lysin polypeptide as disclosed herein, effective to avert, reduce, or delay the development of resistance to the antibiotic.

In further embodiments, the antibiotic resistance profile is as assessed by serial passage resistance alone or by serial passage in combination with an antibiotic, such as, for example, methicillin, vancomycin, daptomycin, mupirocin, and lysostaphin.

### BRIEF DESCRIPTION OF THE FIGURES

FIG. 1 is a depiction of SDS-PAGE gels of fractions eluted from a size exclusion column during a final step of the purification process of modified lysin polypeptides pp53, pp55, pp61, pp65, and pp296, as well as a control, wild-type PlySs2 lysin protein pp1149 (purified to GMP grade), as described in Example 3. The fractions included in the drawn circles were pooled together in this final purification step.

FIG. 2 is a dose response plot of the bactericidal efficacy of wild-type PlySs2 lysin (designated as CF-301) and the control, wild-type PlySs2 polypeptide pp1149, as well as modified lysin polypeptides pp55, pp61, pp65, and pp296, in a mouse neutropenic thigh infection in vivo model, as described in Example 7. The bacterial burden (colony forming units/g of treated thigh) is plotted against the lysin dose administered (mg/kg).

FIGS. 3A-3C are a series of serial passage resistance assay results depicting the fold change in MIC over time for each indicated agent over the 21 or 26 days of passage, as described in Example 6. In FIG. 3A, the fold change in MIC (measured as increase in MIC) is shown for three independent lineages treated with pp296 (designated as “pp296-1”, “pp296-2”, and “pp296-3”) and a single lineage treated with either wild-type PlySs2 lysin (designated as CF-301) or lysostaphin (LSP). In FIG. 3B, the fold increase in the MIC of daptomycin (DAP) is shown for three independent lineages treated with DAP in combination with a fixed sub-MIC amounts ( 1/16×MIC) of pp296 (designated as “DAP+pp296-1,” “DAP+pp296-2,” and “DAP+pp-296-3”) and with DAP alone. In FIG. 3C, the fold increase in the MIC of vancomycin (VAN) is shown for three independent lineages treated with VAN in combination with a fixed sub-MIC amount (⅛×MIC) of pp296 (designated as “VAN+pp296-1,” “VAN+pp296-2,” and “VAN+pp-296-3”) and with VAN alone.

### DETAILED DESCRIPTION

### Definitions

As used herein, the following terms and cognates thereof shall have the following meanings unless the context clearly indicates otherwise:

“Carrier,” refers to a solvent, additive, excipient, dispersion medium, solubilizing agent, coating, preservative, isotonic and absorption delaying agent, surfactant, propellant, diluent, vehicle and the like with which an active compound is administered. Such carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil, and the like.

“Pharmaceutically acceptable carrier” refers to any and all solvents, additives, excipients, dispersion media, solubilizing agents, coatings, preservatives, isotonic and absorption delaying agents, surfactants, propellants, diluents, vehicles and the like that are physiologically compatible. The carrier(s) must be “acceptable” in the sense of not being deleterious to the subject to be treated in amounts typically used in medicaments. Pharmaceutically acceptable carriers are compatible with the other ingredients of the composition without rendering the composition unsuitable for its intended purpose. Furthermore, pharmaceutically acceptable carriers are suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers or excipients include any of the standard pharmaceutical carriers such as phosphate buffered saline solutions, water, and emulsions such as oil/water emulsions and microemulsions. Suitable pharmaceutical carriers are described, for example, in Remington's Pharmaceutical Sciences by E. W. Martin, 18th Edition.

“Bactericidal” refers to the property of causing the death of bacteria or capable of killing bacteria to an extent of at least a 3-log 10 (99.9%) or better reduction among an initial population of bacteria over an 18-24 hour period.

“Bacteriostatic” refer to the property of inhibiting bacterial growth, including inhibiting growing bacterial cells, thus causing a 2-log 10 (99%) or better and up to just under a 3-log reduction among an initial population of bacteria over an 18-24 hour period.

“Antibacterial” refers to both bacteriostatic and bactericidal agents.

“Antibiotic” refers to a compound having properties that have a negative effect on bacteria, such as lethality or reduction of growth. An antibiotic can have a negative effect on Gram-positive bacteria, Gram-negative bacteria, or both. By way of example, an antibiotic can affect cell wall peptidoglycan biosynthesis, cell membrane integrity, or DNA or protein synthesis in bacteria. Nonlimiting examples of antibiotics active against Gram-positive bacteria include methicillin, vancomycin, daptomycin, mupirocin, lysostaphin, penicillins, cloxacillin, erythromycin, carbapenems, cephalosporins, glycopeptides, lincosamides, azithromycin, clarithromycin, roxithromycin, telithromycin, spiramycin, and fidaxomicin.

“Drug resistant” generally refers to a bacterium that is resistant to the antibacterial activity of a drug. When used in certain ways, drug resistance may specifically refer to antibiotic resistance. In some cases, a bacterium that is generally susceptible to a particular antibiotic can develop resistance to the antibiotic, thereby becoming a drug resistant microbe or strain. A “multi-drug resistant” (“MDR”) pathogen is one that has developed resistance to at least two classes of antimicrobial drugs, each used as monotherapy. For example, certain strains of S. aureus have been found to be resistant to several antibiotics including methicillin and/or vancomycin (Antibiotic Resistant Threats in the United States, 2013, U.S. Department of Health and Services, Centers for Disease Control and Prevention). One skilled in the art can readily determine if a bacterium is drug resistant using routine laboratory techniques that determine the susceptibility or resistance of a bacterium to a drug or antibiotic.

“Effective amount” refers to an amount which, when applied or administered in an appropriate frequency or dosing regimen, is sufficient to prevent, reduce, inhibit, or eliminate bacterial growth or bacterial burden or to prevent, reduce, or ameliorate the onset, severity, duration, or progression of the disorder being treated (for example, bacterial pathogen growth or infection), prevent the advancement of the disorder being treated, cause the regression of the disorder being treated, or enhance or improve the prophylactic or therapeutic effect(s) of another therapy, such as antibiotic or bacteriostatic therapy.

“Co-administer” is intended to embrace separate administration of two agents, such as a lysin peptide and an antibiotic or any other antibacterial agent in a sequential manner as well as administration of these agents in a substantially simultaneous manner, such as in a single mixture/composition or in doses given separately, but nonetheless administered substantially simultaneously to the subject, for example at different times in the same day or 24-hour period. Such co-administration of lysin peptides with one or more additional antibacterial agents can be provided as a continuous treatment lasting up to days, weeks, or months. Additionally, depending on the use, the co-administration need not be continuous or coextensive. For example, if the use were as a topical antibacterial agent to treat, e.g., a bacterial ulcer or an infected diabetic ulcer, the lysin polypeptide could be administered only initially within 24 hours of the first antibiotic use, and then the antibiotic use may continue without further administration of the lysin polypeptide.

“Subject” refers to a mammal, a plant, a lower animal, a single cell organism, or a cell culture. For example, the term “subject” is intended to include organisms, e.g., prokaryotes and eukaryotes, which are susceptible to or afflicted with bacterial infections, for example Gram-positive or Gram-negative bacterial infections. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or susceptible to infection by Gram-positive bacteria, whether such infection be systemic, topical or otherwise concentrated or confined to a particular organ or tissue.

“Polypeptide” is used interchangeably with the term “protein,” “peptide,” and refers to a polymer made from amino acid residues. In certain embodiments, the polypeptide has at least about 30 amino acid residues. The term may include not only polypeptides in isolated form, but also active fragments and derivatives thereof. The term “polypeptide” also encompasses fusion proteins or fusion polypeptides comprising a modified lysin polypeptide as described herein and maintaining the lysin function. Depending on context, a polypeptide can be a naturally-occurring polypeptide or a recombinant, engineered, or synthetically-produced polypeptide. A particular lysin polypeptide can be, for example, derived or removed from a native protein by enzymatic or chemical cleavage, or can be prepared using conventional peptide synthesis techniques (e.g., solid phase synthesis) or molecular biology techniques (such as those disclosed in Sambrook, J. et al., Molecular Cloning: A Laboratory Manual, Cold Spring Harbor Press, Cold Spring Harbor, N.Y. (1989)) or can be strategically truncated or segmented yielding active fragments, maintaining lytic activity against the same or at least one common target bacterium.

“Fusion polypeptide” refers to an expression product resulting from the fusion of two or more nucleic acid segments, resulting in a fused expression product typically having two or more domains or segments with different properties or functionality. In certain embodiments, the term “fusion polypeptide” also refers to a polypeptide or peptide comprising two or more heterologous polypeptides or peptides covalently linked, either directly or via an amino acid or peptide linker. The polypeptides forming the fusion polypeptide are typically linked C-terminus to N-terminus, although they can also be linked C-terminus to C-terminus, N-terminus to N-terminus, or N-terminus to C-terminus. The term “fusion polypeptide” can be used interchangeably with the term “fusion protein.” Thus, the open-ended expression “a polypeptide comprising” a certain structure includes larger molecules than the recited structure such as fusion polypeptides or constructs. The constructs referred to herein can be made as fusion polypeptides or as conjugates (by linking two or more moieties).

“Heterologous” refers to nucleotide, peptide, or polypeptide sequences that are not naturally contiguous. For example, in the context of the present disclosure, the term “heterologous” can be used to describe a combination or fusion of two or more peptides and/or polypeptides wherein the fusion peptide or polypeptide is not normally found in nature, such as for example a modified lysin polypeptide and a cationic and/or a polycationic peptide, an amphipathic peptide, a sushi peptide (Ding et al. Cell Mol Life Sci., 65(7-8):1202-19 (2008)), a defensin peptide (Ganz, T. Nature Reviews Immunology 3, 710-720 (2003)), a hydrophobic peptide, and/or an antimicrobial peptide which may have enhanced lytic activity. Included in this definition are two or more lysin polypeptides or active fragments thereof. These can be used to make a fusion polypeptide with lytic activity.

“Active fragment” refers to a portion of a polypeptide that retains one or more functions or biological activities of the isolated polypeptide from which the fragment was taken. As used herein, an active fragment of a modified lysin polypeptides inhibits the growth, or reduces the population, or kills at least one Gram-positive bacterial species, such as S. aureus.

“Amphipathic peptide” refers to a peptide having both hydrophilic and hydrophobic functional groups. In certain embodiments, secondary structure places hydrophobic and hydrophilic amino acid residues at opposite sides (e.g., inner side vs outer side when the peptide is in a solvent, such as water) of an amphipathic peptide. These peptides may in certain embodiments adopt a helical secondary structure, such as an alpha-helical secondary structure.

“Cationic peptide” refers to a peptide having a high percentage of positively charged amino acid residues. In certain embodiments, a cationic peptide has a pKa-value of 8.0 or greater. The term “cationic peptide” in the context of the present disclosure also encompasses polycationic peptides which are synthetically produced peptides composed of mostly positively charged amino acid residues, such as lysine and/or arginine residues. The amino acid residues that are not positively charged can be neutrally charged amino acid residues, negatively charged amino acid residues, and/or hydrophobic amino acid residues.

“Hydrophobic group” refers to a chemical group such as an amino acid side chain which has low or no affinity for water molecules but higher affinity for oil molecules. Hydrophobic substances tend to have low or no solubility in water or aqueous phases and are typically apolar but tend to have higher solubility in oil phases. Examples of hydrophobic amino acids include glycine (Gly), alanine (Ala), valine (Val), Leucine (Leu), isoleucine (Ile), proline (Pro), phenylalanine (Phe), methionine (Met), and tryptophan (Trp).

“Augmenting” as used herein refers to a degree of activity of an agent, such as antimicrobial activity, that is higher than it would be otherwise. “Augmenting” encompasses additive as well as synergistic (superadditive) effects.

“Synergistic” or “superadditive” refers to a beneficial effect brought about by two substances in combination that exceeds the sum of the effects of the two agents working independently. In certain embodiments the synergistic or superadditive effect significantly, i.e., statistically significantly, exceeds the sum of the effects of the two agents working independently. One or both active ingredients may be employed at a subthreshold level, i.e., a level at which if the active substance is employed individually produces no or a very limited effect. The effect can be measured by assays such as a checkerboard assay, described here.

“Treatment” refers to any process, action, application, therapy, or the like, wherein a subject, including a human being, is subjected to medical aid with the object of curing a disorder, eradicating a pathogen, or improving the subject's condition, directly or indirectly. Treatment also refers to reducing incidence, alleviating symptoms, eliminating recurrence, preventing recurrence, preventing incidence, reducing the risk of incidence, improving symptoms, improving prognosis, or combinations thereof. “Treatment” may further encompass reducing the population, growth rate, or virulence of the bacteria in the subject and thereby controlling or reducing a bacterial infection in a subject or bacterial contamination of an organ, tissue, or environment. Thus “treatment” that reduces incidence is effective to inhibit growth of at least one Gram-positive bacterium in a particular milieu, whether it be a subject or an environment. On the other hand, “treatment” of an already established infection refers to reducing the population, killing, inhibiting the growth, and/or eradicating the Gram-positive bacteria responsible for an infection or contamination.

The term “preventing” and includes the prevention of the incidence, recurrence, spread, onset, or establishment of a disorder such as a bacterial infection. It is not intended that the present disclosure be limited to complete prevention or to prevention of establishment of an infection. In some embodiments, the onset is delayed, or the severity of a subsequently contracted disease or the chance of contracting it is reduced, and such constitute examples of prevention. With specific reference to biofilm prevention, the term includes prevention of the formation of biofilm, for example by interfering with the adherence of bacteria on a surface of interest, such as the surface of a medical device (e.g., inhaler, catheter, intubation, valve, or other prosthesis).

“Contracted disease” refers to a disease manifesting with clinical or subclinical symptoms, such as the detection of fever, sepsis, or bacteremia, as well as disease that may be detected by growth of a bacterial pathogen (e.g., in culture) when symptoms associated with such pathology are not yet manifest. With respect to medical devices, in particular, a contracted disease shall include a biofilm containing bacteria, such as Staphylococcus or Streptococcus bacteria, and forming when such a device is in use.

[1] The term “derivative” in the context of a peptide or polypeptide (which as stated herein includes an active fragment) is intended to encompass, for example, a polypeptide modified to contain one or more chemical moieties other than an amino acid that do not substantially adversely impact or destroy the polypeptides's activity, such as lytic activity. The chemical moiety can be linked covalently to the peptide, e.g., via an amino terminal amino acid residue, a carboxy terminal amino acid residue, or at an internal amino acid residue. Such modifications may be natural or non-natural. In certain embodiments, a non-natural modification may include the addition of a protective or capping group on a reactive moiety, addition of a detectable label, such as antibody and/or fluorescent label, addition or modification of glycosylation, or addition of a bulking group such as PEG (pegylation) and other changes known to those skilled in the art. In certain embodiments, the non-natural modification may be a capping modification, such as N-terminal acetylations and C-terminal amidations. Exemplary protective groups that may be added to lysin polypeptides include, but are not limited to, t-Boc and Fmoc. Commonly used fluorescent label proteins such as, but not limited to, green fluorescent protein (GFP), red fluorescent protein (RFP), cyan fluorescent protein (CFP), yellow fluorescent protein (YFP), and mCherry, are compact proteins that can be bound covalently or noncovalently to a lysin polypeptide or fused to a lysin polypeptide without interfering with normal functions of cellular proteins. In certain embodiments, a polynucleotide encoding a fluorescent protein is inserted upstream or downstream of the lysin polynucleotide sequence. This will produce a fusion protein (e.g., Lysin Polypeptide::GFP) that does not interfere with cellular function or function of a lysin polypeptide to which it is attached. Polyethylene glycol (PEG) conjugation to proteins has been used as a method for extending the circulating half-life of many pharmaceutical proteins. Thus, in the context of lysin polypeptide derivatives, the term “derivative” encompasses lysin polypeptides chemically modified by covalent attachment of one or more PEG molecules. It is anticipated that pegylated lysin polypeptides will exhibit prolonged circulation half-life compared to the unpegylated lysin polypeptides, while retaining biological and therapeutic activity. Another example is the use of “artilysins”, whereby a short polycationic and amphipathic alpha helices are appended to the N- or C-termini of a lysin polypeptide to improve in vitro antibacterial activity, such as a streptococcal lysin to improve in vitro anti-streptococcal activity.

“Percent amino acid sequence identity” refers to the percentage of amino acid residues in a candidate sequence that are identical with the amino acid residues in the reference polypeptide sequence, such as a lysin polypeptide sequence, after aligning the sequences and introducing gaps, if necessary, to achieve the maximum percent sequence identity, and not considering any conservative substitutions as a part of the sequence identity. Alignment for purposes of determining percent amino acid sequence identity can be achieved in various ways that are within the skill in the art, for example, using publicly available software such as BLAST or software available commercially for example from DNASTAR. Two or more polypeptide sequences can be anywhere from 0-100% identical, or any integer value there between. In the context of the present disclosure, two polypeptides are “substantially identical” when at least 80% of the amino acid residues (preferably at least about 85%, at least about 90%, and preferably at least about 95%, at least about 98%, or at least 99%) are identical. The term “percent (%) amino acid sequence identity” as described herein applies to peptides as well. Thus, the term “Substantially identical” will encompass mutated, truncated, fused, or otherwise sequence-modified variants of isolated polypeptides and peptides, such as those described herein, and active fragments thereof, as well as polypeptides with substantial sequence identity (e.g., at least 80%, at least 85%, at least 90%, at least 95% identity, at least 98% identity, or at least 99% identity as measured for example by one or more methods referenced above) as compared to the reference (wild type or other intact) polypeptide. Two amino acid sequences are “substantially homologous” when at least about 80% of the amino acid residues (preferably at least about 85%, at least about 90%, at least about 95%, at least about 98% identity, or at least about 99% identity) are identical, or represent conservative substitutions. The sequences of polypeptides of the present disclosure, are substantially homologous when one or more, or several, or up to 10%, or up to 15%, or up to 20% of the amino acids of the polypeptide, such as the lysin and/or fusion polypeptides described herein, are substituted with a similar or conservative amino acid substitution, and wherein the resulting polypeptide, such as the lysin and/or fusion polypeptides described herein, have at least one activity, antibacterial effects, and/or bacterial specificities of the reference polypeptide, such as the lysin and/or fusion polypeptides described herein.

As used herein, a “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

“Inhalable composition” refers to pharmaceutical compositions of the present disclosure that are formulated for direct delivery to the respiratory tract during or in conjunction with routine or assisted respiration (e.g., by intratracheobronchial, pulmonary, and/or nasal administration), including, but not limited to, atomized, nebulized, dry powder, and/or aerosolized formulations.

“Biofilm” refers to bacteria that attach to surfaces and aggregate in a hydrated polymeric matrix that may be comprised of bacterial- and/or host-derived components. A biofilm is an aggregate of microorganisms in which cells adhere to each other on a biotic or abiotic surface. These adherent cells are frequently embedded within a matrix comprised of, but not limited to, extracellular polymeric substance (EPS). Biofilm EPS, which is also referred to as slime (although not everything described as slime is a biofilm) or plaque, is a polymeric conglomeration generally composed of extracellular DNA, proteins, and polysaccharides. In certain embodiments, the biofilm may contain Staphylococcus and/or Streptococcus bacteria.

“Suitable” in the context of an antibiotic being suitable for use against certain bacteria refers to an antibiotic that was found to be effective against those bacteria even if resistance subsequently developed.

“Wild-type PlySs2 lysin” and “PlySs2 lysin,” refer to a polypeptide having the amino acid sequence:

MTTVNEALNNVRAQVGSGVSVGNGECYALASWYERMISPDATVGLGAG VGWVSGAIGDTISAKNIGSSYNWQANGWTVSTSGPFKAGQIVTLGATPGNPYGHVVI VEAVDGDRLTILEQNYGGKRYPVRNYYSAASYRQQVVHYITPPGTVAQSAPNLAGS RSYRETGTMTVTVDALNVRRAPNTSGEIVAVYKRGESFDYDTVIIDVNGYVWVSYIG GSGKRNYVATGATKDGKRFGNAWGTFK (SEQ ID NO: 1; 245 amino acid residues including the initial methionine residue which is removed during post-translational processing, leaving a 244-amino acid peptide).

“Modified lysin polypeptide” as used herein refers to a non-naturally occurring variant (or active fragment thereof) of the wild-type PlySs2 lysin. The modified lysin polypeptide has at least one amino acid substitution in the CHAP domain and/or the SH3b domain, and inhibits the growth, reduces the population, or kills at least one species of Gram-positive bacteria, such as S. aureus.

“Immunogenic” or “immunogenicity” means predicted to be immunogenic or to have immunogenicity by establishing (for example, through computationally guided in silico methods) the existence of one or more T-cell epitopes Immunogenicity of a modified lysin polypeptide as disclosed herein can be measured by TCE score, using any available in silico computationally guided method for obtaining such score and compared to the similarly derived TCE score of a wild-type PlySs2 lysin having the amino acid sequence of SEQ ID NO: 1. Alternatively, immunogenicity of a modified lysin polypeptide as disclosed herein can be measured by an in vitro T cell response. By extension, “less immunogenic,” “reduced immunogenicity,” or the like means predicted to be less immunogenic or to have reduced immunogenicity by depletion (which includes elimination or attenuation by amino acid replacement) of one or more T-cell epitopes (i.e., have a lower TCE score as compared to a reference polypeptide) or that the modified lysin polypeptide as disclosed herein elicits a reduced T cell response. Therefore, as used herein, a modified lysin polypeptide is “less immunogenic,” or has “reduced immunogenicity,” or the like if the modified lysin polypeptide has either 1) a lower TCE score than a wild-type PlySs2 lysin having the amino acid sequence of SEQ ID NO: 1 or 2) a reduced T cell response.

“Reduced T cell response” means that the modified lysin polypeptide induces less T cell activation than a wild-type PlySs2 lysin having the amino acid sequence of SEQ ID NO: 1, as measured by an in vitro T cell proliferation (³{H}-thymidine incorporation) assay using CD8+ depleted, human peripheral blood mononuclear cells in which the human peripheral blood mononuclear cells are exposed to fluorescein isothiocyanate-labeled anti-cytokine antibodies and the response measured.

“Substantially” used in the context of lytic activity (antimicrobial activity) of a modified lysin polypeptide of the present disclosure means at least a considerable portion of the antibacterial activity of the wild-type PlySs2 lysin, such that, on the basis of such activity, the modified lysin polypeptide would be useful alone or together with other antimicrobial agents, such as one or more antibiotics and/or lysostaphin, to inhibit, combat, or eliminate Staphylococcal or Streptococcal bacterial infection by killing these bacteria. Nonlimiting examples of such substantial activity compared to the wild-type PlySs2 lysin include no more than about 5, such as no more than about 4, no more than about 3, or no more than about 2, times the MIC of the wild-type lysin. Other measures of activity can be, for example, minimum biofilm eliminating concentration (MBEC) or in vivo efficacy using, for example, an animal model, such as the mouse neutropenic thigh infection model (MNTI). Still other measures can be the ability to synergize with antibiotics, such as vancomycin or daptomycin, or the ability to ameliorate, prevent, or delay development of, bacterial resistance of antibiotics, such as vancomycin or daptomycin, similar to the wild-type PlySs2 lysin. The same term “substantially” used in the context of reduced immunogenicity means having at most 65%, such as at most 50%, at most 40%, at most 30%, or at most 25% of the immunogenicity of the wild-type PlySs2 lysin, as measured for example by a TCE score [19].

### Modified Lysin Polypeptides

In one aspect, the present disclosure is directed to a modified lysin polypeptide having lytic activity and reduced immunogenicity as compared to a wild-type PlySs2 lysin. As used herein “lytic activity” encompasses the ability of a lysin to kill bacteria, reduce the population of bacteria or inhibit bacterial growth. Lytic activity also encompasses the ability to remove or reduce a biofilm and/or the ability to reduce the minimum inhibitory concentration (MIC) of an antibiotic.

Typically, the present modified lysin polypeptides are capable of degrading peptidoglycan, a major structural component of the bacterial cell wall, resulting in cell lysis. The modified lysin polypeptides are further capable of reducing immunogenicity and/or reducing inflammatory response-related toxicity compared to a wild-type PlySs2 lysin.

Suitable methods for assessing the activity of a modified lysin polypeptide as disclosed herein are well known in the art and described in the examples. Briefly, a MIC value (i.e., the minimum concentration of peptide sufficient to suppress at least 80% of the bacterial growth compared to control) may be determined for a modified lysin polypeptide and compared to, e.g., a wild-type PlySs2 lysin or inactive compound. For example, MIC values for a modified lysin polypeptide may be determined against e.g., laboratory Staphylococcus aureus strains, in e.g., Mueller-Hinton broth or Mueller-Hinton broth supplemented with serum, such as horse serum.

In some embodiments, the present modified lysin polypeptides are capable of reducing a biofilm. Methods for assessing the Minimal Biofilm Eradicating Concentration (MBEC) of a modified lysin polypeptide may be determined using a variation of the broth microdilution MIC method with modifications (See Ceri et al. 1999. J. Clin Microbial. 37:1771-1776, which is herein incorporated by reference in its entirety and Schuch et al., 2017, Antimicrob. Agents Chemother. 61, pages 1-18, which is herein incorporated by reference in its entirety.) In this method, colonies of bacteria, e.g., Staphylococcus aureus such as methicillin-resistant S. aureus (MRSA) and methicillin-susceptible S. aureus (MSSA), are suspended in medium, e.g., phosphate buffer solution (PBS) diluted e.g., 1:100 in TSBg (tryptic soy broth supplemented with 0.2% glucose), added as e.g., 0.15 ml aliquots, to a Calgary Biofilm Device (96-well plate with a lid bearing 96 polycarbonate pegs; Innovotech Inc.) and incubated e.g., 24 hours at 37° C. Biofilms are then washed and treated with e.g., a 2-fold dilution series of the lysin in TSBg at e.g., 37° C. for 24 hours. After treatment, wells are washed, air-dried at e.g., 37° C. and stained with e.g., 0.05% crystal violet for 10 minutes. After staining, the biofilms are destained in e.g., 33% acetic acid and the OD600 of e.g., extracted crystal violet is determined. The MBEC of each sample is the minimum lysin concentration required to remove >95% of the biofilm biomass assessed by crystal violet quantitation.

In some embodiments, the present modified lysin polypeptides reduce the minimum inhibitory concentration (MIC) of an antibiotic. Any known method to assess MIC may be used. In some embodiments, a checkerboard assay is used to determine the effect of a lysin on antibiotic concentration. The checkerboard assay is based on a modification of the CLSI method for MIC determination by broth microdilution (See Clinical and Laboratory Standards Institute (CLSI), CLSI. 2015. Methods for Dilution Antimicrobial Susceptibility Tests for Bacteria That Grow Aerobically; Approved Standard-10th Edition. Clinical and Laboratory Standards Institute, Wayne, Pa., which is herein incorporated by reference in its entirety and Ceri et al. 1999. J. Clin. Microbiol. 37: 1771-1776, which is also herein incorporated by reference in its entirety).

Checkerboards are constructed by first preparing columns of e.g., a 96-well polypropylene microtiter plate, wherein each well has the same amount of antibiotic diluted 2-fold along the horizontal axis. In a separate plate, comparable rows are prepared in which each well has the same amount of lysin diluted e.g., 2-fold along the vertical axis. The lysin and antibiotic dilutions are then combined, so that each column has a constant amount of antibiotic and doubling dilutions of lysin, while each row has a constant amount of lysin and doubling dilutions of antibiotic. Each well thus has a unique combination of lysin and antibiotic. Bacteria are added to the drug combinations at a given concentration. The MIC of each drug, alone and in combination, is then recorded after e.g., 16 hours at 37° C. in ambient air. Summation fractional inhibitory concentrations (ΣFICs) are calculated for each drug and the minimum ΣFIC value (ΣFICmin) is used to determine the effect of the lysin/antibiotic combination.

The lysin polypeptides disclosed herein have been modified from a wild-type PlySs2 lysin. PlySs2 is a bacteriophage lysin that may be derived from Streptococcus suis bacteria. PlySs2 demonstrates broad killing activity against multiple bacteria, including Gram-positive bacteria, including Staphylococcus, Streptococcus, Enterococcus, and Listeria bacterial strains, including antibiotic-resistant Staphylococcus aureus, such as MRSA and vancomycin-resistant Staphylococcus aureus (VRSA). Wild-type PlySs2 has the following amino acid sequence:

(SEQ ID NO: 1)

[symbol]

[symbol]

[symbol]

[symbol]

[symbol]

[symbol]

[symbol]

[symbol]

PPGTVAQSAP

[symbol]

[symbol]

[symbol]

[symbol]

.

SEQ ID NO: 1 has 245 amino acid residues, including the initial methionine residue which is removed during post-translational processing, leaving a 244-amino acid polypeptide. The italicized amino acids indicates the CHAP domain (amino acids 1 to 146) and the dotted underline indicates the SH3b domain (amino acids 157 to 245). The naturally occurring linker between the two domains is PPGTVAQSAP (SEQ ID NO: 2).

As disclosed herein, wild-type PlySs2 comprises both a CHAP domain and a SH3b domain, each of which in turn comprises multiple T-cell epitopes (TCE). TCE 1, TCE 2, TCE 3, and TCE 4 are located in the CHAP domain, while TCE 5, TCE 6, TCE 7, and TCE 8 are located in the SH3b domain. TCE 1 corresponds to amino acid residues 32-45 of SEQ ID NO: 1. TCE 2 corresponds to amino acid residues 84-98 of SEQ ID NO: 1. TCE 3 corresponds to amino acid residues 100-112 of SEQ ID NO: 1. TCE 4 corresponds to amino acid residues 128-145 of SEQ ID NO: 1. TCE 5 corresponds to amino acid residues 164-170 of SEQ ID NO: 1. TCE 6 corresponds to amino acid residues 172-187 of SEQ ID NO: 1. TCE 7 corresponds to amino acid residues 189-201 of SEQ ID NO: 1, and TCE 8 corresponds to amino acid residues 204-221 of SEQ ID NO: 1.

In certain embodiments, the modified lysin polypeptide comprises at least one substitution as compared to the wild-type PlySs2 polypeptide (SEQ ID NO: 1), wherein the at least one substitution is in one or more of TCE 1, TCE 2, TCE 3, or TCE 4, wherein the modified lysin polypeptide or fragment thereof inhibits the growth, reduces the population, or kills at least one species of Gram-positive bacteria. In certain embodiments, the modified lysin polypeptide comprises at least one substitution as compared to the wild-type PlySs2 polypeptide (SEQ ID NO: 1), wherein the at least one substitution is in one or more of TCE 5, TCE 6, TCE 7, or TCE 8, wherein the modified lysin polypeptide or fragment thereof inhibits the growth, reduces the population, or kills at least one species of Gram-positive bacteria. In certain embodiments, the modified lysin polypeptide comprises at least a first substitution and at least a second substitution as compared to the wild-type PlySs2 polypeptide (SEQ ID NO: 1), wherein the at least the first substitution is in one or more of TCE 1, TCE 2, TCE 3, or TCE 4 and at least the second substitution is in one or more of TCE 5, TCE 6, TCE 7, or TCE 8, wherein the modified lysin polypeptide or fragment thereof inhibits the growth, reduces the population, or kills at least one species of Gram-positive bacteria. Typically, the modified lysin polypeptide has reduced immunogenicity as compared to a wild-type PlySs2 having the amino acid sequence of SEQ ID NO: 1.

In certain embodiments, the modified lysin polypeptide comprises at least two substitutions as compared to the wild-type PlySs2 polypeptide (SEQ ID NO: 1), wherein the at least two substitutions are in TCE 4. In certain embodiments, the modified lysin polypeptide comprises at least four substitutions as compared to the wild-type PlySs2 polypeptide (SEQ ID NO: 1), wherein at least one substitution is in TCE 2, at least one substitution is in TCE 3, and at least two substitutions are in TCE 4.

In certain embodiments, a modified lysin polypeptide as disclosed herein may result from modifying the amino acid sequence of SEQ ID NO: 1 by an amino acid substitution in the CHAP domain in at least one position selected from amino acid residue 35, 92, 104, 128, and 137 and/or an amino acid substitution in the SH3b domain in at least one position selected from amino acid residue 164, 184, 195, 198, 204, 206, 212, and 214. Accordingly, in certain embodiments, disclosed herein is a modified lysin polypeptide having at least one amino acid substitution as compared to the wild-type PlySs2 polypeptide (SEQ ID NO: 1), wherein the modified lysin polypeptide comprises at least one amino acid substitution in the CHAP domain in at least one position selected from amino acid residue 35, 92, 104, 128, and 137 of SEQ ID NO: 1 and/or at least one amino acid substitution in SH3b domain in at least one position selected from amino acid residue 164, 184, 195, 198, 204, 206, 212, and 214 of SEQ ID NO: 1, wherein the modified lysin polypeptide or fragment thereof inhibits the growth, reduces the population, or kills at least one species of Gram-positive bacteria. In certain embodiments, the modified lysin polypeptide comprises an amino acid substitution in amino acid residues of 92, 104, 128, and 137 of SEQ ID NO: 1. In certain embodiments, the modified lysin polypeptide comprises an amino acid substitution in amino acid residues 92, 104, 128, 137, 164, 184, and 198 of SEQ ID NO: 1. Typically, the modified lysin polypeptide has reduced immunogenicity as compared to a wild-type PlySs2 having the amino acid sequence of SEQ ID NO: 1.

In certain embodiments, the modified lysin polypeptide may contain at least 3 amino acid substitutions, such as at least 4, at least 5, at least 6, at least 7, at least 8, or at least 9 amino acid substitutions. In certain embodiments, the modified lysin polypeptide may contain 3-9 amino acid substitutions, such as 4-9, 5-9, 6-9, 7-9, 8-9, or 9 amino acid substitutions relative to SEQ ID NO: 1. In certain embodiments, the modified lysin polypeptide may comprise at least two, such as at least three or at least four, amino acid substitutions relative to SEQ ID NO: 1 in the CHAP domain, and in certain embodiments, the modified lysin polypeptide may comprise at least two, such as at least three or at least four, amino acid substitutions relative to SEQ ID NO: 1 in the SH3b domain. In certain embodiments, the modified lysin polypeptide may consist of two, three or four amino acid substitutions relative to SEQ ID NO: 1 in the CHAP domain, and in certain embodiments, the modified lysin polypeptide may consist of two, three, or four amino acid substitutions relative to SEQ ID NO: 1 in the SH3b domain.

In certain embodiments, the modified lysin polypeptide comprises one or more of the following amino acid substitutions relative to SEQ ID NO: 1: R35E, L92W, V104S, V128T, Y137S, Y164N, Y164K, N184D, R195E, S198H, S198Q, V204K, V204A, 1206E, V212E, V212A, and V214G. In certain embodiments, the modified lysin polypeptide comprises one or more of the following amino acid substitutions located in the CHAP domain: R35E, L92W, V104S, V128T and Y137S, and/or one or more of the following amino acid substitutions located in the SH3b domain: Y164N, Y164K, N184D, R195E, S198H, S198Q, V204K, V204A, 1206E, V212A, V212E, and V214G, wherein the modified lysin polypeptide or fragment thereof inhibits the growth, reduces the population, or kills at least one species of Gram-positive bacteria. Typically, the modified lysin polypeptide has reduced immunogenicity as compared to a wild-type PlySs2 having the amino acid sequence of SEQ ID NO: 1.

The substitutions herein are designated using the one-letter amino acid code of the original amino acid in SEQ ID NO: 1 that is replaced, followed by the amino acid position in SEQ ID NO: 1, followed by the amino acid that is substituted into the sequence to result in the modified lysin polypeptide. Accordingly, by way of example, R35E indicates a substitution wherein the arginine at amino acid number 35 of SEQ ID NO: 1 is replaced with glutamic acid.

Exemplary modified lysin polypeptides are disclosed herein as pp55, pp61, pp65, pp296, pp324, pp325, pp341, pp338, pp388, pp400, pp616, pp619, pp628, pp632, and pp642.

The exemplary modified lysin polypeptides comprise the amino acid substitutions relative to the amino acid sequence of SEQ ID NO:1 as shown below in Table 1.

TABLE 1

| | Substitution location | | | | | | | |
|---|---|---|---|---|---|---|---|---|
| No. | TCE 1 | TCE 2 | TCE 3 | TCE 4 | TCE 5 | TCE 6 | TCE 7 | TCE 8 |
| pp55 | | L92W | V104S | V128T | | | | |
| | | | | and | | | | |
| | | | | Y137S | | | | |
| pp61 | | L92W | V104S | V128T | | | S198H | I206E |
| | | | | and | | | | |
| | | | | Y137S | | | | |
| pp65 | | L92W | V104S | V128T | | | S198Q | V204A |
| | | | | and | | | | and |
| | | | | Y137S | | | | V212A |
| pp296 | | L92W | V104S | V128T | Y164K | N184D | S198Q | |
| | | | | and | | | | |
| | | | | Y137S | | | | |
| pp324 | | L92W | V104S | V128T | Y164N | N184D | | |
| | | | | and | | | | |
| | | | | Y137S | | | | |
| pp325 | | L92W | V104S | V128T | Y164N | | R195E | |
| | | | | and | | | | |
| | | | | Y137S | | | | |
| pp341 | | L92W | V104S | V128T | | N184D | | V204A |
| | | | | and | | | | and |
| | | | | Y137S | | | | V212A |
| pp338 | | L92W | V104S | V128T | | N184D | S198H | |
| | | | | and | | | | |
| | | | | Y137S | | | | |
| pp388 | | | | | Y164N | N184D | R195E | V204K |
| | | | | | | | | and |
| | | | | | | | | V212E |
| pp400 | R35E | L92W | V104S | V128T | | | | |
| | | | | and | | | | |
| | | | | Y137S | | | | |
| pp616 | | | | V128T | Y164K | | | |
| | | | | and | | | | |
| | | | | Y137S | | | | |
| pp619 | | L92W | V104S | V128T | Y164K | | | |
| | | | | and | | | | |
| | | | | Y137S | | | | |
| pp628 | | L92W | V104S | V128T | Y164K | | | V204K |
| | | | | and | | | | and |
| | | | | Y137S | | | | V212E |
| pp632 | | L92W | V104S | V128T | Y164K | N184D | S198Q | V204K |
| | | | | and | | | | and |
| | | | | Y137S | | | | V212E |
| pp642 | | L92W | V104S | V128T | Y164K | | | I206E |
| | | | | and | | | | and |
| | | | | Y137S | | | | V214G |

In certain embodiments disclosed herein, the modified lysin polypeptide is pp55 and comprises the following amino acid substitutions relative to the amino acid sequence of SEQ ID NO: 1: L92W, V104S, V128T, and Y137S. In certain embodiments, the modified lysin polypeptide comprises the amino acid sequence of SEQ ID NO: 3. In certain embodiments, the modified lysin polypeptide has at least 80% sequence identity with SEQ ID NO: 3, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the modified lysin polypeptide has at least 85% sequence identity with SEQ ID NO: 3. In certain embodiments, the modified lysin polypeptide has at least 90% sequence identity with SEQ ID NO: 3. In certain embodiments, the modified lysin polypeptide has at least 95% sequence identity with SEQ ID NO: 3. In certain embodiments, the modified lysin polypeptide has at least 98% sequence identity with SEQ ID NO: 3. In certain embodiments, the modified lysin polypeptide has at least 99% sequence identity with SEQ ID NO: 3.

In certain embodiments disclosed herein, the modified lysin polypeptide is pp61 and comprises the following amino acid substitutions relative to the amino acid sequence of SEQ ID NO: 1: L92W, V104S, V128T, Y137S, S198H, and 1206E. In certain embodiments, the modified lysin polypeptide comprises the amino acid sequence of SEQ ID NO: 4, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the modified lysin polypeptide has at least 80% sequence identity with SEQ ID NO: 4. In certain embodiments, the modified lysin polypeptide has at least 85% sequence identity with SEQ ID NO: 4. In certain embodiments, the modified lysin polypeptide has at least 90% sequence identity with SEQ ID NO: 4. In certain embodiments, the modified lysin polypeptide has at least 95% sequence identity with SEQ ID NO: 4. In certain embodiments, the modified lysin polypeptide has at least 98% sequence identity with SEQ ID NO: 4. In certain embodiments, the modified lysin polypeptide has at least 99% sequence identity with SEQ ID NO: 4.

In certain embodiments disclosed herein, the modified lysin polypeptide thereof is pp65 and comprises the following amino acid substitutions relative to the amino acid sequence of SEQ ID NO: 1: L92W, V104S, V128T, Y137S, S198Q, V204A, and V212A. In certain embodiments, the modified lysin polypeptide comprises the amino acid sequence of SEQ ID NO: 5. In certain embodiments, the modified lysin polypeptide has at least 80% sequence identity with SEQ ID NO: 5, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the modified lysin polypeptide has at least 85% sequence identity with SEQ ID NO: 5. In certain embodiments, the modified lysin polypeptide has at least 90% sequence identity with SEQ ID NO: 5. In certain embodiments, the modified lysin polypeptide has at least 95% sequence identity with SEQ ID NO: 5. In certain embodiments, the modified lysin polypeptide has at least 98% sequence identity with SEQ ID NO: 5. In certain embodiments, the modified lysin polypeptide has at least 99% sequence identity with SEQ ID NO: 5.

In certain embodiments disclosed herein, the modified lysin polypeptide is pp296 and comprises the following amino acid substitutions relative to the amino acid sequence of SEQ ID NO: 1: L92W, V104S, V128T, Y137S, Y164K, N184D, and S198Q. In certain embodiments, the modified lysin polypeptide comprises the amino acid sequence of SEQ ID NO: 6. In certain embodiments, the modified lysin polypeptide has at least 80% sequence identity with SEQ ID NO: 6, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the modified lysin polypeptide has at least 85% sequence identity with SEQ ID NO: 6. In certain embodiments, the modified lysin polypeptide has at least 90% sequence identity with SEQ ID NO: 6. In certain embodiments, the modified lysin polypeptide has at least 95% sequence identity with SEQ ID NO: 6. In certain embodiments, the modified lysin polypeptide has at least 98% sequence identity with SEQ ID NO: 6. In certain embodiments, the modified lysin polypeptide has at least 99% sequence identity with SEQ ID NO: 6.

In certain embodiments disclosed herein, the modified lysin polypeptide is pp324 and comprises the following amino acid substitutions relative to the amino acid sequence of SEQ ID NO: 1: L92W, V104S, V128T, Y137S, Y164K, and N184D. In certain embodiments, the modified lysin polypeptide comprises the amino acid sequence of SEQ ID NO: 7. In certain embodiments, the modified lysin polypeptide has at least 80% sequence identity with SEQ ID NO: 7, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the modified lysin polypeptide has at least 85% sequence identity with SEQ ID NO: 7. In certain embodiments, the modified lysin polypeptide has at least 90% sequence identity with SEQ ID NO: 7. In certain embodiments, the modified lysin polypeptide has at least 95% sequence identity with SEQ ID NO: 7. In certain embodiments, the modified lysin polypeptide has at least 98% sequence identity with SEQ ID NO: 7. In certain embodiments, the modified lysin polypeptide has at least 99% sequence identity with SEQ ID NO: 7.

In certain embodiments disclosed herein, the modified lysin polypeptide is pp325 and comprises the following amino acid substitutions relative to the amino acid sequence of SEQ ID NO: 1: L92W, V104S, V128T, Y137S, Y164N, and R195E. In certain embodiments, the modified lysin polypeptide comprises the amino acid sequence of SEQ ID NO: 8. In certain embodiments, the modified lysin polypeptide has at least 80% sequence identity with SEQ ID NO: 8, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the modified lysin polypeptide has at least 85% sequence identity with SEQ ID NO: 8. In certain embodiments, the modified lysin polypeptide has at least 90% sequence identity with SEQ ID NO: 8. In certain embodiments, the modified lysin polypeptide has at least 95% sequence identity with SEQ ID NO: 8. In certain embodiments, the modified lysin polypeptide has at least 98% sequence identity with SEQ ID NO: 8. In certain embodiments, the modified lysin polypeptide has at least 99% sequence identity with SEQ ID NO: 8.

In certain embodiments disclosed herein, the modified lysin polypeptide is pp381 and comprises the following amino acid substitutions relative to the amino acid sequence of SEQ ID NO: 1: L92W, V104S, V128T, Y137S, N184D, and S198H. In certain embodiments, the modified lysin polypeptide comprises the amino acid sequence of SEQ ID NO: 9. In certain embodiments, the modified lysin polypeptide has at least 80% sequence identity with SEQ ID NO: 9, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the modified lysin polypeptide has at least 85% sequence identity with SEQ ID NO: 9. In certain embodiments, the modified lysin polypeptide has at least 90% sequence identity with SEQ ID NO: 9. In certain embodiments, the modified lysin polypeptide has at least 95% sequence identity with SEQ ID NO: 9. In certain embodiments, the modified lysin polypeptide has at least 98% sequence identity with SEQ ID NO: 9. In certain embodiments, the modified lysin polypeptide has at least 99% sequence identity with SEQ ID NO: 9.

In certain embodiments disclosed herein, the modified lysin polypeptide is pp341 and comprises the following amino acid substitutions relative to the amino acid sequence of SEQ ID NO: 1: L92W, V104S, V128T, Y137S, N184D, V204A, and V212A. In certain embodiments, the modified lysin polypeptide comprises the amino acid sequence of SEQ ID NO: 10, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the modified lysin polypeptide has at least 80% sequence identity with SEQ ID NO: 10. In certain embodiments, the modified lysin polypeptide has at least 85% sequence identity with SEQ ID NO: 10. In certain embodiments, the modified lysin polypeptide has at least 90% sequence identity with SEQ ID NO: 10. In certain embodiments, the modified lysin polypeptide has at least 95% sequence identity with SEQ ID NO: 10. In certain embodiments, the modified lysin polypeptide has at least 98% sequence identity with SEQ ID NO: 10. In certain embodiments, the modified lysin polypeptide has at least 99% sequence identity with SEQ ID NO: 10.

In certain embodiments disclosed herein, the modified lysin polypeptide is pp388 and comprises the following amino acid substitutions relative to the amino acid sequence of SEQ ID NO: 1: Y164N, N184D, R195E, V204K, and V212E. In certain embodiments, the modified lysin polypeptide comprises the amino acid sequence of SEQ ID NO: 11. In certain embodiments, the modified lysin polypeptide has at least 80% sequence identity with SEQ ID NO: 11, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the modified lysin polypeptide has at least 85% sequence identity with SEQ ID NO: 11. In certain embodiments, the modified lysin polypeptide has at least 90% sequence identity with SEQ ID NO: 11. In certain embodiments, the modified lysin polypeptide has at least 95% sequence identity with SEQ ID NO: 11. In certain embodiments, the modified lysin polypeptide has at least 98% sequence identity with SEQ ID NO: 11. In certain embodiments, the modified lysin polypeptide has at least 99% sequence identity with SEQ ID NO: 11.

In certain embodiments disclosed herein, the modified lysin polypeptide is pp400 and comprises the following amino acid substitutions relative to the amino acid sequence of SEQ ID NO: 1: R35E, L92W, V104S, V128T, and Y137S. In certain embodiments, the modified lysin polypeptide comprises the amino acid sequence of SEQ ID NO: 12. In certain embodiments, the modified lysin polypeptide has at least 80% sequence identity with SEQ ID NO: 12, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the modified lysin polypeptide has at least 85% sequence identity with SEQ ID NO: 12. In certain embodiments, the modified lysin polypeptide has at least 90% sequence identity with SEQ ID NO: 12. In certain embodiments, the modified lysin polypeptide has at least 95% sequence identity with SEQ ID NO: 12. In certain embodiments, the modified lysin polypeptide has at least 98% sequence identity with SEQ ID NO: 12. In certain embodiments, the modified lysin polypeptide has at least 99% sequence identity with SEQ ID NO: 12.

In certain embodiments disclosed herein, the modified lysin polypeptide is pp616 and comprises the following amino acid substitutions relative to the amino acid sequence of SEQ ID NO: 1: V128T, Y137S, and Y164K. In certain embodiments, the modified lysin polypeptide comprises the amino acid sequence of SEQ ID NO: 13. In certain embodiments, the modified lysin polypeptide has at least 80% sequence identity with SEQ ID NO: 13, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the modified lysin polypeptide has at least 85% sequence identity with SEQ ID NO: 13. In certain embodiments, the modified lysin polypeptide has at least 90% sequence identity with SEQ ID NO: 13. In certain embodiments, the modified lysin polypeptide has at least 95% sequence identity with SEQ ID NO: 13. In certain embodiments, the modified lysin polypeptide has at least 98% sequence identity with SEQ ID NO: 13. In certain embodiments, the modified lysin polypeptide has at least 99% sequence identity with SEQ ID NO: 13.

In certain embodiments disclosed herein, the modified lysin polypeptide is pp619 and comprises the following amino acid substitutions relative to the amino acid sequence of SEQ ID NO: 1: L92W, V104S, V128T, Y137S, and Y164K. In certain embodiments, the modified lysin polypeptide comprises the amino acid sequence of SEQ ID NO: 14. In certain embodiments, the modified lysin polypeptide has at least 80% sequence identity with SEQ ID NO: 14, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the modified lysin polypeptide has at least 85% sequence identity with SEQ ID NO: 14. In certain embodiments, the modified lysin polypeptide has at least 90% sequence identity with SEQ ID NO: 14. In certain embodiments, the modified lysin polypeptide has at least 95% sequence identity with SEQ ID NO: 14. In certain embodiments, the modified lysin polypeptide has at least 98% sequence identity with SEQ ID NO: 14. In certain embodiments, the modified lysin polypeptide has at least 99% sequence identity with SEQ ID NO: 14.

In certain embodiments disclosed herein, the modified lysin polypeptide is pp628 and comprises the following amino acid substitutions relative to the amino acid sequence of SEQ ID NO: 1: L92W, V104S, V128T, Y137S, Y164K, V204K, and V212E. In certain embodiments, the modified lysin polypeptide comprises the amino acid sequence of SEQ ID NO: 15. In certain embodiments, the modified lysin polypeptide has at least 80% sequence identity with SEQ ID NO: 15, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the modified lysin polypeptide has at least 85% sequence identity with SEQ ID NO: 15. In certain embodiments, the modified lysin polypeptide has at least 90% sequence identity with SEQ ID NO: 15. In certain embodiments, the modified lysin polypeptide has at least 95% sequence identity with SEQ ID NO: 15. In certain embodiments, the modified lysin polypeptide has at least 98% sequence identity with SEQ ID NO: 15. In certain embodiments, the modified lysin polypeptide has at least 99% sequence identity with SEQ ID NO: 15.

In certain embodiments disclosed herein, the modified lysin polypeptide is pp632 and comprises the following amino acid substitutions relative to the amino acid sequence of SEQ ID NO: 1: L92W, V104S, V128T, Y137S, Y164K, N184D, S198Q, V204K, and V212E. In certain embodiments, the modified lysin polypeptide comprises the amino acid sequence of SEQ ID NO: 16. In certain embodiments, the modified lysin polypeptide has at least 80% sequence identity with SEQ ID NO: 16, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the modified lysin polypeptide has at least 85% sequence identity with SEQ ID NO: 16. In certain embodiments, the modified lysin polypeptide has at least 90% sequence identity with SEQ ID NO: 16. In certain embodiments, the modified lysin polypeptide has at least 95% sequence identity with SEQ ID NO: 16. In certain embodiments, the modified lysin polypeptide has at least 98% sequence identity with SEQ ID NO: 16. In certain embodiments, the modified lysin polypeptide has at least 99% sequence identity with SEQ ID NO: 16.

In certain embodiments disclosed herein, the modified lysin polypeptide is pp642 and comprises the following amino acid substitutions relative to the amino acid sequence of SEQ ID NO: 1: L92W, V104S, V128T, Y137S, Y164K, 1206E, and V214G. In certain embodiments, the modified lysin polypeptide comprises the amino acid sequence of SEQ ID NO: 17. In certain embodiments, the modified lysin polypeptide has at least 80% sequence identity with SEQ ID NO: 17, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the modified lysin polypeptide has at least 85% sequence identity with SEQ ID NO: 17. In certain embodiments, the modified lysin polypeptide has at least 90% sequence identity with SEQ ID NO: 17. In certain embodiments, the modified lysin polypeptide has at least 95% sequence identity with SEQ ID NO: 17. In certain embodiments, the modified lysin polypeptide has at least 98% sequence identity with SEQ ID NO: 17. In certain embodiments, the modified lysin polypeptide has at least 99% sequence identity with SEQ ID NO: 17.

In addition to the at least one substitution in the CHAP and/or Ch3b domains, the modified lysin polypeptides can also include one or more amino acid insertions and/or deletions, provided those modifications do not interfere with the lytic activity and/or reduced immunogenicity of the modified lysin polypeptide.

Also disclosed are chimeric lysin polypeptides. Chimeric lysin polypeptides are known in the art. For example, ClyF is a chimeric lysin that combines the catalytic domain of Ply187 lysin (the N-terminal 157 amino acid residues) with the binding domain of PlySs2 (the C-terminal 99 residues) [10]. In certain embodiments, the chimeric lysin polypeptide comprises a modified PlySs2 CHAP domain, as disclosed herein, and the binding domain of another lysin. In certain embodiments, the chimeric lysin polypeptide comprises the catalytic domain of another lysin and a modified PlySs2 SH3b domain, as disclosed herein.

In some embodiments, an active fragment of the modified lysin polypeptide is obtained. The term “active fragment” refers to a portion of a full-length lysin, which retains one or more biological activities of the reference lysin. Thus, as used herein, an active fragment of a modified lysin polypeptides inhibits the growth, or reduces the population, or kills at least one Gram-positive bacterial species.

### Polynucleotides

In one aspect, the present disclosure is directed to an isolated polynucleotide comprising a nucleic acid molecule encoding a modified lysin polypeptide as disclosed herein, wherein the modified lysin polypeptide has lytic activity and reduced immunogenicity as compared to a wild-type PlySs2 lysin (SEQ ID NO: 1). In certain embodiments, the encoded modified lysin polypeptide or fragment thereof inhibits the growth, reduces the population, or kills at least one species of Gram-positive bacteria

In certain embodiments, the nucleic acid molecule encodes a modified lysin polypeptide, wherein the modified lysin polypeptide comprises at least one substitution in one or more of TCE 1, TCE 2, TCE 3, or TCE 4. In certain embodiments, the nucleic acid molecule encodes a modified lysin polypeptide, wherein the modified lysin polypeptide comprises at least one substitution in one or more of TCE 5, TCE 6, TCE 7, or TCE 8. In certain embodiments, the nucleic acid molecule encodes a modified lysin polypeptide, wherein the modified lysin polypeptide comprises at least a first substitution and at least a second substitution as compared to the wild-type PlySs2 polypeptide (SEQ ID NO: 1), wherein the at least the first substitution is in one or more of TCE 1, TCE 2, TCE 3, or TCE 4 and at least the second substitution is in one or more of TCE 5, TCE 6, TCE 7, or TCE 8. In certain embodiments, the nucleic acid molecule encodes a modified lysin polypeptide comprising at least two substitutions as compared to the wild-type PlySs2 polypeptide (SEQ ID NO: 1), wherein the at least two substitutions are in TCE4. In certain embodiments, the nucleic acid molecule encodes a modified lysin polypeptide comprising at least four substitutions as compared to the wild-type PlySs2 polypeptide (SEQ ID NO: 1), wherein at least one substitution is in TCE2, at least one substitution is in TCE3, and at least two substitutions are in TCE4.

In certain embodiments, the nucleic acid molecule encodes a modified lysin polypeptide, wherein the modified lysin polypeptide comprises at least one amino acid substitution as compared to the wild-type PlySs2 polypeptide (SEQ ID NO: 1), wherein the modified lysin polypeptide comprises at least one amino acid substitution in the CHAP domain in at least one position selected from amino acid residue 35, 92, 104, 128, and 137 of SEQ ID NO: 1 and/or at least one amino acid substitution in the SH3b domain in at least one position selected from amino acid residue 164, 184, 195, 198, 204, 206, 212, and 214 of SEQ ID NO: 1. In certain embodiments, the nucleic acid molecule encodes a modified lysin polypeptide, wherein the modified lysin polypeptide comprises an amino acid substitution in amino acid residues of 92, 104, 128, and 137 of SEQ ID NO: 1. In certain embodiments, the nucleic acid molecule encodes a modified lysin polypeptide, wherein the modified lysin polypeptide comprises an amino acid substitution in amino acid residues 92, 104, 128, 137, 164, 184, and 198 of SEQ ID NO: 1.

In certain embodiments, the nucleic acid molecule encodes a modified lysin polypeptide, wherein the modified lysin polypeptide comprises one or more of the following amino acid substitutions relative to SEQ ID NO: 1: R35E, L92W, V104S, V128T, Y137S, Y164N, Y164K, N184D, R195E, S198H, S198Q, V204K, V204A, 1206E, V212E, V212A, and V214G. In certain embodiments, the nucleic acid molecule encodes a modified lysin polypeptide, wherein the modified lysin polypeptide comprises one or more of the following amino acid substitutions located in the CHAP domain: R35E, L92W, V104S, V128T and Y137S, and/or one or more of the following amino acid substitutions located in the SH3b domain: Y164N, Y164K, N184D, R195E, S198H, S198Q, V204K, V204A, I206E, V212A, V212E, and V214G.

In certain embodiments, the nucleic acid molecule encodes a modified lysin polypeptide, wherein the modified lysin polypeptide comprises the following amino acid substitutions relative to SEQ ID NO: 1: L92W, V104S, V128T, and Y137S. In certain embodiments, the nucleic acid molecule encodes a modified lysin polypeptide, wherein the modified lysin polypeptide comprises the amino acid sequence of SEQ ID NO: 3. In certain embodiments, the nucleic acid molecule encodes a modified lysin polypeptide having at least 80% sequence identity with SEQ ID NO: 3, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85% sequence identity with SEQ ID NO: 3. In certain embodiments, the encoded modified lysin polypeptide has at least 90% sequence identity with SEQ ID NO: 3. In certain embodiments, the encoded modified lysin polypeptide has at least 95% sequence identity with SEQ ID NO: 3. In certain embodiments, the encoded modified lysin polypeptide has at least 98% sequence identity with SEQ ID NO: 3. In certain embodiments, the encoded modified lysin polypeptide has at least 99% sequence identity with SEQ ID NO: 3.

In certain embodiments, the nucleic acid molecule encodes a modified lysin polypeptide, wherein the modified lysin polypeptide comprises the following amino acid substitutions relative to SEQ ID NO: 1: L92W, V104S, V128T, Y137S, S198H, and I206E. In certain embodiments, the nucleic acid molecule encodes a modified lysin polypeptide, wherein the modified lysin polypeptide comprises the amino acid sequence of SEQ ID NO: 4. In certain embodiments, the nucleic acid molecule encodes a modified lysin polypeptide having at least 80% sequence identity with SEQ ID NO: 4, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85% sequence identity with SEQ ID NO: 4. In certain embodiments, the encoded modified lysin polypeptide has at least 90% sequence identity with SEQ ID NO: 4. In certain embodiments, the encoded modified lysin polypeptide has at least 95% sequence identity with SEQ ID NO: 4. In certain embodiments, the encoded modified lysin polypeptide has at least 98% sequence identity with SEQ ID NO: 4. In certain embodiments, the encoded modified lysin polypeptide has at least 99% sequence identity with SEQ ID NO: 4.

In certain embodiments, the nucleic acid molecule encodes a modified lysin polypeptide, wherein the modified lysin polypeptide comprises the following amino acid substitutions relative to SEQ ID NO: 1: L92W, V104S, V128T, Y137S, S198Q, V204A, and V212A. In certain embodiments, the nucleic acid molecule encodes a modified lysin polypeptide, wherein the modified lysin polypeptide comprises the amino acid sequence of SEQ ID NO: 5. In certain embodiments, the nucleic acid molecule encodes a modified lysin polypeptide having at least 80% sequence identity with SEQ ID NO: 5, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85% sequence identity with SEQ ID NO: 5. In certain embodiments, the encoded modified lysin polypeptide has at least 90% sequence identity with SEQ ID NO: 5. In certain embodiments, the encoded modified lysin polypeptide has at least 95% sequence identity with SEQ ID NO: 5. In certain embodiments, the encoded modified lysin polypeptide has at least 98% sequence identity with SEQ ID NO: 5. In certain embodiments, the encoded modified lysin polypeptide has at least 99% sequence identity with SEQ ID NO: 5.

In certain embodiments, the nucleic acid molecule encodes a modified lysin polypeptide, wherein the modified lysin polypeptide comprises the following amino acid substitutions relative to SEQ ID NO: 1: L92W, V104S, V128T, Y137S, Y164K, N184D, and S198Q. In certain embodiments, the nucleic acid molecule encodes a modified lysin polypeptide, wherein the modified lysin polypeptide comprises the amino acid sequence of SEQ ID NO: 6. In certain embodiments, the nucleic acid molecule encodes a modified lysin polypeptide having at least 80% sequence identity with SEQ ID NO: 6, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85% sequence identity with SEQ ID NO: 6. In certain embodiments, the encoded modified lysin polypeptide has at least 90% sequence identity with SEQ ID NO: 6. In certain embodiments, the encoded modified lysin polypeptide has at least 95% sequence identity with SEQ ID NO: 6. In certain embodiments, the encoded modified lysin polypeptide has at least 98% sequence identity with SEQ ID NO: 6. In certain embodiments, the encoded modified lysin polypeptide has at least 99% sequence identity with SEQ ID NO: 6.

In certain embodiments, the nucleic acid molecule encodes a modified lysin polypeptide, wherein the modified lysin polypeptide comprises the following amino acid substitutions relative to SEQ ID NO: 1: L92W, V104S, V128T, Y137S, Y164K, and N184D. In certain embodiments, the nucleic acid molecule encodes a modified lysin polypeptide, wherein the modified lysin polypeptide comprises the amino acid sequence of SEQ ID NO: 7. In certain embodiments, the nucleic acid molecule encodes a modified lysin polypeptide having at least 80% sequence identity with SEQ ID NO: 7, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85% sequence identity with SEQ ID NO: 7. In certain embodiments, the encoded modified lysin polypeptide has at least 90% sequence identity with SEQ ID NO: 7. In certain embodiments, the encoded modified lysin polypeptide has at least 95% sequence identity with SEQ ID NO: 7. In certain embodiments, the encoded modified lysin polypeptide has at least 98% sequence identity with SEQ ID NO: 7. In certain embodiments, the encoded modified lysin polypeptide has at least 99% sequence identity with SEQ ID NO: 7.

In certain embodiments, the nucleic acid molecule encodes a modified lysin polypeptide, wherein the modified lysin polypeptide comprises the following amino acid substitutions relative to SEQ ID NO: 1: L92W, V104S, V128T, Y137S, Y164N, and R195E. In certain embodiments, the nucleic acid molecule encodes a modified lysin polypeptide, wherein the modified lysin polypeptide comprises the amino acid sequence of SEQ ID NO: 8. In certain embodiments, the nucleic acid molecule encodes a modified lysin polypeptide having at least 80% sequence identity with SEQ ID NO: 8, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85% sequence identity with SEQ ID NO: 8. In certain embodiments, the encoded modified lysin polypeptide has at least 90% sequence identity with SEQ ID NO: 8. In certain embodiments, the encoded modified lysin polypeptide has at least 95% sequence identity with SEQ ID NO: 8. In certain embodiments, the encoded modified lysin polypeptide has at least 98% sequence identity with SEQ ID NO: 8. In certain embodiments, the encoded modified lysin polypeptide has at least 99% sequence identity with SEQ ID NO: 8.

In certain embodiments, the nucleic acid molecule encodes a modified lysin polypeptide, wherein the modified lysin polypeptide comprises the following amino acid substitutions relative to SEQ ID NO: 1: L92W, V104S, V128T, Y137S, N184D, and S198H. In certain embodiments, the nucleic acid molecule encodes a modified lysin polypeptide, wherein the modified lysin polypeptide comprises the amino acid sequence of SEQ ID NO: 9. In certain embodiments, the nucleic acid molecule encodes a modified lysin polypeptide having at least 80% sequence identity with SEQ ID NO: 9, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85% sequence identity with SEQ ID NO: 9. In certain embodiments, the encoded modified lysin polypeptide has at least 90% sequence identity with SEQ ID NO: 9. In certain embodiments, the encoded modified lysin polypeptide has at least 95% sequence identity with SEQ ID NO: 9. In certain embodiments, the encoded modified lysin polypeptide has at least 98% sequence identity with SEQ ID NO: 9. In certain embodiments, the encoded modified lysin polypeptide has at least 99% sequence identity with SEQ ID NO: 9.

In certain embodiments, the nucleic acid molecule encodes a modified lysin polypeptide, wherein the modified lysin polypeptide comprises the following amino acid substitutions relative to SEQ ID NO: 1: L92W, V104S, V128T, Y137S, N184D, V204A, and V212A. In certain embodiments, the nucleic acid molecule encodes a modified lysin polypeptide, wherein the modified lysin polypeptide comprises the amino acid sequence of SEQ ID NO: 10. In certain embodiments, the nucleic acid molecule encodes a modified lysin polypeptide having at least 80% sequence identity with SEQ ID NO: 10, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85% sequence identity with SEQ ID NO: 10. In certain embodiments, the encoded modified lysin polypeptide has at least 90% sequence identity with SEQ ID NO: 10. In certain embodiments, the encoded modified lysin polypeptide has at least 95% sequence identity with SEQ ID NO: 10. In certain embodiments, the encoded modified lysin polypeptide has at least 98% sequence identity with SEQ ID NO: 10. In certain embodiments, the encoded modified lysin polypeptide has at least 99% sequence identity with SEQ ID NO: 10.

In certain embodiments, the nucleic acid molecule encodes a modified lysin polypeptide, wherein the modified lysin polypeptide comprises the following amino acid substitutions relative to SEQ ID NO: 1: Y164N, N184D, R195E, V204K, and V212E. In certain embodiments, the nucleic acid molecule encodes a modified lysin polypeptide, wherein the modified lysin polypeptide comprises the amino acid sequence of SEQ ID NO: 11. In certain embodiments, the nucleic acid molecule encodes a modified lysin polypeptide having at least 80% sequence identity with SEQ ID NO: 11, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85% sequence identity with SEQ ID NO: 11. In certain embodiments, encoded modified lysin polypeptide has at least 90% sequence identity with SEQ ID NO: 11. In certain embodiments, the encoded modified lysin polypeptide has at least 95% sequence identity with SEQ ID NO: 11. In certain embodiments, the encoded modified lysin polypeptide has at least 98% sequence identity with SEQ ID NO: 11. In certain embodiments, the encoded modified lysin polypeptide has at least 99% sequence identity with SEQ ID NO: 11.

In certain embodiments, the nucleic acid molecule encodes a modified lysin polypeptide, wherein the modified lysin polypeptide comprises the following amino acid substitutions relative to SEQ ID NO: 1: R35E, L92W, V104S, V128T, and Y137S. In certain embodiments, the nucleic acid molecule encodes a modified lysin polypeptide, wherein the modified lysin polypeptide comprises the amino acid sequence of SEQ ID NO: 12. In certain embodiments, the nucleic acid molecule encodes a modified lysin polypeptide having at least 80% sequence identity with SEQ ID NO: 12, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85% sequence identity with SEQ ID NO: 12. In certain embodiments, the encoded modified lysin polypeptide has at least 90% sequence identity with SEQ ID NO: 12. In certain embodiments, the encoded modified lysin polypeptide has at least 95% sequence identity with SEQ ID NO: 12. In certain embodiments, the encoded modified lysin polypeptide has at least 98% sequence identity with SEQ ID NO: 12. In certain embodiments, encoded modified lysin polypeptide has at least 99% sequence identity with SEQ ID NO: 12.

In certain embodiments, the nucleic acid molecule encodes a modified lysin polypeptide, wherein the modified lysin polypeptide comprises the following amino acid substitutions relative to SEQ ID NO: 1: V128T, Y137S, and Y164K. In certain embodiments, the nucleic acid molecule encodes a modified lysin polypeptide, wherein the modified lysin polypeptide comprises the amino acid sequence of SEQ ID NO: 13. In certain embodiments, the nucleic acid molecule encodes a modified lysin polypeptide having at least 80% sequence identity with SEQ ID NO: 13, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85% sequence identity with SEQ ID NO: 13. In certain embodiments, the encoded modified lysin polypeptide has at least 90% sequence identity with SEQ ID NO: 13. In certain embodiments, the encoded modified lysin polypeptide has at least 95% sequence identity with SEQ ID NO: 13. In certain embodiments, the encoded modified lysin polypeptide has at least 98% sequence identity with SEQ ID NO: 13. In certain embodiments, the encoded modified lysin polypeptide has at least 99% sequence identity with SEQ ID NO: 13.

In certain embodiments, the nucleic acid molecule encodes a modified lysin polypeptide, wherein the modified lysin polypeptide comprises the following amino acid substitutions relative to SEQ ID NO: 1: L92W, V104S, V128T, Y137S, and Y164K. In certain embodiments, the nucleic acid molecule encodes a modified lysin polypeptide, wherein the modified lysin polypeptide comprises the amino acid sequence of SEQ ID NO: 14. In certain embodiments, the nucleic acid molecule encodes a modified lysin polypeptide having at least 80% sequence identity with SEQ ID NO: 14, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85% sequence identity with SEQ ID NO: 14. In certain embodiments, the encoded modified lysin polypeptide has at least 90% sequence identity with SEQ ID NO: 14. In certain embodiments, the encoded modified lysin polypeptide has at least 95% sequence identity with SEQ ID NO: 14. In certain embodiments, the encoded modified lysin polypeptide has at least 98% sequence identity with SEQ ID NO: 14. In certain embodiments, the encoded modified lysin polypeptide has at least 99% sequence identity with SEQ ID NO: 14.

In certain embodiments, the nucleic acid molecule encodes a modified lysin polypeptide, wherein the modified lysin polypeptide comprises the following amino acid substitutions relative to SEQ ID NO: 1: L92W, V104S, V128T, Y137S, Y164K, V204K, and V212E. In certain embodiments, the nucleic acid molecule encodes a modified lysin polypeptide, wherein the modified lysin polypeptide comprises the amino acid sequence of SEQ ID NO: 15. In certain embodiments, the nucleic acid molecule encodes a modified lysin polypeptide having at least 80% sequence identity with SEQ ID NO: 15, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85% sequence identity with SEQ ID NO: 15. In certain embodiments, the encoded modified lysin polypeptide has at least 90% sequence identity with SEQ ID NO: 15. In certain embodiments, the encoded modified lysin polypeptide has at least 95% sequence identity with SEQ ID NO: 15. In certain embodiments, the encoded modified lysin polypeptide has at least 98% sequence identity with SEQ ID NO: 15. In certain embodiments, the encoded modified lysin polypeptide has at least 99% sequence identity with SEQ ID NO: 15.

In certain embodiments, the nucleic acid molecule encodes a modified lysin polypeptide, wherein the modified lysin polypeptide comprises the following amino acid substitutions relative to SEQ ID NO: 1: L92W, V104S, V128T, Y137S, Y164K, N184D, S198Q, V204K, and V212E. In certain embodiments, the nucleic acid molecule encodes a modified lysin polypeptide, wherein the modified lysin polypeptide comprises the amino acid sequence of SEQ ID NO: 16. In certain embodiments, the nucleic acid molecule encodes a modified lysin polypeptide having at least 80% sequence identity with SEQ ID NO: 16, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85% sequence identity with SEQ ID NO: 16. In certain embodiments, the encoded modified lysin polypeptide has at least 90% sequence identity with SEQ ID NO: 16. In certain embodiments, the encoded modified lysin polypeptide has at least 95% sequence identity with SEQ ID NO: 16. In certain embodiments, the encoded modified lysin polypeptide has at least 98% sequence identity with SEQ ID NO: 16. In certain embodiments, the encoded modified lysin polypeptide has at least 99% sequence identity with SEQ ID NO: 16.

In certain embodiments, the nucleic acid molecule encodes a modified lysin polypeptide, wherein the modified lysin polypeptide comprises the following amino acid substitutions relative to SEQ ID NO: 1: L92W, V104S, V128T, Y137S, Y164K, 1206E, and V214G. In certain embodiments, the nucleic acid molecule encodes a modified lysin polypeptide, wherein the modified lysin polypeptide comprises the amino acid sequence of SEQ ID NO: 17. In certain embodiments, the nucleic acid molecule encodes a modified lysin polypeptide having at least 80% sequence identity with SEQ ID NO: 17, wherein the modified lysin polypeptide inhibits the growth, reduces the population, or kills at least one species of Gram-positive bacteria and optionally wherein the modified lysin polypeptide has reduced immunogenicity as compared to the wild-type PlySs2 (SEQ ID NO: 1). In certain embodiments, the encoded modified lysin polypeptide has at least 85% sequence identity with SEQ ID NO: 17. In certain embodiments, the encoded modified lysin polypeptide has at least 90% sequence identity with SEQ ID NO: 17. In certain embodiments, the encoded modified lysin polypeptide has at least 95% sequence identity with SEQ ID NO: 17. In certain embodiments, the encoded modified lysin polypeptide has at least 98% sequence identity with SEQ ID NO: 17. In certain embodiments, the encoded modified lysin polypeptide has at least 99% sequence identity with SEQ ID NO: 17.

### Vectors and Host Cells

In another aspect, the present disclosure is directed to a vector comprising an isolated polynucleotide comprising a nucleic acid molecule encoding any of the modified lysin polypeptides disclosed herein or a complementary sequence of the present isolated polynucleotides. In some embodiments, the vector is a plasmid or cosmid. In other embodiments, the vector is a viral vector, wherein additional DNA segments can be ligated into the viral genome. In some embodiments, the vector can autonomously replicate in a host cell into which it is introduced. In some embodiments, the vector can be integrated into the genome of a host cell upon introduction into the host cell and thereby be replicated along with the host genome.

In some embodiments, particular vectors, referred to herein as “recombinant expression vectors” or “expression vectors,” can direct the expression of genes to which they are operatively linked. A polynucleotide sequence is “operatively linked” when it is placed into a functional relationship with another nucleotide sequence. For example, a promoter or regulatory DNA sequence is said to be “operatively linked” to a DNA sequence that codes for an RNA and/or a protein if the two sequences are operatively linked, or situated such that the promoter or regulatory DNA sequence affects the expression level of the coding or structural DNA sequence. Operatively linked DNA sequences are typically, but not necessarily, contiguous.

Generally, any system or vector suitable to maintain, propagate or express a polypeptide in a host may be used for expression of the modified lysin polypeptides disclosed herein or fragments thereof. The appropriate DNA/polynucleotide sequence may be inserted into the expression system by any of a variety of well-known and routine techniques, such as, for example, those set forth in Sambrook et al., eds., Molecular Cloning: A Laboratory Manual (3rd Ed.), Vols. 1-3, Cold Spring Harbor Laboratory (2001). Additionally, tags can also be added to the modified lysin polypeptides of the present disclosure to provide convenient methods of isolation, e.g., c-myc, biotin, poly-His, etc. Kits for such expression systems are commercially available.

A wide variety of host/expression vector combinations may be employed in expressing the polynucleotide sequences encoding the present modified lysin polypeptides. Large numbers of suitable vectors are known to those of skill in the art, and are commercially available. Examples of suitable vectors are provided, e.g., in Sambrook et al, eds., Molecular Cloning: A Laboratory Manual (3rd Ed.), Vols. 1-3, Cold Spring Harbor Laboratory (2001). Such vectors include, among others, chromosomal, episomal and virus derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids.

Furthermore, the vectors may provide for the constitutive or inducible expression of the modified lysin polypeptides of the present disclosure. Suitable vectors include but are not limited to derivatives of SV40 and known bacterial plasmids, e.g., E. coli plasmids colE1, pCR1, pBR322, pMB9 and their derivatives, plasmids such as RP4, pBAD24 and pBAD-TOPO; phage DNAS, e.g., the numerous derivatives of phage A, e.g., NM989, and other phage DNA, e.g., M13 and filamentous single stranded phage DNA; yeast plasmids such as the 2 D plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like. Many of the vectors mentioned above are commercially available from vendors such as New England Biolabs Inc., Addgene, Takara Bio Inc., ThermoFisher Scientific Inc., etc.

Additionally, vectors may comprise various regulatory elements (including promoter, ribosome binding site, terminator, enhancer, various cis-elements for controlling the expression level) wherein the vector is constructed in accordance with the host cell. Any of a wide variety of expression control sequences (sequences that control the expression of a polynucleotide sequence operatively linked to it) may be used in these vectors to express the polynucleotide sequences encoding the modified lysin polypeptides of the present disclosure. Useful control sequences include, but are not limited to: the early or late promoters of SV40, CMV, vaccinia, polyoma or adenovirus, the lac system, the trp system, the TAC system, the TRC system, the LTR system, the major operator and promoter regions of phage A, the control regions of fd coat protein, the promoter for 3-phosphoglycerate kinase or other glycolytic enzymes, the promoters of acid phosphatase (e.g., Pho5), the promoters of the yeast-mating factors, E. coli promoter for expression in bacteria, and other promoter sequences known to control the expression of genes of prokaryotic or eukaryotic cells or their viruses, and various combinations thereof. Typically, the polynucleotide sequences encoding the modified lysin polypeptides are operatively linked to a heterologous promoter or regulatory element.

In another aspect, the present disclosure is directed to an isolated host cell comprising any of the vectors disclosed herein including the expression vectors comprising the polynucleotide sequences encoding the modified lysin polypeptides of the present disclosure. A wide variety of host cells are useful in expressing the present polypeptides. Non-limiting examples of host cells suitable for expression of the present polypeptides include well known eukaryotic and prokaryotic hosts, such as strains of E. coli, Pseudomonas, Bacillus, Streptomyces, fungi such as yeasts, and animal cells, such as CHO, R1.1, B-W and L-M cells, African Green Monkey kidney cells (e.g., COS 1, COS 7, BSC1, BSC40, and BMT10), insect cells (e.g., Sf9), and human cells and plant cells in tissue culture.

While the expression host may be any known expression host cell, in a typical embodiment the expression host is one of the strains of E. coli. These include, but are not limited to commercially available E. coli strains such as Top10 (ThermoFisher Scientific, Inc.), DH5a (Thermo Fisher Scientific, Inc.), XLI-Blue (Agilent Technologies, Inc.), SCSllO (Agilent Technologies, Inc.), JM109 (Promega, Inc.), LMG194 (ATCC), and BL21 (Thermo Fisher Scientific, Inc.). There are several advantages of using E. coli as a host system including: fast growth kinetics, where under the optimal environmental conditions, its doubling time is about 20 min (Sezonov et al., J. Bacterial. 189 8746-8749 (2007)), easily achieved high density cultures, easy and fast transformation with exogenous DNA, etc. Details regarding protein expression in E. coli, including plasmid selection as well as strain selection are discussed in details by Rosano, G. and Ceccarelli, E., Front Microbial., 5: 172 (2014).

Efficient expression of the present modified lysin polypeptides depends on a variety of factors such as optimal expression signals (both at the level of transcription and translation), correct protein folding, and cell growth characteristics. Regarding methods for constructing the vector and methods for transducing the constructed recombinant vector into the host cell, conventional methods known in the art can be utilized. While it is understood that not all vectors, expression control sequences, and hosts will function equally well to express the polynucleotide sequences encoding the modified lysin polypeptides of the present disclosure, one skilled in the art will be able to select the proper vectors, expression control sequences, and hosts without undue experimentation to accomplish the desired expression without departing from the scope of this disclosure.

The modified lysin polypeptides of the present disclosure can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. High performance liquid chromatography can also employed for lysin polypeptide purification.

Alternatively, the vector system used for the production of the modified lysin polypeptides of the present disclosure may be a cell-free expression system. Various cell-free expression systems are commercially available, including, but are not limited to those available from Promega, LifeTechnologies, Clonetech, etc.

### Compositions Comprising Modified Lysin Polypeptides

The modified lysin polypeptides disclosed herein may be incorporated into antimicrobial and bactericidal compositions and unit dosage forms thereof alone or with one or more conventional antibiotics and other bactericidal agents.

Typically, the compositions contain the modified lysin polypeptide as disclosed herein in an amount effective for killing Gram-positive bacteria selected from the group consisting of Staphylococcus aureus; Listeria monocytogenes; a coagulase negative staphylococcus such as from the Staphylococcus epidermidis group, the Staphylococcus saprophyticus group, the Staphylococcus simulans group, the Staphylococcus intermedius group, the Staphylococcus sciuri group, and the Staphylococcus hyicus group; Streptococcus suis; Streptococcus pyogenes; Streptococcus agalactiae; Streptococcus dysgalactiae; Streptococcus pneumoniae; species included in the viridans streptococci group such as the Streptococcus anginosis group, Streptococcus mitis group, Streptococcus sanguinis group, Streptococcus bovis group, Streptococcus salivarius group, and Streptococcus mutans group; Enterococcus faecalis; and Enterococcus faecium.

The compositions disclosed herein can take the form of solutions, suspensions, emulsions, tablets, pills, pellets, capsules, capsules containing liquids, powders, sustained-release formulations, suppositories, tampon applications, aerosols, sprays, lozenges, troches, candies, injectables, chewing gums, ointments, smears, time-release patches, liquid-absorbed wipes, and combinations thereof. Hence, the compositions can be employed as solids, such as tablets, lyophilized powders for reconstitution, liposomes or micelles, or the compositions can be employed as liquids, such as solutions, suspensions, gargles, emulsions, or capsules filled solids or liquids, such as for oral use. In certain embodiments, the compositions can be in the form of suppositories or capsules for rectal administration or in the form of sterile injectable or inhalable solutions or suspensions for parenteral (including, for example, intravenous or subcutaneous) or topical, such as dermal, nasal, pharyngeal or pulmonary, use. Such compositions include pharmaceutical compositions, and unit dosage forms thereof may comprise conventional or new ingredients in conventional or special proportions, with or without additional active compounds or principles. Such unit dosage forms may contain any suitable effective amount of the active ingredient commensurate with the intended daily dosage range to be employed.

Carriers and excipients can be selected from a great variety of substances acceptable for human or veterinary use. Non-limiting examples of pharmaceutically acceptable carriers or excipients include any of the standard pharmaceutical carriers, such as phosphate buffered saline solutions, water, polyols, disaccharides or polysaccharides, and emulsions such as oil/water emulsions and microemulsions. Other stabilizing excipients include proprietary blends of stabilizing and protecting solutions (SPS), cyclodextrins and recombinant human albumin (rHSA). Other excipients may include bulking agents, buffering agents, tonicity modifiers (e.g., salts and amino acids), surfactants, preservatives, antioxidants, and co-solvents. For solid oral compositions comprising a modified lysin polypeptide disclosed herein, suitable pharmaceutically acceptable excipients include, but are not limited to, starches, sugars, diluents, granulating agents, lubricants, binders, disintegrating agents, and the like. For liquid oral compositions, suitable pharmaceutically acceptable excipients may include, but are not limited to, water, glycols, oils, alcohols, flavoring agents, preservatives, and the like. For topical solid compositions such as creams, gels, foams, ointments, or sprays, suitable excipients may include, but are not limited to a cream, a cellulosic, or an oily base, emulsifying agents, stiffening agents, rheology modifiers or thickeners, surfactants, emollients, preservatives, humectants, alkalizing or buffering agents, and solvents.

For example, the modified lysin polypeptides disclosed herein can be combined with buffers that maintain the pH of a liquid suspension, solution, or emulsion within a range that does not substantially affect the activity of the modified lysin polypeptide. For example, a desirable pH range of the composition or of the environment wherein the active ingredient is found upon administration may be between about 4.0 and about 9.0, for example between about 4.5 and about 8.5.

A stabilizing buffer may be optionally included to permit the modified lysin polypeptide to exert its activity in an optimized fashion. The buffer may contain a reducing reagent, such as dithiothreitol. The stabilizing buffer may also be or include a metal chelating reagent, such as ethylenediaminetetracetic acid disodium salt, or it may contain a phosphate or citrate-phosphate buffer, or any other buffering agent, such as Tris or succinate.

A mild surfactant can be included in a pharmaceutical composition in an amount effective to potentiate the therapeutic effect of the modified lysin polypeptides used in the composition. Suitable mild surfactants may include, inter alia, esters of polyoxyethylene sorbitan and fatty acids (such as the Tween series), octylphenoxy polyethoxy ethanol (such as the Triton-X series), n-Octyl-β-D-glucopyranoside, n-Octyl-β-D-thioglucopyranoside, n-Decyl-β-D-glucopyranoside, n-Dodecyl-β-D-glucopyranoside, poloxamer, polysorbate 20, polysorbate 80, polyethylene glycol, and biologically occurring surfactants, e.g., fatty acids, glycerides, monoglycerides, deoxycholate, and esters of deoxycholate.

Preservatives may also be used in the compositions disclosed herein, and may, for example, comprise about 0.05% to about 0.5% by weight of the total composition. The use of preservatives may assure that if the product is microbially-contaminated, the formulation will prevent or diminish microorganism growth (or attenuate the potency of the formulation). Exemplary preservatives include methylparaben, propylparaben, butylparaben, chloroxylenol, sodium benzoate, DMDM Hydantoin, 3-Iodo-2-Propylbutyl carbamate, potassium sorbate, chlorhexidine digluconate, or a combination thereof.

For oral administration, the modified lysin polypeptides disclosed herein can be formulated into solid or liquid preparations, for example tablets, capsules, powders, solutions, suspensions, and dispersions. For oral administration in the form of a tablet or capsule, the active ingredient may be combined with one or more pharmaceutically acceptable excipients such as binding agents (e.g., pregelatinized maize starch, polyvinylpyrrolidone, or hydroxypropyl methylcellulose); fillers (e.g., lactose, sucrose, glucose, mannitol, sorbitol, other reducing and non-reducing sugars, microcrystalline cellulose, calcium sulfate, or calcium hydrogen phosphate); lubricants (e.g., magnesium stearate, talc, silica, steric acid, sodium stearyl fumarate, glyceryl behenate, calcium stearate, and the like); disintegrants (e.g., potato starch or sodium starch glycolate); wetting agents (e.g., sodium lauryl sulphate), coloring and flavoring agents, gelatin, sweeteners, natural and synthetic gums (such as acacia, tragacanth or alginates), buffer salts, carboxymethylcellulose, polyethyleneglycol, waxes, and the like. For oral administration in liquid form, the drug components can be combined with non-toxic, pharmaceutically acceptable inert carriers (e.g., ethanol, glycerol, water), suspending agents (e.g., sorbitol syrup, cellulose derivatives or hydrogenated edible fats), emulsifying agents (e.g., lecithin or acacia), non-aqueous vehicles (e.g., almond oil, oily esters, ethyl alcohol or fractionated vegetable oils), preservatives (e.g., methyl or propyl-p-hydroxybenzoates or sorbic acid), and the like. Stabilizing agents such as antioxidants (e.g., BHA, BHT, propyl gallate, sodium ascorbate, or citric acid) can also be added to stabilize the dosage forms.

In certain embodiments, the tablets can be coated by methods well-known in the art. The compositions disclosed herein can be also introduced in microspheres or microcapsules, e.g., fabricated from polyglycolic acid/lactic acid (PGLA). Liquid preparations for oral administration can take the form of, for example, solutions, syrups, emulsions, or suspensions, or they can be presented as a dry product for reconstitution with water or other suitable vehicle before use. Preparations for oral administration can be suitably formulated to give controlled or postponed release of the active compound.

The active agents can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines, as is well known.

For preparing solid compositions such as tablets and pills, a modified lysin polypeptide as disclosed herein may be mixed with a pharmaceutical excipient to form a solid preformulation composition. If desired, tablets may be sugar coated or enteric coated by standard techniques. The tablets or pills may be coated or otherwise compounded to provide a dosage form affording the advantage of prolonged or delayed action. For example, the tablet or pill can include an inner dosage and an outer dosage component, the latter being in the form of an envelope over the former. The two components can be separated by an enteric layer, which serves to resist disintegration in the stomach and permit the inner component to pass intact into the duodenum or to be further delayed in release. A variety of materials can be used for such enteric layers or coatings, such materials including a number of polymeric acids and mixtures of polymeric acids with such materials as shellac, cetyl alcohol, and cellulose acetate. Similarly, the orally-administered medicaments may be administered in the form of a time-controlled release vehicle, including diffusion-controlled systems, osmotic devices, dissolution-controlled matrices, and erodible/degradable matrices.

Topical compositions as disclosed herein may further comprise a pharmaceutically or physiologically acceptable carrier, such as a dermatologically or an otically acceptable carrier. Such carriers, in the case of dermatologically acceptable carriers, may be compatible with skin, nails, mucous membranes, tissues, and/or hair, and can include any conventionally used dermatological carrier meeting these requirements. In the case of otically acceptable carriers, the carrier may be compatible with all parts of the ear. Such carriers can be readily selected by one of ordinary skill in the art. Carriers for topical administration of the compounds disclosed herein include, but are not limited to, mineral oil, liquid petroleum, white petroleum, propylene glycol, polyoxyethylene and/or polyoxypropylene compounds, emulsifying wax, sorbitan monostearate, polysorbate 60, cetyl esters wax, cetearyl alcohol, 2-octyldodecanol, benzyl alcohol, and water. In formulating skin ointments, the active components of the present disclosure may be formulated in an oleaginous hydrocarbon base, an anhydrous absorption base, a water-in-oil absorption base, an oil-in-water water-removable base, and/or a water-soluble base. In formulating otic compositions, the active components of the present disclosure may be formulated in an aqueous polymeric suspension including such carriers as dextrans, polyethylene glycols, polyvinylpyrrolidone, polysaccharide gels, Gelrite®, cellulosic polymers like hydroxypropyl methylcellulose, and carboxy-containing polymers such as polymers or copolymers of acrylic acid, as well as other polymeric demulcents. The topical compositions as disclosed herein may be in any form suitable for topical application, including aqueous, aqueous-alcoholic or oily solutions; lotion or serum dispersions; aqueous, anhydrous or oily gels; emulsions obtained by dispersion of a fatty phase in an aqueous phase (0/W or oil in water) or, conversely, dispersion of an aqueous phase in a fatty phase (W/O or water in oil), microemulsions or alternatively microcapsules, microparticles or lipid vesicle dispersions of ionic and/or nonionic type, creams, lotions, gels, foams (which may use a pressurized canister, a suitable applicator, an emulsifier, and an inert propellant), essences, milks, suspensions, or patches. Topical compositions disclosed herein may also contain adjuvants such as hydrophilic or lipophilic gelling agents, hydrophilic or lipophilic active agents, preserving agents, antioxidants, solvents, fragrances, fillers, sunscreens, odor-absorbers, and dyestuffs. In a further aspect, the topical compositions disclosed herein may be administered in conjunction with devices such as transdermal patches, dressings, pads, wraps, matrices and bandages capable of being adhered or otherwise associated with the skin or other tissue or organ of a subject, being capable of delivering a therapeutically-effective amount of one or more modified lysin polypeptides as disclosed herein.

In some embodiments, the topical compositions disclosed herein additionally comprise one or more components used to treat topical burns. Such components may include, but are not limited to, a propylene glycol hydrogel; a combination of a glycol, a cellulose derivative and a water-soluble aluminum salt; an antiseptic; an antibiotic; and a corticosteroid. Humectants (such as solid or liquid wax esters), absorption promoters (such as hydrophilic clays, or starches), viscosity building agents, and skin-protecting agents may also be added. Topical formulations may be in the form of rinses such as mouthwash. See, e.g., WO2004/004650.

The modified lysin polypeptides disclosed herein may also be administered by injection of a therapeutic agent comprising the appropriate amount of a modified lysin polypeptide and a carrier. For example, the modified lysin polypeptides can be administered intramuscularly, intracerebrovetricularly, intrathecally, subdermally, subcutaneously, intreaperitoneally, intravenously, or by direct injection or continuous infusion to treat infections by bacteria, such as gram-positive bacteria. The carrier may be comprised of distilled water, a saline solution, albumin, a serum, or any combinations thereof. Additionally, pharmaceutical compositions of parenteral injections can comprise pharmaceutically-acceptable aqueous or nonaqueous solutions of modified lysin polypeptides in addition to one or more of the following: pH buffered solutions, adjuvants (e.g., preservatives, wetting agents, emulsifying agents, stabilizing agents, and dispersing agents), liposomal formulations, nanoparticles, dispersions, suspensions, and emulsions, as well as sterile powders for reconstitution into sterile injectable solutions or dispersions just prior to use.

In certain embodiments, formulations for injection can be presented in unit dosage form, e.g., in ampoules or in multi-dose containers, and in certain embodiments may include an added preservative. The compositions can take such forms as excipients, suspensions, solutions, or emulsions in oily or aqueous vehicles, and can contain formulatory agents such as suspending, stabilizing, bulking, and/or dispersing agents. The active ingredient can be in powder form for reconstitution with a suitable vehicle, e.g., sterile pyrogen-free water, before use. Examples of buffering agents may include histidine, Tris, phosphate, succinate citrate, methionine, cystine, glycine, mild surfactants, calcium, and magnesium. A reducing agent such as dithiothreitol can also be included.

In cases where parenteral injection is the chosen mode of administration, an isotonic formulation may be used. Generally, additives for isotonicity can include sodium chloride, dextrose, sucrose, glucose, trehalose, mannitol, sorbitol, and lactose. In some cases, isotonic solutions such as phosphate buffered saline may be used. Stabilizers can include histidine, methionine, glycine, arginine, gelatin, and albumin, such as human or bovine serum albumin A person of ordinary skill will readily appreciate that many of the foregoing excipients can also be used in compositions for injection.

A vasoconstriction agent can be added to the compositions disclosed herein. In certain embodiments, the compositions may be provided sterile and pyrogen-free.

In another embodiment, the compositions disclosed herein may be dry inhalable powders or other inhalable compositions, such as aerosols or sprays. The inhalable compositions disclosed herein can further comprise a pharmaceutically acceptable carrier. For administration by inhalation, the modified lysin polypeptides may be conveniently delivered in the form of an aerosol spray presentation from such devices as inhalers, pressurized aerosol dispensers, or nebulizers, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the active ingredient and a suitable powder base such as lactose or starch.

In one embodiment, modified lysin polypeptides disclosed herein may be formulated as a dry, inhalable powder or as an aerosol or spray. In specific embodiments, modified lysin polypeptide inhalation solution may further be formulated with a propellant for aerosol delivery. In certain embodiments, solutions may be nebulized. Many dispensing devices are available in the art for delivery of pharmaceutical compositions, including polypeptides, by inhalation. These include nebulizers, pressurized aerosol dispensers, and inhalers.

A surfactant can be added to an inhalable pharmaceutical composition as disclosed herein in order to lower the surface and interfacial tension between the medicaments and the propellant. Where the medicaments, propellant, and excipient are to form a suspension, a surfactant may or may not be required. Where the medicaments, propellant, and excipient are to form a solution, a surfactant may or may not be necessary, depending in part on the solubility of the particular medicament and excipient. The surfactant may be any suitable, non-toxic compound that is non-reactive with the medicament and that reduces the surface tension between the medicament, the excipient, and the propellant and/or acts as a valve lubricant.

Examples of suitable surfactants include, but are not limited to: oleic acid; sorbitan trioleate; cetyl pyridinium chloride; soya lecithin; polyoxyethylene(20) sorbitan monolaurate; polyoxyethylene (10) stearyl ether; polyoxyethylene (2) oleyl ether; polyoxypropylene-polyoxyethylene ethylene diamine block copolymers; polyoxyethylene(20) sorbitan monostearate; polyoxyethylene(20) sorbitan monooleate; polyoxypropylene-polyoxyethylene block copolymers; castor oil ethoxylate; and combinations thereof.

Examples of suitable propellants include, but are not limited to: dichlorodifluoromethane, trichlorofluoromethane, dichloro-tetrafluoroethane, and carbon dioxide.

Examples of suitable excipients for use in inhalable compositions include, but are not limited to: lactose, starch, propylene glycol diesters of medium chain fatty acids; triglyceride esters of medium chain fatty acids, short chains, or long chains, or any combination thereof; perfluorodimethylcyclobutane; perfluorocyclobutane; polyethylene glycol; menthol; lauroglycol; diethylene glycol monoethylether; polyglycolized glycerides of medium chain fatty acids; alcohols; eucalyptus oil; short chain fatty acids; and combinations thereof.

In some embodiments, the compositions disclosed herein comprise nasal applications. Nasal applications include, for instance, nasal sprays, nasal drops, nasal ointments, nasal washes, nasal injections, nasal packings, bronchial sprays and inhalers, or indirectly through use of throat lozenges, mouthwashes or gargles, or through the use of ointments applied to the nasal nares, or the face or any combination of these and similar methods of application.

Compositions disclosed herein can also be formulated for rectal administration, e.g., as suppositories or retention enemas (e.g., containing conventional suppository bases such as cocoa butter or other glycerides).

In certain embodiments, the compositions disclosed herein may further comprise at least one antibiotic, such as at least one antibiotic effective to inhibit the growth, reduce the population, or kill at least one species of Gram-positive bacteria. In certain embodiments, the at least one antibiotic is effective against one or more of Staphylococcus aureus; Listeria monocytogenes; a coagulase negative staphylococcus such as from the Staphylococcus epidermidis group, the Staphylococcus saprophyticus group, the Staphylococcus simulans group, the Staphylococcus intermedius group, the Staphylococcus sciuri group, and the Staphylococcus hyicus group; Streptococcus suis; Streptococcus pyogenes; Streptococcus agalactiae; Streptococcus dysgalactiae; Streptococcus pneumoniae; species included in the viridans streptococci group such as the Streptococcus anginosis group, Streptococcus mitis group, Streptococcus sanguinis group, Streptococcus bovis group, Streptococcus salivarius group, and Streptococcus mutans group; Enterococcus faecalis; and Enterococcus faecium.

In certain embodiments of the compositions disclosed herein, the modified lysin polypeptide in combination with the at least one antibiotic may exhibit synergism, for example synergism in the modified lysin polypeptide's or the antibiotic's ability to inhibit the growth, reduce the population, or kill at least one species of Gram-positive bacteria. Synergy may refer to the inhibitory activity of a combination of two active agents, wherein the fractional inhibitory concentration (FIC) index for the combination is less than 1, and for strong synergy, less than or equal to 0.5. The FIC of an agent is the minimum concentration of that agent that kills bacteria when used in combination with another agent divided by the concentration of the first agent that has the same effect when the first agent is used alone. The FIC index for the combination of A and B is the sum of their individual FIC values.

Synergy may be evaluated in a checkerboard assay (and can be validated by time-kill curves). Each checkerboard assay generates many different combinations, and, by convention, the FIC values of the most effective combination are used in calculating the FIC index. The FIC index defines the nature of the interaction. Antimicrobial agents with additive interactions have a FIC index of 1; an FIC index of <1 defines synergistic interactions; combinations with an FIC index >1 are antagonistic. The lower the FIC index, the more synergistic a combination. See, e.g., Singh, P. K. et al, Am J Physiol Lung Cell Mol Physiol 279: L799-L805, 2000. Synergy has implications for an efficacious, new general anti-infective strategy based on the co-administration of modified lysin polypeptides and antibiotics. In particular each and both modified lysin polypeptides and antibiotics may be administered at reduced doses and amounts, with enhanced bactericidal and bacteriostatic activity and with reduced risk of resistance development. In other words, the benefits of synergy are not only realized when one or both agents are used at sub-MIC concentrations, although the existence of synergy may be revealed by testing with sub-MIC concentrations of each agent. Methods

Due to their high degree of activity and their low toxicity, together presenting a favorable therapeutic index, the modified lysin polypeptides disclosed herein may be administered to a subject in need thereof, e.g., a living animal (including a human) for the treatment, alleviation, or amelioration, palliation, or elimination of an indication or condition which is susceptible thereto.

Accordingly, the modified lysin polypeptides of the present disclosure can be used in vivo, for example, to treat bacterial infections due to Gram-positive bacteria, such as S. aureus, in a subject, as well as in vitro, for example to reduce the level of bacterial contamination on, for example, a surface, e.g., of a medical device.

For example, in some embodiments, the present modified lysin polypeptides may be used for the prevention, control, disruption, and treatment of bacterial biofilm formed by Gram-positive bacteria Biofilm formation occurs when microbial cells adhere to each other and are embedded in a matrix of extracellular polymeric substance (EPS) on a surface. The growth of microbes in such a protected environment that is enriched with biomacromolecules (e.g. polysaccharides, nucleic acids and proteins) and nutrients allow for enhanced microbial cross-talk and increased virulence. Biofilm may develop in any supporting environment including living and nonliving surfaces such as the mucus plugs of the CF lung, contaminated catheters, implants, contact lenses, etc (Sharma et al. Biologicals, 42(1):1-7 (2014), which is herein incorporated by reference in its entirety). Because biofilms protect the bacteria, they are often more resistant to traditional antimicrobial treatments, making them a serious health risk, which is evidenced by more than one million cases of catheter-associated urinary tract infections (CAUTI) reported each year, many of which can be attributed to biofilm-associated bacteria (Donlan, R M (2001) Emerg Infect Dis 7(2):277-281; Maki D and Tambyah P (2001) Emerg Infect Dis 7(2):342-347).

Thus, in one embodiment, the modified lysin polypeptides of the present disclosure can be used for the prevention, control, disruption, and treatment of bacterial infections due to Gram-positive bacteria when the Gram-positive bacteria are protected by a bacterial biofilm.

In one aspect, the present disclosure is directed to a method of treating a bacterial infection caused by one or more species of Gram-positive bacteria as described herein, comprising administering to a subject diagnosed with, at risk for, or exhibiting symptoms of a bacterial infection, a pharmaceutical composition as described herein.

The terms “infection” and “bacterial infection” are meant to include respiratory tract infections (RTIs), such as respiratory tract infections in patients having cystic fibrosis (CF), lower respiratory tract infections, such as acute exacerbation of chronic bronchitis (ACEB), acute sinusitis, community-acquired pneumonia (CAP), hospital-acquired pneumonia (HAP) and nosocomial respiratory tract infections; sexually transmitted diseases, such as gonococcal cervicitis and gonococcal urethritis; urinary tract infections; acute otitis media; sepsis including neonatal septisemia and catheter-related sepsis; and osteomyelitis. Infections caused by drug-resistant bacteria and multidrug-resistant bacteria are also contemplated.

Non-limiting examples of infections caused by Gram-positive bacterial may include: A) Nosocomial infections: 1. Respiratory tract infections especially in cystic fibrosis patients and mechanically-ventilated patients; 2. Bacteraemia and sepsis; 3. Wound infections, particularly those of burn victims; 4. Urinary tract infections; 5. Post-surgery infections on invasive devises; 6. Endocarditis by intravenous administration of contaminated drug solutions; 7. Infections in patients with acquired immunodeficiency syndrome, cancer chemotherapy, steroid therapy, hematological malignancies, organ transplantation, renal replacement therapy, and other conditions with severe neutropenia. B) Community-acquired infections: 1. Community-acquired respiratory tract infections; 2. Meningitis; 3. Folliculitis and infections of the ear canal caused by contaminated water; 4. Malignant otitis externa in the elderly and diabetics; 5. Osteomyelitis of the caleaneus in children; 6. Eye infections commonly associated with contaminated contact lens; 7. Skin infections such as nail infections in people whose hands are frequently exposed to water; 8. Gastrointestinal tract infections; and 9. Muscoskeletal system infections.

The one or more species of Gram-positive bacteria of the present methods may include any of the species of Gram-positive bacteria as described herein or known in the art. Typically, the species of Gram-positive bacteria may include Listeria monocytogenes, Staphylococcus aureus, coagulase negative staphylococci (including at least 40 recognized species including, but not limited to, the Staphylococcus epidermidis group, the Staphylococcus saprophyticus group, the Staphylococcus simulans group, the Staphylococcus intermedius group, the Staphylococcus sciuri group, the Staphylococcus hyicus group, and any isolates referred to as from the “unspecified species group”), Streptococcus suis, Streptococcus pyogenes, Streptococcus agalactiae, Streptococcus dysgalactiae, Streptococcus pneumoniae, any additional species included in the viridans streptococci group (including, but not limited to, all species and strains included in the Streptococcus anginosis group, Streptococcus mitis group, Streptococcus sanguinis group, Streptococcus bovis (now gallolyticus) group, Streptococcus salivarius group, and Streptococcus mutans group), Enterococcus faecalis, and Enterococcus faecium. Other examples of Gram-positive bacteria include but are not limited to the genera Actinomyces, Bacillus, Lactococcus, Mycobacterium, Corynebacterium, and Clostridium.

In another aspect, the present disclosure is directed to a method of preventing or treating a bacterial infection comprising co-administering to a subject diagnosed with, at risk for, or exhibiting symptoms of a bacterial infection, a combination of a first effective amount of the composition containing an effective amount of a modified lysin polypeptide as described herein, and a second effective amount of an antibiotic suitable for the treatment of Gram-positive bacterial infection.

The modified lysin polypeptides of the present disclosure can be co-administered with standard care antibiotics or with antibiotics of last resort, individually or in various combinations as within the skill of the art. Traditional antibiotics used against Gram-positive bacteria are described herein and may include, for example, methicillin, vancomycin, daptomycin, mupirocin, lysostaphin, penicillins, cloxacillin, erythromycin, carbapenems, cephalosporins, glycopeptides, lincosamides, azithromycin, clarithromycin, roxithromycin, telithromycin, spiramycin, and fidaxomicin.

Combining the modified lysin polypeptides of the present disclosure with antibiotics provides an efficacious antibacterial regimen. In some embodiments, co-administration of the modified lysin polypeptides of the present disclosure with one or more antibiotics may be carried out at reduced doses and amounts of either the modified lysin polypeptides or the antibiotic or both, and/or reduced frequency and/or duration of treatment with augmented bactericidal and bacteriostatic activity, reduced risk of antibiotic resistance and with reduced risk of deleterious neurological or renal side effects (such as those associated with colistin or polymyxin B use). As used herein the term “reduced dose” refers to the dose of one active ingredient in the combination compared to monotherapy with the same active ingredient. In some embodiments, the dose of the modified lysin polypeptide or the antibiotic in a combination may be suboptimal or even subthreshold compared to the respective monotherapy.

In some embodiments, the present disclosure provides a method of augmenting antibiotic activity of one or more antibiotics against Gram-positive bacteria compared to the activity of said antibiotics used alone by administering to a subject one or more modified lysin polypeptides disclosed herein together with an antibiotic of interest. The combination is effective against the bacteria and permits resistance against the antibiotic to be overcome and/or the antibiotic to be employed at lower doses, decreasing undesirable side effects.

In yet another aspect, the present disclosure is directed to a method of inhibiting the growth, or reducing the population, or killing of at least one species of Gram-positive bacteria, the method comprising contacting the bacteria with a composition containing an effective amount of modified lysin polypeptide as described herein, wherein the modified lysin polypeptide inhibits the growth, or reduces the population, or kills at least one species of Gram-positive bacteria.

In some embodiments, inhibiting the growth, or reducing the population, or killing at least one species of Gram-positive bacteria comprises contacting bacteria with the modified lysin polypeptides as described herein, wherein the bacteria are present on a surface of e.g., medical devices, floors, stairs, walls and countertops in hospitals and other health related or public use buildings and surfaces of equipment in operating rooms, emergency rooms, hospital rooms, clinics, and bathrooms and the like.

Examples of medical devices that can be protected using the modified lysin polypeptides described herein include but are not limited to tubing and other surface medical devices, such as urinary catheters, mucous extraction catheters, suction catheters, umbilical cannulae, contact lenses, intrauterine devices, intravaginal and intraintestinal devices, endotracheal tubes, bronchoscopes, dental prostheses and orthodontic devices, surgical instruments, dental instruments, tubings, dental water lines, fabrics, paper, indicator strips (e.g., paper indicator strips or plastic indicator strips), adhesives (e.g., hydrogel adhesives, hot-melt adhesives, or solvent-based adhesives), bandages, tissue dressings or healing devices and occlusive patches, and any other surface devices used in the medical field. The devices may include electrodes, external prostheses, fixation tapes, compression bandages, and monitors of various types. Medical devices can also include any device which can be placed at the insertion or implantation site such as the skin near the insertion or implantation site, and which can include at least one surface which is susceptible to colonization by Gram-positive bacteria. Dosage and Administration

Dosages administered depend on a number of factors such as the activity of infection being treated; the age, health and general physical condition of the subject to be treated; the activity of a particular modified lysin polypeptide; the nature and activity of the antibiotic if any with which a modified lysin polypeptide according to the present disclosure is being paired; and the combined effect of such pairing. In certain embodiments, effective amounts of the modified lysin polypeptide to be administered may fall within the range of about 0.1-100 mg/kg (or 1 to 100 mcg/ml), such as from 0.5 mg/kg to 30 mg/kg. In certain embodiments, the modified lysin polypeptide may be administered 1-4 times daily for a period ranging from 1 to 14 days. The antibiotic if one is also used may be administered at standard dosing regimens or in lower amounts in view of any synergism. All such dosages and regimens, however, (whether of the modified lysin polypeptide or any antibiotic administered in conjunction therewith) are subject to optimization. Optimal dosages can be determined by performing in vitro and in vivo pilot efficacy experiments as is within the skill of the art but taking the present disclosure into account.

It is contemplated that the modified lysin polypeptides disclosed herein may provide a rapid bactericidal and, when used in sub-MIC amounts, may provide a bacteriostatic effect. It is further contemplated that the modified lysin polypeptides disclosed herein may be active against a range of antibiotic-resistant bacteria. Based on the present disclosure, in a clinical setting, the present modified lysin polypeptides may be a potent alternative (or additive) for treating infections arising from drug- and multidrug-resistant bacteria alone or together with antibiotics (including antibiotics to which resistance has developed).

In some embodiments, time exposure to the modified lysin polypeptides disclosed herein may influence the desired concentration of active polypeptide units per ml. Carriers that are classified as “long” or “slow” release carriers (such as, for example, certain nasal sprays or lozenges) may possess or provide a lower concentration of polypeptide units per ml but over a longer period of time, whereas a “short” or “fast” release carrier (such as, for example, a gargle) may possess or provide a high concentration of polypeptide units (mcg) per ml but over a shorter period of time. There are circumstances where it may be desirable to have a higher unit/ml dosage or a lower unit/ml dosage.

For the modified lysin polypeptides of the present disclosure, the therapeutically effective dose may be estimated initially either in cell culture assays or in animal models, usually mice, rabbits, dogs, or pigs. The animal model can also be used to achieve a desirable concentration range and route of administration. Obtained information can then be used to determine the effective doses, as well as routes of administration, in humans. Dosage and administration can be further adjusted to provide sufficient levels of the active ingredient or to maintain the desired effect. Additional factors that may be taken into account include the severity of the disease state; age, weight and gender of the patient; diet; desired duration of treatment; method of administration; time and frequency of administration; drug combinations; reaction sensitivities; tolerance/response to therapy; and the judgment of a treating physician.

A treatment regimen can entail daily administration (e.g., once, twice, thrice, etc. daily), every other day (e.g., once, twice, thrice, etc. every other day), semi-weekly, weekly, once every two weeks, once a month, etc. In one embodiment, treatment can be given as a continuous infusion. Unit doses can be administered on multiple occasions. Intervals can also be irregular as indicated by monitoring clinical symptoms. Alternatively, the unit dose can be administered as a sustained release formulation, in which case less frequent administration may be used. Dosage and frequency may vary depending on the patient. It will be understood by one of skill in the art that such guidelines will be adjusted for localized administration, e.g., intranasal, inhalation, rectal, etc., or for systemic administration, e.g., oral, rectal (e.g., via enema), intramuscular (i.m.), intraperitoneal (i.p.), intravenous (i.v.), subcutaneous (s.c.), transurethral, and the like.

### EXAMPLES

The modified lysin polypeptides described herein and their preparation, characterization, and use will be better understood in connection with the following examples, which are intended as an illustration of and not a limitation upon the scope of the present disclosure.

The following abbreviations are used herein:

| | |
|---|---|
| CHAP | Cysteine histidine-dependent |
| | amidohydrolase/peptidase (CHAP) endopeptidase |
| | domain (the enzymatically active domain |
| | or EAD) of the lysin molecule |
| SH3b | C-terminal SH3b_5 (“SH3b”) cell wall-binding |
| | domain (or CBD) of a lysin |
| | molecule |
| MIC | Minimum inhibitory concentration, |
| | typically measured in micrograms per |
| | milliliter, indicating the minimum |
| | concentration sufficient to suppress at least 80% |
| | of the bacterial growth observed in the control |
| X#Y | Notation for mutations. The number |
| | designates the position in the sequence of |
| | wild-type PlySs2 lysin having the amino |
| | acid sequence of SEQ ID NO: 1; X is the |
| | original amino acid residue in the wild-type |
| | lysin, and Y is the replacement amino |
| | acid residue in the modified lysin polypeptide |
| VAN | Vancomycin |
| DAP | Daptomycin |
| WT | Wild type |
| TCE | T-cell epitope |

### Example 1—Selection of Modified Lysin Polypeptides

Modified lysin polypeptides disclosed herein resulted from a large screening program involving in silico, computationally-guided identification of core sequences of putative T-cell epitopes (TCE) of the wild-type PlySs2 lysin and assignment of an arbitrary TCE score to the wild-type PlySs2 lysin. In silico screening methods and algorithms were used on a commercial (fee-for-service) basis to identify potential immunogenically-relevant segments (putative T cell epitopes) of the wild-type PlySs2 lysin sequence and target these segments for mutation. Mutations were identified that were designed to disrupt the predicted TCEs. Commercially-available services can be used for assessment of the immunogenic potential of each modified lysin polypeptide. Modified lysin polypeptides can be assigned immunogenicity scores based on the disrupted TCE.

Mutation sites designed to attenuate or delete the putative TCEs were then computationally-designed, and replacement amino acids were computationally-selected to reduce the TCE score of a TCE segment. Seven amino acid positions were selected for mutation in each of the identified TCE 1, TCE 2, and TCE 3, and 47 amino acid positions were selected in the identified TCE 4. A total of 16,121 variants with mutations solely in the CHAP domain were generated. In a similar fashion, 5, 8, 19, and 21 amino acid residues were selected for potential replacement in the identified SH3b TCEs 5, 6, 7, and 8, respectively, and a total of 15,960 variants with mutations in the SH3b domain were generated. Libraries of each set of mutants (a CHAP domain and a SH3b domain library) were then screened for ability to cleave the peptidoglycan of the bacterial cell wall using the overlay test.

As shown in Table 2 below, the following segments (located respectively in the catalytic and cell wall-binding domains) of the wild type amino acid sequence of PlySs2 having the sequence of SEQ ID NO: 1, were identified as constituting core segments of putative T-cell epitopes (“TCE”) or as potentially forming part of one or more T-cell epitopes.

TABLE 2

| WT Segment | 1 | 2 | 3 | 4 | 5 | 6 | 7 | 8 |
|---|---|---|---|---|---|---|---|---|
| WT Domain | CHAP | | | | SH3b | | | |
| AA Residues | 32-45 | 84-98 | 100-112 | 128-145 | 164-170 | 172-187 | 189-201 | 204-221 |
| TCE # | 1 | 2 | 3 | 4 | 5 | 6 | 7 | 8 |
| TCE score | 4 | 4 | 7 | 10 | 4 | 3 | 4 | 6 |

Mutations were then identified that were predicted to reduce a TCE score of the resulting polypeptide by “depleting” (eliminating or weakening) one or more putative TCEs. A library encompassing up to 16,121 CHAP domain variants and 15,960 SH3b domain variants was cloned into pBAD24 expression vector, transformed into E. coli Top10 cells, and characterized in the primary screen to identify modified lysins with bacteriolytic activity. The primary screen for lytic activity was conducted using a plate-based lysogeny broth (LB) soft agar overlay method previously developed to detect lysin killing activity using lysin-expressing clones without need to purify first. However, any other method of ascertaining lytic activity can be used.

Briefly, all library members (i.e., clones) and a vector control strain were patched onto LB glass plates supplemented with ampicillin, and resulting colonies were then induced by nebulization with 0.2% arabinose vapors for 1 hour. Following induction, the plates were incubated overnight to enable expression of recombinant proteins. After expression, the library was exposed to chloroform vapors for 30 minutes to permeabilize the E. coli cell wall, air dried, and then overlaid with molten soft LB agar containing 75 μl of an overnight culture of S. aureus strain MW2 in LB. The overlaid plates were allowed to set for 15 minutes at room temperature and were then incubated for 16-24 hours at 37° C. The restrictive growth conditions for S. aureus in the overlay over clones expressing active lysin enables the appearance of distinct clearing zones that facilitate lysin identification. The vector control has no clearing zones.

In certain embodiments of the screening, the protein may be overexpressed, and in certain embodiments, the protein may not be overexpressed. Likewise, in certain embodiments, the protein may be purified, and in certain embodiments, the protein may not be purified. In one exemplary embodiment, the protein may be overexpressed and not purified.

Positive clones (i.e., clones associated with distinct clearing zones) were subcultured to ensure clonal purity and then sequenced to confirm the unique sequence and to determine the deimmunization (DI) score of the active variant. Over 1000 active variants with a range of DI scores were identified in this manner. Active clones with the lowest DI scores were expressed at high levels using the pBAD24 expression vector in E. coli Top10 cells, purified by column chromatography, and further selected based on a combination of information, including MIC values in both Mueller-Hinton broth and 100% human serum, large clearing zones on agar plates, thermal stability, and ease of purification.

Based on these criteria, 36 CHAP variants and 66 SH3b domain variants were identified. The sequence modifications of each of the 36 CHAP domains and each of the 66 SH3b domains were then combined in a single E. coli library to yield a total of up to 2376 clones combining the modifications of each domain. The combined library was then screened, as above, using the soft agar overlay method, identifying 530 “combined” mutants/variants expressing bacteriolytic activity. The mutants can be termed chimeric or shuffled as they combine a catalytic domain from one variant with a binding domain of another variant. The sequences of each clone were identified, and the putative protein sequence of each was scored for immunogenicity. Several in silico computational methods for predicting the deimmunization potential of various T-cell epitope mutations are known and are available commercially, for example from companies such as Stealth Biologics, LLC [18]. Active clones with the lowest DI scores were then chosen for overexpression, purification, further characterization, and testing for lytic activity using the MIC assay. A subset thereof was further tested for in vivo efficacy in the mouse neutropenic thigh infection model (MNTI). Based on the in vitro activity (low MIC value), in vivo efficacy using a MNTI similar to wild-type protein, high thermal stability, high purification yield, and a low TCE score (for example at least 25% lower, such as at least 40% lower than the wild type (WT) TCE score of 42, the most active modified lysin polypeptides were then selected. The amino acid sequences of the finally-selected modified lysin polypeptides (referencing differences from SEQ ID NO: 1 and the positions of its amino acid residues) are summarized in Table 3 below (using one-letter amino acid codes) as follows:

TABLE 3

| | MIC | | CHAP Domain Mutations (TCE 1-4)** | | | | SH3b Domain Mutations (TCE 5-8)** | | | |
|---|---|---|---|---|---|---|---|---|---|---|
| No. | (μg/mL) | TCE | TCE 1 | TCE 2 | TCE3 | TCE4 | TCE 5 | TCE6 | TCE 7 | TCE 8 |
| WT | 1 | 42 | DI = 4 | DI = 4 | DI = 7 | DI = 10 | DI = 4 | DI = 3 | DI = 4 | DI = 6 |
| pp55* | 0.125 | 24 | | L92W | V104S | V128T | | | | |
| | | | | | | and | | | | |
| | | | | | | Y137S | | | | |
| | | | DI = 4 | DI = 0 | DI = 2 | DI = 1 | DI = 4 | DI = 3 | DI = 4 | DI = 6 |
| pp388 | 4 | 26 | | | | | Y164N | N184D | R195E | V204K |
| | | | | | | | | | | and |
| | | | | | | | | | | V212E |
| | | | DI = 4 | DI = 4 | DI = 7 | DI = 10 | DI = 0 | DI = 0 | DI = 0 | DI = 1 |
| pp61* | 0.5 | 19 | | L92W | V104S | V128T | | | S198H | I206E |
| | | | | | | and | | | | |
| | | | | | | Y137S | | | | |
| | | | DI = 4 | DI = 0 | DI = 2 | DI = 1 | DI = 4 | DI = 3 | DI = 1 | DI = 4 |
| pp65* | 1 | 15 | | L92W | V104S | V128T | | | S198Q | V204A |
| | | | | | | and | | | | and |
| | | | | | | Y137S | | | | V212A |
| | | | DI = 4 | DI = 0 | DI = 2 | DI = 1 | DI = 4 | DI = 3 | DI = 0 | DI = 1 |
| pp296 | 2, 1 | 13 | | L92W | V104S | V128T | Y164K | N184D | S198Q | |
| | | | | | | and | | | | |
| | | | | | | Y137S | | | | |
| | | | DI = 4 | DI = 0 | DI = 2 | DI = 1 | DI = 0 | DI = 0 | DI = 0 | DI = 6 |
| pp616 | 0.5 | 29 | | | | V128T | Y164K | | | |
| | | | | | | and | | | | |
| | | | | | | Y137S | | | | |
| | | | DI = 4 | DI = 4 | DI = 7 | DI = 1 | DI = 0 | DI = 3 | DI = 4 | DI = 6 |
| pp400 | 2 | 21 | R35E | L92W | V104S | V128T | | | | |
| | | | | | | and | | | | |
| | | | | | | Y137S | | | | |
| | | | DI = 1 | DI = 0 | DI = 2 | DI = 1 | DI = 4 | DI = 3 | DI = 4 | DI = 6 |
| pp628 | 4 | 15 | | L92W | V104S | V128T | Y164K | | | V204K |
| | | | | | | and | | | | and |
| | | | | | | Y137S | | | | V212E |
| | | | DI = 4 | DI = 0 | DI = 2 | DI = 1 | DI = 0 | DI = 3 | DI = 4 | DI = 1 |
| pp632 | 8 | 8 | | L92W | V104S | V128T | Y164K | N184D | S198Q | V204K |
| | | | | | | and | | | | and |
| | | | | | | Y137S | | | | V212E |
| | | | DI = 4 | DI = 0 | DI = 2 | DI = 1 | DI = 0 | DI = 0 | DI = 0 | DI = 1 |
| pp324 | 8 | 17 | | L92W | V104S | V128T | Y164N | N184D | | |
| | | | | | | and | | | | |
| | | | | | | Y137S | | | | |
| | | | DI = 4 | DI = 0 | DI = 2 | DI = 1 | DI = 0 | DI = 0 | DI = 4 | DI = 6 |
| pp325 | 16 | 16 | | L92W | V104S | V128T | Y164N | | R195E | |
| | | | | | | and | | | | |
| | | | | | | Y137S | | | | |
| | | | DI = 4 | DI = 0 | DI = 2 | DI = 1 | DI = 0 | DI = 3 | DI = 0 | DI-6 |
| pp341 | 8 | 16 | | L92W | V104S | V128T | | N184D | | V204A |
| | | | | | | and | | | | and |
| | | | | | | Y137S | | | | V212A |
| | | | DI = 4 | DI = 0 | DI = 2 | DI = 1 | DI = 4 | DI = 0 | DI = 4 | DI = 1 |
| pp619 | 0.5 | 20 | | L92W | V104S | V128T | Y164K | | | |
| | | | | | | and | | | | |
| | | | | | | Y137S | | | | |
| | | | DI = 4 | DI = 0 | DI = 2 | DI = 1 | DI = 0 | DI = 3 | DI = 4 | DI = 6 |
| pp642 | 64 | 14 | | L92W | V104S | V128T | Y164K | | | I206E |
| | | | | | | and | | | | and |
| | | | | | | Y137S | | | | V214G |
| | | | DI = 4 | DI = 0 | DI = 2 | DI = 1 | DI = 0 | DI = 3 | DI = 4 | DI = 0 |
| pp338 | 4 | 17 | | L92W | V104S | V128T | | N184D | S198H | |
| | | | | | | and | | | | |
| | | | | | | Y137S | | | | |
| | | | DI = 4 | DI = 0 | DI = 2 | DI = 1 | DI = 4 | DI = 0 | DI = 0 | DI = 6 |

* Has extra Lysine residue at the C-terminal end, for a total of 246 amino acids.
**The DI (deimmunization) score for each of the 8 indicated TCEs in the CHAP domain and the SH3b domain is indicated.

In the foregoing table, the total DI score for each of the 8 putative TCEs of each variant is provided. MIC values are reported in the table for the variants tested. The values ranged from 0.125 μg/mL to 64 μg/mL. Wild-type lysin purified in small scale, similar to the variants, had an MIC of 1 μg/mL, while that of GMP grade had an MIC of 0.5 μg/mL. Although not wishing to be bound by theory, it is possible that the differences in wild-type activity can be attributed to purity differences.

Synergy studies with antibiotics suitable for treatment of Gram-positive bacteria, for example, daptomycin (DAP) and vancomycin (VAN), were performed using a checkerboard assay. Fractional inhibitory concentration (FIC) index values were determined for modified lysin polypeptide pp296 and antibiotic combinations against 5 methicillin-sensitive Staphylococcus aureus (MSSA) and 5 methicillin resistant Staphylococcus aureus (MRSA) isolates. An FIC index value of ≤0.5 may be interpreted as strong synergy between the two agents, while a value between 0.5 and 1 may still be considered a synergistic effect. Given that pp296 has four amino acid replacements in the catalytic domain, the ability of pp296 to synergize with antibiotics was unexpected. The present disclosure thus demonstrates the remarkable effectiveness and synergy of modified lysin polypeptides and antibiotics suitable for the treatment of Gram-positive bacteria in combination against susceptible bacteria. Qualitatively similar results may be anticipated upon performing similar assays using other de-immunized active variants, such as pp55, pp61, pp65, pp400, and pp619, in accordance with the present disclosure.

The FIC index values for modified lysin polypeptide pp296 were predominantly 0.5 with both daptomycin and vancomycin and were nearly identical to wild type, indicating that the synergistic activities of pp296 with both daptomycin and vancomycin are nearly identical to that of wild-type lysin. See Example 5 below. Qualitatively similar results may be anticipated upon performing similar assays using other de-immunized active variants, such as pp55, pp61, pp65, pp400, and pp619, in accordance with the present disclosure.

Further studies compared the anti-biofilm activity (measured as MBEC) of variant pp296 to that of wild-type PlySs2. Each was tested against 1-day-old biofilms formed by 17 MSSA and 20 MRSA isolates in a 96-well broth microdilution format. The MBEC values for modified lysin polypeptide pp296 indicate that the modified lysin polypeptide is more active against most, but not all, MSSA and MRSA isolates tested compared to wild-type PlySs2. In conclusion, the in vitro activity profile of modified lysin polypeptide pp296 may be comparable to and/or better than that of the wild-type PlySs2. See Example 6 below. These results indicate that the present modified lysin polypeptides retain or improve on the ability of the wild-type PlySs2 to disrupt, disperse, inhibit, and treat biofilms and their formation. Accordingly, it is anticipated that this activity will persist in combination with antibiotics. The present disclosure also contemplates treatment of medical devices (such as prosthetic devices; valves, such as mechanical heart valves; catheters; colostomy devices; breast implants; joint prostheses; ventricular shunts; pacemakers; defibrillators; ventricular-assisted devices; contact lenses; and contact lens cases) with the modified lysin polypeptides disclosed herein to avert bacterial attachment to surfaces of such devices that may result in formation of biofilms upon use of such devices.

Selected modified lysin polypeptides from Table 3 were subjected to further testing as described in more detail in subsequent Examples. A dose response was ascertained for several of these modified lysin polypeptides that displayed lower values of MIC (higher activity in vitro) using an in vivo MNTI model, as described below. All peptides tested in MNTI performed satisfactorily at higher doses (15 mg/kg or higher) but only pp296 performed comparably to wild type at lower doses (0.5 mg/kg). Some of the 15 modified lysin polypeptides in Table 3, in addition to wild-type controls (CF-301 and pp1149), were tested in a mouse toxicity screening to see whether they had a toxicity profile similar to the wild-type protein. Full gross necropsy of selected organs and histopathological evaluation revealed that modified lysin polypeptide pp296 caused no adventitial findings at a dose as high as 30 mg/kg. The same 30 mg/kg dose of PlySs2 caused a 100% incidence of adventitial findings, which is an indication of potential toxicity.

The results of two further toxicity studies were consistent. The first study involved an evaluation of the potential toxicity and toxicokinetic profile of pp296 when administered as a single 2-hour intravenous infusion to Sprague Dawley rats. Recovery was observed, as was persistence or progression of any effects following a minimum of a 3-day recovery period. In this study, clinical signs, body weight, body weight gain, food consumption, clinical pathology parameters (hematology, coagulation, serum chemistry, and urinalysis), gross necropsy findings, and histopathologic findings were evaluated. The study found that a single intravenous infusion of pp296 to rats at dose levels of 5, 25, 50, and 100 mg/kg was well tolerated with no adverse findings at all doses.

The second study involved prolonged and repeated exposure of animals to pp296 and evaluation of toxicity resulting from this exposure. Specifically, 0.5, 2.5, or 10 mg/kg/day were administered daily by 2-hour infusion via the tail vein for 7 consecutive days. No toxicity was found, as detailed in Example 10.

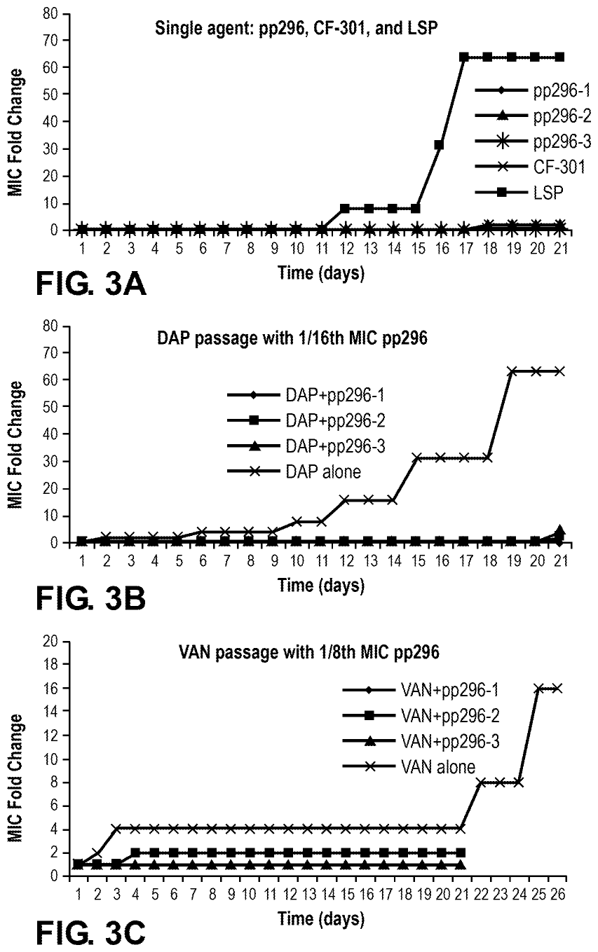

In addition, the ability of bacteria to develop resistance to the modified lysin polypeptide when exposed to modified lysin polypeptides alone and the ability of bacteria to develop resistance to either DAP or VAN when exposed to the antibiotics in the presence of sub-MIC amounts of modified lysin polypeptide was assessed. The results are shown in FIG. 3A, where no or very little increase of the MIC of modified lysin polypeptide pp296 is observed using three different replicate lineages (referred to as pp296-1, pp296-2, and pp296-3). The results are also shown in FIG. 3B and FIG. 3C, where no or very little increase in the MIC of either DAP or VAN was observed in the presence of sub-MIC amounts of modified lysin polypeptide pp296. The amount of pp296 need to suppress DAP resistance was 0.125 μg/mL, which is 1/16^{th} the MIC for pp296 alone; the suppression of DAP resistance was observed in three different replicate lineages (referred to as pp296-1, pp296-2, and pp296-3). The amount of pp296 needed to suppress development of VAN resistance was 0.25 μg/mL, which is ⅛^{th} the MIC for pp296 alone; the suppression of VAN resistance was observed in three different replicate lineages (referred to as pp296-1, pp296-2, and pp296-3). Thus, modified lysin polypeptide pp296, like the wild type lysin PlySs2, inhibits the development of antibiotic resistance and is effective in doing so even in sub-MIC amounts. For purposes of inhibiting such development, no higher amounts of lysin need be used than those resulting in sub-MIC concentrations of lysin. However, the use of higher amounts of lysin is contemplated. Based on the foregoing results, it is anticipated that pp296 will be able to overcome antibiotic resistance when administered in combination with antibiotics to which a bacterium has developed resistance.

### Characteristics of Modified Lysin Polypeptides Disclosed Herein

The modified lysin polypeptides described herein have certain similarities in that the amino acid residues replaced in the catalytic domain are for the most part concentrated in TCE 2, TCE 3, and TCE 4, with only one of the final candidates also having a substitution at TCE 1. Furthermore, the residues replaced are even more restricted, with all but two final candidates having the modifications L92W in TCE 2 and V104S in TCE 3, and all but one final candidate also have each of the substitutions V128T and Y137S. Moreover, it appears that these four substitutions in TCE 2, 3, and 4 are sufficient (without any or with minimal substitutions in the binding domain to substantially decrease immunogenicity without substantially affecting (and indeed sometimes increasing) activity. See, for example, pp55, pp400, pp619, and pp388. Thus, it is not necessary to have variation from wild type in both domains. An additional substitution in the catalytic domain, R35E, yielded a polypeptide (pp400) with both good activity and substantially (over 50%) reduced immunogenicity. It is further noteworthy that the modified lysin polypeptide pp400 had no substitutions in the binding domain. In general, however, mutations in the catalytic domain alone may not be sufficient to adequately deimmunize the lysin.

In terms of TCE score, the putative T cell epitopes of the wild type lysin have the following TCE scores, which makes the CHAP domain a more important overall contributor to immunogenicity than the SH3b domain: TCE1=4; TCE2=4, TCE3=7; TCE 4=10; TCES=4; TCE6=3; TCE7=4; and TCE8=6.

In general, the catalytic domain tolerated a more restricted variety of substitutions.

Turning to the binding domain, it was again observed that substitutions in both domains were not necessary. See, for example, modified lysin polypeptide pp388. Consistent with the observation that the catalytic domain may be a more important contributor to immunogenicity, modified lysin polypeptide pp388, which has no CHAP domain substitutions, exhibited a lower reduction in immunogenicity than active modified lysin polypeptides comprising substitutions in the CHAP domain.

The substitutions in TCEs 5, 6, 7, and 8 of the SH3b domain are restricted as follows: Y164N or Y164K (collectively, Y164N/K) in TCE 5; N184D in TCE 6; R195E, S198H or S198Q (S198H/Q) in TCE 7; and the combination V204A/K and V212A/E, or I206E alone or in combination with V214G in TCE 8.

In summary, it has been demonstrated that it is possible to create variants of the lysin PlySs2 by replacing one or more amino acid residues in one or more of positions 35, 92, 104, 128, or 137 of the CHAP domain and/or one or more amino acid residues in positions 164, 184, 195, 198, 204, 206, 212, or 214 of the SH3b domain (the positions being numbered as per the sequence of the wild-type lysin having the amino acid sequence of SEQ ID NO: 1) so as to reduce the immunogenicity and/or toxicity of the modified lysin polypeptide compared to that of wild-type lysin while maintaining substantial lytic activity against one or more target microorganisms of the wild-type lysin Immunogenicity can be measured by TCE score, using any available in silico computationally guided method for obtaining such score (for example those commercially available on a fee-for-service basis) and compared to the similarly derived TCE score of the parent lysin. For example, Stealth Biologics, ProImmune, Creative Biolabs, Epivax and other companies may undertake such studies, and several among them may follow them up with in vitro and in vivo testing. Alternatively, immunogenicity can be assessed by any one of a number of in vitro or in vivo immunoassays, such as mixed lymphocyte reaction or PBMC proliferation assay (and assessment of proliferation, for example by detection and quantification of one or more pro-inflammatory cytokines secreted in response to stimulation with the polypeptide being tested). Ultimately, immunogenicity may be assessed against the human immune system. A number of publicly available and regulatory authority sanctioned strategies exist for doing so [6].

As can be seen from Table 3, immunogenicity may only relate in part to numbers. Generally speaking, as additional T-cell epitopes are depleted or attenuated, immunogenicity may decrease, provided that one stops short of substantially compromising lytic activity (by testing the deimmunized variants for activity, using any available test, such as the MIC assay described above). However, it is possible to strategize within the parameters and guidance given, such that a smaller number of amino acid substitutions can produce a desirable effect, as shown, for example in pp55, pp616, and pp388. Based on the foregoing data, it is theorized that an optimal number of substitutions in the CHAP domain may be three in the TCE 2, TCE 3, and TCE 4 positions, for example. However, the modified lysin polypeptides can also accommodate more than three substitutions in the CHAP domain.

### Example 2: Expression of Modified Lysin Polypeptides

The wild-type PlySs2 can be obtained as described, for example, in U.S. Pat. No. 9,034,322 to Fischetti et al. A similar procedure (plasmid pBAD24 inducible with arabinose) was followed to express the modified lysin polypeptides of the present disclosure from a library of mutant polynucleotides. A wild-type PlySs2 lysin sample was also purified by the method employed to purify the modified lysin polypeptides, and the purified wild-type PlySs2 was used as an additional positive control. ThermoFisher (InvitroGen) generated libraries of all possible CHAP domain and SH3b variants. Modified lysin polypeptides having the sequences based on SEQ ID NO: 1 with the modifications identified in Table 3 can be generated, for example, by site-directed mutagenesis from the wild-type PlySs2 lysin.

The modified lysin polypeptides were cloned into a pBAD24 vector and transformed in Escherichia coli Top 10 cells. pBAD24 encodes a β-lactamase (encoding ampicillin resistance) and enables tight control of arabinose-induced transcription. The recombinant E. coli strains were grown on LB plates, induced with arabinose vapors, and overlaid with soft agar overlays containing Staphylococcus aureus strain MW2 (Schuch, R. et al., 2009, Methods Mol Biol. 2009; 502:. doi:10.1007/978-1-60327-565-1_18). If the modified lysin polypeptides were active, clearing zones appeared around the E. coli colonies (plate-based LB soft agar overlay method).

Production was scaled up as follows: Transformed E. coli containing the pBAD24_Variant plasmid from −80° C. stock were freshly streaked, inoculated into LB media containing 100 μg/mL carbencillin and 0.2% glucose, and grown overnight. The next day, a 1 in 50 dilution of overnight culture that was spun down and re-suspended in PBS was inoculated into two-liters of LB media with 100 μg/mL carbencillin The culture was grown for 3 hours at 37° C. before the addition of arabinose to a final concentration of 0.2% and an overnight induction at 25° C. for 16+/−3 hours. The culture was aseptically centrifuged at 4000 rpm for 20 minutes. The pellets were suspended in Tris buffer pH 7.2 (cell suspension) to a volume of 30 mL, and 5 μl of 250 U/μl benzonase (total 1250 U) and one complete EDTA-free tablet of protease inhibitor (Roche) were added. The mixture was then sonicated and clarified by centrifugation at 18,000 rpm for 30 minutes. The resulting supernatant was then diluted 1:3 with 1 part of dH₂O and 1 part of 25 mM Tris buffer pH 7.2 (total volume 90-100 mL), and passed through a 0.22 micron membrane before starting the purification process.

### Example 3: Purification of Modified Lysin Polypeptides

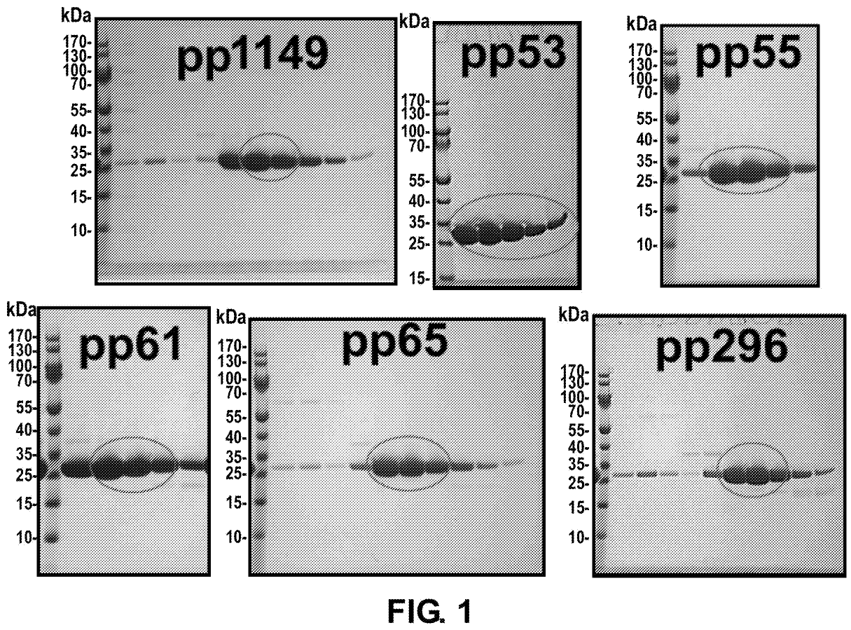

Purification of the wild-type PlySs2 and modified lysin polypeptides was performed by ion exchange chromatography followed by final size exclusion chromatography. Briefly, the resuspended cell lysate was first passed through a 5 mL Hi Trap DEAE column (weak anion exchange), then eluted with a salt gradient from a 5 mL Hi Trap Capto MMC column (weak cation exchange column with additional hydrophobic and H-bond interactions). The fractions were quantitated by OD280 and their purity assessed by SDS-PAGE gel electrophoresis. Fractions containing protein were pooled and dialyzed against 25 mM Tris buffer pH 7.4. After dialysis, the protein fractions were loaded onto a 5 mL Hi Trap Q FF column (strong anion exchange column), and the effluent was collected and loaded onto a second Capto MMC column and eluted by a salt gradient. The eluants containing protein were pooled and dialyzed against phosphate saline buffer and further purified by gel filtration (size exclusion) using a HiLoad 16/600 Superdex 75 pg column. All purified samples were stored in Demo Buffer (7.67 mM sodium phosphate monobasic dihydrate, 7.33 mM sodium phosphate dibasic monohydrate, 150 mM NaCl, pH 7.2. SDS-PAGE gels of the fractions under the elution peak of the size exclusion chromatography (last step of the purification process) are shown in FIG. 1 for some of the modified lysin polypeptides purified, including pp1149, pp53, pp55, pp61, pp65, and pp296 (and not for the wild-type PlySs2 obtained from a contract manufacturer which had already been purified).

Fractions of the modified lysin polypeptides, which are grouped in the circles as shown in FIG. 1, were pooled together.

Purification yields ranged between 4 and 94 grams of protein per 2 L batch of culture. The purified modified lysin polypeptides and wild type controls had the following characteristics as shown in Table 4 below:

TABLE 4

| Wild-type | | | Added C- | | |
|---|---|---|---|---|---|
| or Variant | CHAP | SH3b | terminal | | Predicted |
| No. | mutations | mutations | residue | MW | pI |
| WT | None | none | | 25,928 | 8.97 |
| pp1149 | None | none | | 25,928 | 8.97 |
| pp53 | None | none | +246K | 26,057 | 9.10 |
| pp55 | L92W | none | +246K | 26,059 | 8.97 |
| | V104S | | | | |
| | V128T | | | | |
| | Y137S | | | | |
| pp61 | L92W | S198H | +246K | 26,240 | 9.00 |
| | V104S | I206E | | | |
| | V128T | | | | |
| | Y137S | | | | |
| pp65 | L92W | S198Q | +246K | 26,160 | 9.13 |
| | V104S | V204A | | | |
| | V128T | V212A | | | |
| | Y137S | | | | |
| pp296 | L92W | Y164K | | 26,054 | 9.04 |
| | V104S | N184D | | | |
| | V128T | S198Q | | | |
| | Y137S | | | | |
| pp388 | | Y164N | | 25,912 | 8.32 |
| | | N184D | | | |
| | | R195E | | | |
| | | V204K | | | |
| | | V212E | | | |
| pp616 | V128T | Y164K | | 26,060 | 9.17 |
| | Y137S | | | | |
| pp400 | R35E | | | 26,019 | 8.64 |
| | L92W | | | | |
| | V104S | | | | |
| | V128T | | | | |
| | Y137S | | | | |
| pp628 | L92W | Y164K | | 26,071 | 9.17 |
| | V104S | V204K | | | |
| | V128T | V212E | | | |
| | Y137S | | | | |
| pp632 | L92W | Y164K | | 26,113 | 9.04 |
| | V104S | N184D | | | |
| | V128T | S198Q | | | |
| | Y137S | V204K | | | |
| | | V212E | | | |
| pp324 | L92W | Y164N | | 25,998 | 8.88 |
| | V104S | N184D | | | |
| | V128T | | | | |
| | Y137S | | | | |
| pp325 | L92W | Y164N | | 25,970 | 8.67 |
| | V104S | R195E | | | |
| | V128T | | | | |
| | Y137S | | | | |
| pp341 | L92W | N184D | | 25,991 | 8.85 |
| | V104S | V204A | | | |
| | V128T | V212A | | | |
| | Y137S | | | | |
| pp619 | L92W | Y164K | | 26,121 | 9.17 |
| | V104S | | | | |
| | V128T | | | | |
| | Y137S | | | | |
| pp642 | L92W | Y164K | | 25,985 | 9.04 |
| | V104S | I206E | | | |
| | V128T | V214G | | | |
| | Y137S | | | | |
| pp338 | L92W | N184D | | 26,097 | 8.85 |
| | V104S | S198H | | | |
| | V128T | | | | |
| | Y137S | | | | |

### Example 4: Thermal Stability of Modified Lysin Polypeptides

Thermal stability was assessed by incubating the lysin polypeptides at a fixed concentration of 128 μg/mL in Tris buffer pH 8.0 for 30 minutes at various elevated temperatures (within the range of about 30° C. to 60° C.) prior to assessing activity. The samples were then cooled for 2 minutes on ice and activity was then assessed by an in vitro lytic assay by exposing a bacterial inoculum (OD₆₀₀ ˜0.5-1.2) in Tris buffer to a 2-fold dilution series of each sample (diluted from 128-0.25 μg/mL across the x-axis of a 96 well microtiter plate). The loss of optical density was followed for 15 minute at room temperature, and the specific activity was determined based on the enzyme concentration enabling a 50% loss of optical density at 15 minutes. The enzyme activities of each variant were compared to the wild-type, CF-301 enzyme (GMT-grade PlySs2 protein) to calculate the % of Wild-Type Activity over a range of temperatures.

The thermal stability of wild-type, CF-301 was arbitrarily set at 100. The results for exemplary modified lysin polypeptides are set forth below in Table 5 at a single temperature of 37° C.:

TABLE 5

| Wild-type or | % Wild-Type |
|---|---|
| Variant No. | Activity at 37° C. |
| WT CF-301 | 100 |
| WT pp1149 | 95.6 |
| pp53 | 149.4 |
| pp55 | 122, 146 |
| pp61 | 258 |
| pp65 | 220 |
| pp296 | 205 |

In another experiment, the thermal stability of modified lysin polypeptides was assessed at different temperatures and compared with that of wild-type, CF-301 (set at 100). For several of the modified lysin polypeptides, including pp296, the stability was substantially higher than that of wild-type lysin at temperatures at and below 42° C. and substantially lower at temperatures at and above 45° C., as shown in Table 6, below.

TABLE 6

| | VALUES ARE REPORTED | | | | | |
|---|---|---|---|---|---|---|
| | AS A % OF CF-301 WILD-TYPE | | | | | |
| WT/Variant | 37° C. | 40° C. | 42° C. | 45° C. | 48° C. | 50° C. |
| WT pp1149 | 95.6 | 83.2 | 94.2 | 87.1 | 79.4 | 36.8 |
| pp55 | 122.1 | 110.8 | 116.7 | 82.0 | 36.5 | 11.2 |
| pp61 | 258.3 | 149.1 | 189.4 | 80.0 | 0.0 | 0.0 |
| pp65 | 219.9 | 248.4 | 270.4 | 82.1 | 0.0 | 0.0 |
| pp296 | 204.6 | 243.1 | 141.2 | 40.1 | 0.0 | 0.0 |

### Example 5: Lytic Activity of Modified Lysin Polypeptides In Vitro, Synergy with Antibiotics, and Nondevelopment of Resistance

In vitro activity of all PlySs2 variants was assessed against S. aureus isolate CFS-860 (LAIRD-426) in 100% Human Serum (HuS) and compared to wild-type PlySs2 (CF-301) and PlySs2 purified in the laboratory (referred to as pp1149). All variants were tested according to the CLSI (Clinical and Laboratory Standards Institute) broth microdilution method for assessing MIC, as follows.

Methicillin-resistant S. aureus (MRSA strain MW2) were cultured from 80° C. bacterial stocks, by plating streak isolates on BBL™ Trypticase™ soy agar II with 5% sheep blood plates for 18-24 hours at 37° C. to obtain single colonies. Colonies selected for uniform appearance were inoculated into 2.5 mL Mueller Hinton Broth (MHB). Turbidity was adjusted to 0.5 McFarland Standard Units (5×10⁵ colony-forming units [CFU]/mL) at room temperature. Standardized cultures were diluted 1:150 in 100% human serum before exposure to 2-fold serial dilutions of wild-type PlySs2 or modified lysin polypeptides in 96-well round-bottom, polystyrene microtiter plates (BD). Plates were incubated for 16 hours at 37° C. before determination of the minimal lysin concentration required to inhibit growth.

In vitro activity of the modified lysin polypeptide pp296 was assessed against a range of 51 MSSA and 51 MRSA isolates in cation-adjusted Mueller Hinton broth supplemented with 25% horse serum and 0.5 mM DTT (CAMHB-HSD) and compared to WT GMP grade PlySs2 (CF-301). CF-301 and pp296 were each diluted into CAMHB-HSD. For each lysin, the dilution series is set up such that, after a two-fold dilution of each well of the plate, the final concentrations contained: 64, 32, 16, 8, 4, 2, 1, 0.5, 0.25, and 0.125 μg/mL. Each lysin was tested in duplicate against each strain. Preparations were incubated overnight. Growth was visualized as a growth button approximately 2 mm in diameter. The highest dilution that yielded ≥80% growth inhibition was called the minimum inhibitory concentration (MIC). The activity of pp296 was very similar, within one 2-fold dilution, to that of CF-301, as shown below in Table 7.

TABLE 7

| | | pp296 (μg/mL) | | | CF-301 (μg/mL) | | |
|---|---|---|---|---|---|---|---|
| Strains | N | MIC₅₀ | MIC₉₀ | Range | MIC₅₀ | MIC₉₀ | Range |
| MSSA | 51 | 1 | 1 | 0.5-2 | 0.5 | 1 | 0.5-1 |
| MRSA | 51 | 1 | 1 | 0.25-2 | 0.5 | 0.5 | 0.12-1 |

In another experiment, the ability of modified lysin polypeptide pp296 to synergize with daptomycin and vancomycin was assessed using a checkerboard analysis. FIC (fractional inhibitory concentration) index values were determined for pp296 and antibiotic combinations against 5 MSSA and 5 MRSA isolates. A total of 50 μl of Mueller-Hinton broth (BBL) was distributed into each well of microdilution plates. The first antibiotic of the combination was serially diluted along the ordinate, while the second drug was diluted along the abscissa. An inoculum equal to a 0.5 McFarland turbidity standard was prepared from each S. aureus isolate in MHB. Each microtiter well was inoculated with 100 μl of a bacterial inoculum of 5×10⁵ CFU/ml, and the plates were incubated at 37° C. for 18 h under aerobic conditions. The resulting checkerboard contained each combination of two antibiotics, with wells that contained the highest concentration of each antibiotic at opposite corners. According to the CLSI guidelines for broth microdilution, the MIC was defined as the lowest concentration of antibiotic that completely inhibited the growth of the organism as detected with the naked eye. To quantify the interactions between the antibiotics being tested, the FIC index (the combination of antibiotics that produced the greatest change from the individual antibiotic's MIC) value was calculated for each strain and antibiotic combination as follows:

(A/MIC_{A})+(B/MIC_{B})=FIC_{A}+FIC_{B}=FIC index,

where A and B are the MIC of each agent in combination in a single well; MIC_{A} and MIC_{B} are the MIC of each drug when used individually) The combination is considered strongly synergistic when the FIC index is <0.5 and synergistic when the FIC index is >0.5-<1.

Checkerboards were generated using combinations of pp296 with either daptomycin or vancomycin against 5 MRSA strains (MW2, JMI-5675, JMI-4408, JMI-6181, and JMI-6182) and 5 MSSA strains (ATCC 29213, JMI-40979, JMI-43257, JMI-41293, and JMI-49315). Synergy was defined as inhibitory activity greater than what would be predicted by adding the two components together. With vancomycin, the modified lysin polypeptide pp296 and CF-301 exhibited identical FIC index values of 0.5 against 6 of the 10 strains examined, evincing strong synergy. pp296 exhibited a slightly superior FIC index value of 0.375 compared to that of CF-301 (FIC index value of 0.5) against one MRSA (JMI-6182) and one MSSA strain (JMI-40979). pp296 also exhibited a slightly higher FIC index value of 0.625 and 0.75 against one MSSA (ATCC 29213) and one MRSA (JMI-41293) strain compared to values of 0.5 for CF-301 against both strains. These findings indicate that pp296 exhibits levels of synergy with daptomycin and vancomycin very similar to that of CF-301.

In another experiment, it was determined that the bacteria (MRSA strain MW2) did not develop resistance to antibiotics daptomycin and vancomycin after 21-26 days of serial passage in the presence of antibiotics combined with sub-MIC amounts of pp296 at sub-MIC amounts, as did bacteria serially passaged in the presence of antibiotics without pp296.

An analysis of bacterial resistance was performed using MRSA strain MW2 over 18 days of serial passage in the presence of a pp296 dilution series and an antibiotic dilution series in presence and absence of sub-MIC amounts of pp296. Briefly, the broth microdilution MIC format was used in which 2-fold dilutions ranges of pp296 alone or antibiotic in the presence and absence of a fixed sub-MIC amount of pp296 was cultured with bacteria (5×10⁵ CFU/ml starting concentration) in MHB for 20 hours at 37° C. The well with the highest concentration of the modified lysin polypeptide in which bacterial growth was seen was then used as the inoculum for the next day's passage, and the process was repeated over a 21-26 day period. The MIC at each daily time-point was recorded, and resistance was measured as a step-wise increase in MIC. In the assay, no resistance to pp296 was observed. Moreover, sub-MIC amounts of pp296 suppressed the appearance of daptomycin and vancomycin resistance. The results are depicted graphically in FIGS. 3A-3C.

### Example 6: Anti-Biofilm Activity of Modified Lysin Polypeptides

The anti-biofilm activity of pp296 was determined against 1-day old biofilms formed by 17 MSSA and 20 MRSA isolates in a 96-well broth microdilution format. The Minimum Biofilm Eradication Concentration (MBEC) values for pp296, compared to wild-type PlySs2 (CF-301), were determined by the following standard methodology. Bacteria were suspended in PBS (0.5 McFarland Units), diluted 1:100 in TSBg (66% tryptic soy broth, 0.2% glucose), added as 0.15 mL aliquots to 96-well polystyrene microplates, and incubated for 24 hours at 37° C. Biofilms were then washed and treated with a 2-fold dilution series of CF-301 or pp296 in TSBg at 37° C. for 24 hours. All samples were examined in triplicate. After treatment, wells were washed, air-dried at 37° C., and stained with 0.05% crystal violet for 10 min The loss of biofilm biomass was assessed visually, and crystal violet was quantitated by solubilization in 33% (vol/vol) acetic acid. The optical density of 600 nm (OD₆₀₀) was determined using a SpectraMax M3 Multimode microplate reader. The MBEC of each sample was the minimum lysin concentration required to remove >95% of the biofilm biomass assessed by crystal violet quantitation. Using the MBEC assay, it was determined that pp296 was more active against most, but not all, MSSA and MRSA isolates tested compared to wild-type PlySs2. Therefore, pp296 anti-biofilm activity is overall slightly better than that of wild-type lysin, as shown in Table 8 below.

TABLE 8
Anti-biofilm activity of pp296 compared to CF-301

| | Variant 296 (μg/mL) | | | | WT PlySs2 (μg/mL) | | | |
|---|---|---|---|---|---|---|---|---|
| | N | MBEC₅₀ | MBEC₉₀ | Range | N | MBEC₅₀ | MBEC₉₀ | Range |
| MSSA | 17 | 0.063 | 0.25 | 0.01-1 | 19 | 0.125 | 0.125 | 0.125-0.5 |
| MRSA | 20 | 0.031 | 0.06 | 0.01-0.25 | 20 | 0.125 | 0.125 | 0.125-0.25 |

### Example 7: Efficacy of Modified Lysin Polypeptides In Vivo

The efficacy of four modified lysin polypeptides was compared to wild-type PlySs2 in a dose response (0-60 mg/kg) assay using a mouse neutropenic thigh infection (MNTI) model.

Bacterial inoculum was generated by allowing bacterial cells to grow to exponential phase in MHB until they reached an optical density (OD) of about 0.5 at 600 nm. The bacterial cells were thoroughly washed, resuspended in 0.9% sodium chloride USP, and diluted to an equivalent of 3.9×10⁸ colony forming units (CFU)/mL. Cells were further diluted as described below in 0.9% sodium chloride USP and maintained on wet ice.

Neutropenia was induced in female BALB/cByJ mice (5-7 weeks, Jackson Laboratories) by intraperitoneal (ip) administration of 150 mg/kg and 100 mg/kg of cyclophosphamide 4 and 1 days prior to inoculation of bacteria, respectively. Bacterial inoculum (S. aureus isolate CFS-860, 100 μl of 10⁷ CFU/ml solution) was injected intramuscularly (IM) into both posterior thighs of each mouse. Mice were dosed intravenously (0, 5, 15, 30, 60 mg/kg) with wild-type PlySs2 lysin and modified lysin polypeptides pp1149, pp55, pp61, pp65, and pp296 (2 mice per dose) beginning 2 hours post-ip infection. At 2 hours post-inoculation (Early Control) and 24 hours post-treatment initiation, animals were euthanized (CO2 asphyxiation), and the thighs were aseptically removed. Each thigh was weighed, placed into 4 ml sterile saline and homogenized in 7 ml lysing tubes using a Precellys24 high-throughput tissue homogenizer (Bertin Corp, Rockville, Md.). Dilutions of homogenate were plated on tryptic soy blood agar plates (Becton Dickinson) and incubated at 37° C. overnight in a CO₂ incubator. Bacterial burden was enumerated and expressed as log 10 CFU/g of thigh weight and compared to control animals harvested at 2 hours post infection (Early Control) and animals treated with vehicle (Late Control).

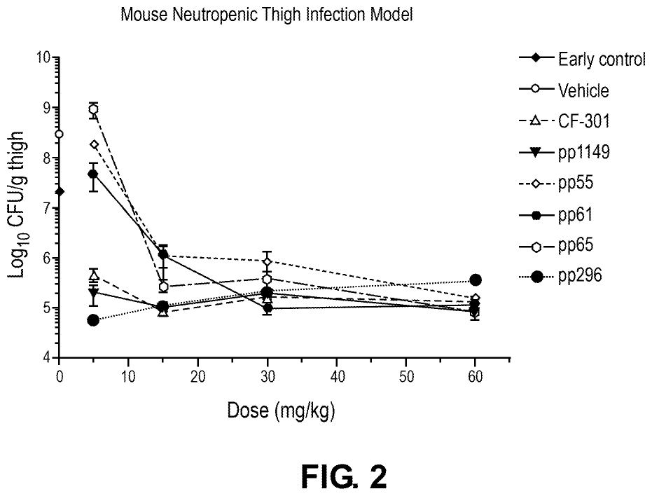

The data representing mouse thigh bacterial burden are depicted in FIG. 2. The upper dot on the y-axis is vehicle alone, and the lower dot on the y-axis is the Early Control. pp1149 is in fact also a wild-type lysin over-expressed and purified in the laboratory along with the other modified lysin polypeptides and used as an additional positive control.

It can be seen in FIG. 2 that four variants (pp55, pp61, pp65, and pp296) have activity comparable to wild-type PlySs2 lysin and that pp296 is superior, as its activity is comparable to that of wild-type PlySs2 lysin even at the lowest dose used (5 mg/kg).

### Example 8: Toxicity Screening in Mice

Mouse toxicology screenings showed that mice dosed with 30 mg/kg wild-type PlySs2 developed perivascular infiltration of mixed inflammatory cell population accompanied by adventitial lesions (or adventitial findings) in the abdominal and thoracic aorta and aortic root at the base of the heart. Adventitial findings were observed in all mice dosed with 30 mg/kg wild-type PlySs2 and wild-type PlySs2 (pp1149). Adventitial lesions were observed in 3 of 4 mice treated with a dose of 30 mg/kg of pp53, which differs from PlySs2 wild-type lysin only by an extra lysine amino acid residue at the C-terminal position 246. Mice administered a dose of ≥30 mg/kg of the remaining modified lysin polypeptides did not have any adventitial findings.

The four modified lysin polypeptides that did not result in adventitial lesions in this experiment (pp55, pp61, pp65 and pp296) had the same mutations in the catalytic domain (L92W, V104S, V128T and Y137S), suggesting that the corresponding positions in the wild-type lysin may contribute to the observed adverse effects. The presence of adventitial lesions in mice dosed with pp53 indicated that an extra lysine residue at the C-terminal end of PlySs2 lysin is not sufficient to prevent toxicity.

Other mild pathology findings were occasionally present in mice of different groups but could not be attributed to lysin administration. Minimal changes in the kidneys were observed in mice from different dose groups, and, while not wishing to be bound by theory, it is possible this difference is related to the purity of the test polypeptides since wild-type PlySs2 (CF-301) did not result in kidney findings, whereas renal changes were noted in 1 of 4 mice administered wild-type PlySs2 (pp1149). In any event, incidence of pathology was mild, not consistent, and not of concern.

Based on these experiments, pp296 appears to have the most desirable combination of features, including in vivo and in vitro activity comparable to that of the wild-type lysin, with reduced immunogenicity and reduced toxicity compared to wild-type lysin as measured by incidence of adventitial findings.

### Prophetic Example 9: In Vitro Immunogenicity Assessment

Peripheral blood mononuclear cells (PBMCs) from different donors are HLA-typed and evaluated to confirm broad representation across the global human population. PBMCs will be cultured with protein or control or without exposure to variants. PBMC samples cultured for 14 days with media exchanges and cytokine support on days 4, 7, and 11. On day 14, PBMC samples from each culture will be harvested and aliquoted into a FluoroSpot® plate pre-coated with anti-human antibodies against the cytokine of interest. Each sample is stimulated with a variant or wild-type lysin as a positive control (as either a challenge or first exposure) or no protein as a negative control prior to plate incubation.

After 24 hours, the FluoroSpot® plates will be developed with the addition of FITC-labeled anti-cytokine antibodies (for example anti-IFN-gamma antibodies). The addition of anti-FITC-490 antibody will allow spots to emerge on the membrane, which will be counted on a Zeiss automated FluoroSpot® reader system.

Positive responses (Student's T-test) will be determined based on a difference in spot counts between the exposed and unexposed samples of each variant or control. Responses across different variants will be evaluated for statistical significance (p<0.05).

### Example 10: Toxicity Study in Rats

The objectives of this study were to evaluate the potential toxicity and toxicokinetic profile of pp296 when administered as a single 2-hour intravenous infusion to Sprague Dawley rats, as well as to evaluate the recovery, persistence, or progression of any effects following a minimum of a 3-day recovery period.

The following Tables 9 and 10 present the study group arrangement. There were 5 male and 5 female rats per group in Phase A.

TABLE 9
Study Design - Phase A

| | | Dose | | Dose |
|---|---|---|---|---|
| Group | | Level | Concentration | Volume |
| Number | Treatment | (mg/kg) | (mg/mL) | (mL/kg/hr) |
| 1 | Vehicle^{a} | 0 | 0 | 5 |
| 2 | pp296 | 5 | 0.5 | 5 |
| 3 | pp296 | 25 | 2.5 | 5 |
| 4 | pp296 | 50 | 5.0 | 5 |
| 5 | pp296 | 100 | 10 | 5 |

TABLE 10
Study Design - Phase B Toxicokinetic Groups

| Group | | Dose Level | Concentration | Dose Volume | Number of Animals^{b} | |
|---|---|---|---|---|---|---|
| Number | Treatment | (mg/kg) | (mg/mL) | (mL/kg/hr) | Males/Females | |
| 6 | pp296 | 5 | 0.5 | 5 | 6 | 6 |
| 7 | pp296 | 25 | 2.5 | 5 | 6 | 6 |
| 8 | pp296 | 100 | 10 | 5 | 6 | 6 |

^{b}Phase B toxicokinelic Groups 6-8 were dosed in the same fashion as for Phase A Groups 1-5 (2-hour intravenous infusion at the dose volume specified above). There were 6 male and 6 female rats per group.

For the main (Phase A) and toxicokinetic (Phase B) phases, animals received a single 2-hour intravenous infusion. Phase A animals received a single 2-hour intravenous infusion at escalating doses until the maximum tolerated dose (MTD) was determined up to a high dose of 100 mg/kg. A minimum of 3 days of observation was allowed between each pp296 dose. Animals assigned to Groups 2 through 5 were necropsied approximately 72 hours following the end of dose administration. ^{a}The vehicle was 20 mM L-histidine, 5% D-sorbitol (w/v), pH 7.0 (±0.1).

The following parameters and endpoints were evaluated in this study: clinical signs, body weight, body weight gain, food consumption, clinical pathology parameters (hematology, coagulation, serum chemistry, and urinalysis), gross necropsy findings, and histopathologic examinations.

Based on the results of this study, a single intravenous infusion of pp296 to Crl:CD(SD) rats at dose levels of 5, 25, 50, and 100 mg/kg was well-tolerated with no adverse findings at all doses. Therefore, the no-observed-adverse-effect level (NOAEL) was considered to be >100 mg/kg, as no toxicity was observed at the highest tested dose (100 mg/kg).

### Example 11: Additional Toxicity Study in Rats

The objective of this study was to evaluate the potential toxicity and toxicokinetic profile of modified lysin polypeptide pp296 when administered daily by 2-hour infusion to Sprague Dawley rats for 7 consecutive days.

Experimental procedures, animals and treatment are summarized below in Table 11.

TABLE 11

| Group | | DoseLevel | Dose Volume^{a} | Concentration | DoseRate | No. of Animals |
|---|---|---|---|---|---|---|
| No. | Treatment | (mg/kg/day) | (mL/kg) | (mg/mL) | (mL/kg/hr) | Male/Female |
| 1 | Vehicle | 0 | 10 | 0 | 5 | 5/5 |
| 2 | pp296 | 0.5 | 0.5 | 1 | 0.25 | 5/5 |
| 3 | pp296 | 2.5 | 2.5 | 1 | 1.25 | 5/5 |
| 4 | pp296 | 10 | 10 | 1 | 5 | 5/5 |

All surviving animals were submitted for necropsy on Day 8 (Terminal Euthanasia). Necropsies were performed and organ weights were collected by Testing Facility personnel. Statistical analysis of organ weight data was performed by the testing facility engaged on a fee-for-service commercial basis. Tissues required for microscopic evaluation were trimmed, processed routinely, embedded in paraffin, and stained with hematoxylin and eosin by Charles River Laboratories Ashland, LLC. Microscopic evaluation was conducted by a board-certified veterinary pathologist on select protocol-specified tissues from all animals in Groups 1 through 4 and all gross lesions from all animals. Tissues were evaluated by light microscopy.

Sprague Dawley rats tolerated the modified lysin polypeptide pp296 at doses of 0.5, 2.5, or 10 mg/kg/day when administered daily by 2-hour infusion via the tail vein for 7 consecutive days. There were no unscheduled deaths throughout the study and no pp296-related gross, microscopic, or organ weight findings.

The present invention is not to be limited in scope by the specific embodiments described herein. Indeed, various modifications of the invention in addition to those described herein will become apparent to those skilled in the art from the foregoing description.

All patents, applications, publications, test methods, literature, and other materials cited herein are hereby incorporated by reference.

### REFERENCES

(1) Fischetti V A, Nelson D, Schuch R. Reinventing phage therapy: are the parts greater than the sum? Nat Biotechnol. 2006; 24:1508-1511.
(2) Louie L, Kaw P, Liu W, Jumbe N, Miller M H, and Drusano G L. Pharmacodynamics of Daptomycin in a Murine Thigh Model of Staphylococcus aureus Infection. AAC 2001: 45(3), 845-851.
(3) Methods for dilution antimicrobial susceptibility tests for bacteria that grow aerobically; approved standard-10th Edition. Wayne (Pa.): Clinical and Laboratory Standards Institute (US); 2015 January 35(2). Report No: M07-A10.
(4) Schuch R, Lee H M, Schneider B C, Sauve K L, Law C, Khan B K, Rotolo J A, Horiuchi Y, Couto D E, Raz A, Fischetti V A, Huang D B, Nowinski R C, and Wittekind M. JID. Combination Therapy With Lysin CF-301 and Antibiotic Is Superior to Antibiotic Alone for Treating Methicillin-Resistant Staphylococcus aureus-Induced Murine Bacteremia. 2014; 209:1469-78.
(5) VanScoy B, Mendes R E, Nicasio A M, Castanheira M, Bulik C C, Okusanya O O, Bhavnani S M, Forrest A, Jones R N, Friedrich L V, Steenbergen J N, and Ambrose P G. Pharmacokinetics-Pharmacodynamics of Tazobactam in Combination with Ceftolozane in an In Vitro Infection Model. AAC 2013: 57, 2809-2814.
(6) Wadhwa, M. et al, “Immunogenicity assessment of biotherapeutic products: An overview of assays and their utility,” Biologicals, 2015, 43: 298-306; doi.org/10.1016/j.biologicals.2015.06.004.
(7) Soria-Guerra, S. E. et al, An overview of bioinformatics tools for epitope prediction: Implications on vaccine development, J. Biomed. Informatics 53 (2015) 405-414.
(8) Jawa V. et al, Clinical Immunology (2013) 149, 534-555.
(9) Entenza J M et al., “Therapeutic effects of bacteriophage Cpl-1 lysin against Streptococcus pneumoniae endocarditis in rats,” Antimicrob Agents Chemother. 2005 November; 49(11):4789-92.
(10) Yang, H. et al, Sci Rep. 2017 Jan. 9; 7:40182. doi: 10.1038/srep40182.
(11) Mazor, R. et al, “Dual B- and T-cell de-immunization of recombinant immunotoxin targeting mesothelin with high cytotoxic activity,” 2016, Oncotarget, 7(21): 29916.
(12) Blazanovic, K. et al, “Structure-based redesign of lysostaphin yields potent antistaphylococcal enzymes that evade immune surveillance,” 2015, Mol. Ther.—Meths & Clin. Dev. (2015) 2, 15021; doi:10.1038/mtm.2015.21.
(13) Zhao, H et al, “Depletion of T cell epitopes in lysostaphin mitigates anti-drug antibody response and enhances antibacterial efficacy in vivo,” Chem Biol., 2015, 22(5): 629-639. doi: 10.1016/j.chembiol.2015.04.017.
(14) De Groot, A et al, “Prediction of immunogenicity: in silico paradigms, ex vivo and in vivo correlates,” Curr. Opin. Pharmacol., 2008, 8:1-7.
(15) Parker A S, et al., “Structure-guided deimmunization of therapeutic proteins,” J. Comput. Biol. 2013, 20:152-165.
(16) King, C et al., “Removing T-cell epitopes with computational protein design,” 2014, PNAS 111(23): 8577-8582.
(17) Mazor, R. et al, PNAS|Jun. 10, 2014|vol. 111, no. 23: 8571-8576.
(18) Griswold, K. et al, Curr Opin Struct Biol. 2016 August; 39: 79-88; published online 2016 Jun. 17. doi: 10.1016/j.sbi.2016.06.003.

1. A modified lysin polypeptide comprising at least one amino acid substitution as compared to a wild-type PlySs2 lysin polypeptide having an amino acid sequence of SEQ ID NO: 1, a cysteine, histidine-dependent amidohydrolase/peptidase (CHAP) domain, and a cell wall binding (SH3b) domain, wherein the at least one amino acid substitution is in the CHAP domain and/or the SH3b domain, wherein the modified lysin polypeptide or fragment thereof inhibits the growth, reduces the population, or kills at least one species of Gram-positive bacteria.

2. The modified lysin polypeptide of claim 1, wherein the at least one amino acid substitution is in the CHAP domain in at least one position selected from amino acid residue 35, 92, 104, 128, and 137 of SEQ ID NO: 1 and/or in the SH3b domain in at least one position selected from amino acid residue 164, 184, 195, 198, 204, 206, 212, and 214 of SEQ ID NO: 1.

3. The modified lysin polypeptide of claim 2, wherein the at least one amino acid substitution in the CHAP domain is at least one of R35E, L92W, V104S, V128T and Y137S.

4. The modified lysin polypeptide of claim 2, wherein the at least one amino acid substitution in the SH3b domain is at least one of Y164N, Y164K, N184D, R195E, S198H, S198Q, V204K, V204A, I206E, V212A, V212E, and V214G.

5. The modified lysin polypeptide of claim 2, wherein the modified lysin polypeptide has at least one amino acid substitution in the CHAP domain selected from the group consisting of R35E, L92W, V104S, V128T and Y137S and at least one amino acid substitution in the SH3b domain selected from the group consisting of Y164N, Y164K, N184D, R195E, S198H, S198Q, V204K, V204A, I206E, V212A, V212E, and V214G.

6. The modified lysin polypeptide of claim 1, comprising at least two amino acid substitutions in the CHAP domain.

7. The modified lysin polypeptide of claim 1, comprising at least two amino acid substitutions or at least three amino acid substitutions in the SH3b domain.

8. The modified lysin polypeptide of claim 1, wherein the modified lysin polypeptide comprises 3-9 amino acid substitutions as compared to SEQ ID NO: 1, wherein the 3-9 amino acid substitutions are selected from: R35E, L92W, V104S, V128T, Y137S, Y164N, Y164K, N184D, R195E, S198H, S198Q, V204K, V204A, 1206E, V212E, V212A, and V214G.

9. The modified lysin polypeptide of claim 1, wherein the at least one amino acid substitution comprises L92W, V104S, V128T, Y137S, Y164K, N184D, and S198Q.

10. The modified lysin polypeptide of claim 1, wherein the at least one amino acid substitution in the CHAP domain comprises L92W, V104S, V128T and Y137S.

11. The modified lysin polypeptide of claim 1, wherein the at least one amino acid substitution is selected from the group consisting of:
(i) L92W, V104S, V128T, and Y137S;
(ii) Y164N, N184D, R195E, V204K, and V212E;
(iii) L92W, V104S, V128T, Y137S, S198H, and 1206E;
(iv) L92W, V104S, V128T, Y137S, S198Q, V204A, and V212A;
(v) L92W, V104S, V128T, Y137S, Y164K, N184D, and S198Q;
(vi) V128T, Y137S, and Y164K;
(vii) R35E, L92W, V104S, V128T, and Y137S;
(viii) L92W, V104S, V128T, Y137S, Y164K, V204K, and V212E;
(ix) L92W, V104S, V128T, Y137S, Y164K, N184D, S198Q, V204K, and V212E;
(x) L92W, V104S, V128T, Y137S, Y164N, and N184D;
(xi) L92W, V104S, V128T, Y137S, Y164N, and R195E;
(xii) L92W, V104S, V128T, Y137S, N184D, V204A, and V212A;
(xiii) L92W, V104S, V128T, Y137S, and Y164K;
(xiv) L92W, V104S, V128T, Y137S, Y164K, 1206E, and V214G; and
(xv) L92W, V104S, V128T, Y137S, N184D, and S198H.

12. The modified lysin polypeptide of claim 1, having a minimum inhibitory concentration (MIC) no greater than about 2, about 3, or about 5 times that of a wild-type PlySs2 lysin against one or more of Staphylococcus aureus; Listeria monocytogenes; Staphylococcus aureus; a coagulase negative staphylococcus such as from the Staphylococcus epidermidis group, the Staphylococcus saprophyticus group, the Staphylococcus simulans group, the Staphylococcus intermedius group, the Staphylococcus sciuri group, and the Staphylococcus hyicus group; Streptococcus suis; Streptococcus pyogenes; Streptococcus agalactiae; Streptococcus dysgalactiae; Streptococcus pneumoniae; species included in the viridans streptococci group such as the Streptococcus anginosis group, Streptococcus mitis group, Streptococcus sanguinis group, Streptococcus bovis group, Streptococcus salivarius group, and Streptococcus mutans group; Enterococcus faecalis; and Enterococcus faecium.

13. The modified lysin polypeptide of claim 12, wherein the MIC is no greater than about 5 times that of the wild-type PlySs2 lysin against one or more of Staphylococcus aureus, Streptococcus pyogenes, Listeria monocytogenes, and Streptococcus agalactiae.

14. The modified lysin polypeptide of claim 12, wherein the MIC is no greater than about 4 times that of the wild-type PlySs2 lysin.

15. The modified lysin polypeptide of claim 12, wherein the MIC is no greater than about 2 times that of the wild-type PlySs2 lysin.

16. The modified lysin polypeptide of claim 1, wherein the modified lysin polypeptide reduces immunogenicity and/or reduces inflammatory response-related toxicity compared to wild-type PlySs2 lysin.

17. The modified lysin polypeptide of claim 1, wherein inhibiting the growth, reducing the population, or killing at least one species of Gram-positive bacteria is assessed in vitro as a minimum inhibitory concentration (MIC) and/or a minimum biofilm eradication concentration (MBEC).

18. A composition comprising an acceptable carrier and an antibacterial amount of the modified lysin polypeptide according claim 1.

19. The composition of claim 18, wherein the composition is a pharmaceutical composition and the carrier is a pharmaceutically acceptable carrier.

20. The composition of claim 18, wherein the antibacterial amount of the modified lysin polypeptide is effective to inhibit the growth, or reduce the population, or kill one or more Gram-positive bacteria selected from the group consisting of Staphylococcus aureus; Listeria monocytogenes; a coagulase negative staphylococcus such as from the Staphylococcus epidermidis group, the Staphylococcus saprophyticus group, the Staphylococcus simulans group, the Staphylococcus intermedius group, the Staphylococcus sciuri group, and the Staphylococcus hyicus group; Streptococcus suis; Streptococcus pyogenes; Streptococcus agalactiae; Streptococcus dysgalactiae; Streptococcus pneumoniae; species included in the viridans streptococci group such as the Streptococcus anginosis group, Streptococcus mitis group, Streptococcus sanguinis group, Streptococcus bovis group, Streptococcus salivarius group, and Streptococcus mutans group; Enterococcus faecalis; and Enterococcus faecium.

21. The composition of claim 20, wherein the Gram-positive bacteria is a methicillin-resistant Staphylococcus aureus or a vancomycin-resistant Staphylococcus aureus.

22. The composition of claim 18, which is a solution, a suspension, an emulsion, an inhalable powder, an aerosol, or a spray.

23. The composition of claim 18, further comprising one or more antibiotics suitable for the treatment of a Gram-positive bacterial infection.

24. A nucleic acid molecule encoding the modified lysin polypeptide of claim 1.

25. A vector comprising the nucleic acid molecule of claim 24.

26. The vector of claim 25, wherein the vector is a plasmid and the nucleic acid is operatively linked to a heterologous promoter.

27. A method of inhibiting the growth, reducing the population, or killing of at least one species of Gram-positive bacteria, the method comprising contacting the bacteria with the composition of claim 18.

28. A method of preventing or treating a bacterial infection caused by at least one species of Gram-positive bacteria, the method comprising co-administering to a subject diagnosed with, at risk for, or exhibiting symptoms of a bacterial infection (1) a first amount of the modified lysin polypeptide of claim 1; and (2) a second amount of an antibiotic suitable for the treatment of a Gram-positive bacterial infection.

29. The method of claim 28 wherein the antibiotic suitable for the treatment of the Gram-positive bacterial infection is selected from the group consisting of methicillin, vancomycin, daptomycin, mupirocin, and lysostaphin.

30. A method for augmenting the efficacy of an antibiotic suitable for the treatment of a bacterial infection, the method comprising co-administering the antibiotic in combination with the modified lysin polypeptide of claim 1, wherein co-administration is more effective in inhibiting the growth, or reducing the population, or killing the bacteria than administration of either the antibiotic or the modified lysin polypeptide or fragment thereof individually.

31. The method of claim 30, wherein the antibiotic is selected from the group consisting of methicillin, vancomycin, daptomycin, mupirocin and lysostaphin.

32. A method of reducing the development of antibiotic resistance in Staphylococcus or Streptococcus bacteria in a subject infected with Staphylococcus or Streptococcus bacteria, the method comprising administering to the subject a combination of the modified lysin polypeptide of claim 1 and an antibiotic selected from methicillin, vancomycin, daptomycin, mupirocin, and lysostaphin.

33. The method of claim 32, wherein the modified lysin polypeptide is administered in an amount corresponding to a concentration below the minimal inhibitory concentration (MIC) of the modified lysin polypeptide.

34. The method of claim 32, wherein the at least one amino acid substitution in the modified lysin polypeptide comprises L92W, V104S, V128T, Y137S, S198Q, V204A, and V212A.
